SEQUENCE LISTING (39 sequences)

SEQ ID NO: 1 - protein, 22 aa, synthetic construct
YARVRRRGPRGYARVRRRGPRR

SEQ ID NO: 2 - RNA, 30 nt, synthetic construct
  source 1..30: mol_type = other RNA
gacaacagtttgccgctgcccaatgccatc

SEQ ID NO: 3 - RNA, 30 nt, synthetic construct
  source 1..30: mol_type = other RNA
agttgctgctcttttccaggttcaagtggg

SEQ ID NO: 4 - RNA, 30 nt, synthetic construct
  source 1..30: mol_type = other RNA
gtttgagaattccctggcgcaggggcaact

SEQ ID NO: 5 - RNA, 30 nt, synthetic construct
  source 1..30: mol_type = other RNA
caatttctccttgtttctcaggtaaagctc

SEQ ID NO: 6 - RNA, 30 nt, synthetic construct
  source 1..30: mol_type = other RNA
cagatgatttaactgctcttcaaggtcttc

SEQ ID NO: 7 - RNA, 30 nt, synthetic construct
  source 1..30: mol_type = other RNA
atctcttccacatccggttgtttagcttga

SEQ ID NO: 8 - RNA, 30 nt, synthetic construct
  source 1..30: mol_type = other RNA
gtaaacggtttaccgccttccactcagagc

SEQ ID NO: 9 - RNA, 30 nt, synthetic construct
  source 1..30: mol_type = other RNA
gtgtcaccagagtaacagtctgagtaggag

SEQ ID NO: 10 - RNA, 30 nt, synthetic construct
  source 1..30: mol_type = other RNA
ggtaatgagttcttccaactggggacgcct

SEQ ID NO: 11 - RNA, 30 nt, synthetic construct
  source 1..30: mol_type = other RNA
cctccggttctgaaggtgttcttgtacttc

SEQ ID NO: 12 - RNA, 30 nt, synthetic construct
  source 1..30: mol_type = other RNA
gagaagtttcagggccaagtcatttgccac

SEQ ID NO: 13 - RNA, 30 nt, synthetic construct
  source 1..30: mol_type = other RNA
tcttccaaagcagcctctcgctcactcacc

SEQ ID NO: 14 - RNA, 18 nt, synthetic construct
  source 1..18: mol_type = other RNA
tcactttcataatgctgg

SEQ ID NO: 15 - RNA, 19 nt, synthetic construct
  source 1..19: mol_type = other RNA
gacaagggcgatttgacag

SEQ ID NO: 16 - RNA, 20 nt, synthetic construct
  source 1..20: mol_type = other RNA
tccgaagttcactccacttg

SEQ ID NO: 17 - RNA, 22 nt, synthetic construct
  source 1..22: mol_type = other RNA
cagccagtgaagaggaagttag

SEQ ID NO: 18 - RNA, 20 nt, synthetic construct
  source 1..20: mol_type = other RNA
ccagccattgtgttgaatcc

SEQ ID NO: 19 - RNA, 20 nt, synthetic construct
  source 1..20: mol_type = other RNA
tccctgagctgaacgggaag

SEQ ID NO: 20 - RNA, 20 nt, synthetic construct
  source 1..20: mol_type = other RNA
ggaggagtgggtgtcgctgt

SEQ ID NO: 21 - RNA, 21 nt, synthetic construct
  source 1..21: mol_type = other RNA
caactttggcattgtggaagg

SEQ ID NO: 22 - RNA, 20 nt, synthetic construct
  source 1..20: mol_type = other RNA
gaagagtgggagttgctgtt

SEQ ID NO: 23 - RNA, 30 nt, synthetic construct
  source 1..30: mol_type = other RNA
aaaacgccgccatttctcaacagatctgtc

SEQ ID NO: 24 - RNA, 30 nt, synthetic construct
  source 1..30: mol_type = other RNA
gccgccatttctcaacagatctgtcaaatc

SEQ ID NO: 25 - RNA, 30 nt, synthetic construct
  source 1..30: mol_type = other RNA
cataatgaaaacgccgccatttctcaacag

SEQ ID NO: 26 - RNA, 30 nt, synthetic construct
  source 1..30: mol_type = other RNA
cgccatttctcaacagatctgtcaaatcgc

SEQ ID NO: 27 - RNA, 30 nt, synthetic construct
  source 1..30: mol_type = other RNA
atatcataatgaaaacgccgccatttctca

SEQ ID NO: 28 - RNA, 30 nt, synthetic construct
  source 1..30: mol_type = other RNA
actgttcagcttctgttagccactgattaa

SEQ ID NO: 29 - RNA, 30 nt, synthetic construct
  source 1..30: mol_type = other RNA
cttaagataccatttgtatttagcatgttc

SEQ ID NO: 30 - RNA, 30 nt, synthetic construct
  source 1..30: mol_type = other RNA
attctcaggaatttgtgtctttctgagaaa

SEQ ID NO: 31 - RNA, 21 nt, synthetic construct
  source 1..21: mol_type = other RNA
ataacaccaccactcttactc

SEQ ID NO: 32 - RNA, 21 nt, synthetic construct
  source 1..21: mol_type = other RNA
gtagccgtgatgccattgtca

SEQ ID NO: 33 - RNA, 21 nt, synthetic construct
  source 1..21: mol_type = other RNA
agcctgaagaacgagatcagc

SEQ ID NO: 34 - RNA, 29 nt, synthetic construct
  source 1..29: mol_type = other RNA
gctatcatactggctattatatgggtttt

SEQ ID NO: 35 - RNA, 25 nt, synthetic construct
  source 1..25: mol_type = other RNA
ctctatgccagcatttctccttaat

SEQ ID NO: 36 - RNA, 22 nt, synthetic construct
  source 1..22: mol_type = other RNA
tctggaccaccaataattcccc

SEQ ID NO: 37 - RNA, 25 nt, synthetic construct
  source 1..25: mol_type = other RNA
atgccagcatttccatataatagcc

SEQ ID NO: 38 - RNA, 17 nt, synthetic construct
  source 1..17: mol_type = other RNA
gagaaacctgccaagta

SEQ ID NO: 39 - RNA, 17 nt, synthetic construct
  source 1..17: mol_type = other RNA
cagtgtagcccaagatg

(19) United States
(12) Patent Application Publication
(10) Pub. No.: US 2025/0114464 A1
(43) Pub. Date: Apr. 10, 2025

(54) PRE-MRNA SLICE MODULATING PEPTIDE-CONJUGATED ANTISENSE THERAPEUTICS FOR THE TREATMENT OF DISEASES

(71) Applicants: The Governors of the University of Alberta, Edmonton (CA); Oregon State University, Corvalis, OR (US)

(72) Inventors: Toshifumi YOKOTA, Edmonton (CA); Rika YOKOTA-MARUYAMA, Edmonton (CA); Hong M. MOULTON, Corvalis, OR (US)

(21) Appl. No.: 18/730,955

(22) PCT Filed: Jan. 26, 2023
(86) PCT No.: PCT/CA2023/050096

§ 371 Date: Jul. 22, 2024

Related U.S. Application Data
(60) Provisional application No. 63/303,577, filed on Jan. 27, 2022.

(51) Int. Cl. A61K 47/64; A61P 21/00; C12N 15/113

(52) U.S. Cl. CPC A61K 47/64; A61P 21/00; C12N 15/113; C12N 2310/11; C12N 2310/3233; C12N 2310/3513; C12N 2320/33

(57) ABSTRACT

The present disclosure relates generally to DG9 peptide-conjugated splice modulating therapies for the treatment of Duchenne muscular dystrophy and spinal muscular atrophy.

This application claims priority to U.S. Provisional Application Ser. No. 63/303,577 filed Jan. 27, 2022 which is hereby incorporated by reference in its entirety.

### FIELD OF THE INVENTION

The present invention relates generally to pre-mRNA splice modulating peptide-conjugated antisense therapeutics for treatment of diseases and in particular, peptide-conjugated antisense therapeutics for the treatment of Duchenne Muscular Dystrophy (DMD) and Spinal Muscular Atrophy (SMA).

### BACKGROUND

Antisense therapeutics that modulate pre-messenger RNA splicing by either inducing exon splicing or inhibiting exon splicing function by binding to the pre-messenger RNA and have been approved for the treatment of various diseases including Duchenne's Muscular Dystrophy and Spinal Muscular Atrophy.

Duchenne Muscular Dystrophy (DMD) is a fatal, X-linked recessive disorder caused by mutations in the DMD gene that lead to absence of dystrophin in muscle. Dystrophin stabilizes the sarcolemma by bridging cytoskeletal actin to the extracellular matrix, via forming a membrane-associated glycoprotein complex [1, 2]. Dystrophin loss results in progressive body-wide muscle degeneration, loss of ambulation before the teens, and cardiorespiratory malfunction during the twenties that typically leads to death [3]. DMD is present in 19.8 per 100,000 male births and is the most common inherited neuromuscular disorder worldwide [4].

Most patients (˜70%) have large out-of-frame deletions in DMD [5]. Exon skipping based therapeutics are based on the observation that, at least ˜90% of the time, in-frame mutations in DMD give rise to milder phenotypes, as found in Becker Muscular Dystrophy (BMD) [5, 6]. By excluding out-of-frame exons from the DMD transcript using antisense oligonucleotides, exon skipping converts out-of-frame into in-frame mutations, producing truncated but partially functional dystrophin. Four exon skipping therapies have been approved by the U.S. FDA: eteplirsen/Exondys 51 (Sarepta) [7], golodirsen/Vyondys 53 (Sarepta) [8], viltolarsen/Viltepso (NS Pharma) [9], and casimersen/Amondys45 (Sarepta) [10]. All are phosphorodiamidate morpholino oligomer (PMO) antisense oligonucleotides.

The applicability of single-exon skipping is, however, limited due to its mutation-specific nature of DMD. The therapies above could each treat at most only 8-13% of all patients [11]. Multi-exon skipping overcomes this issue, particularly skipping exons 45-55. Exons 45-55 is a mutation hotspot in the DMD gene, harboring 66% of large (≥1 exon) deletions and 15% of large duplications in patients (5). Exons 45-55 skipping could theoretically treat more than 40% of all DMD patients [12].

Deletion of exons 45-55 is also commonly associated with asymptomatic to mild phenotypes. In the Leiden DMD database, 90% of patients with the exons 45-55 deletion have BMD; in other databases, including UMD-TREAT-NMD, and clinical studies, the deletion is associated with BMD or asymptomatic individuals in all examined cases [12-15].

Exons 45-55 skipping cocktails that restored dystrophin synthesis in the muscles of dystrophic mice have been developed [16]. A PMO cocktail that skipped human DMD exons 45-55 in immortalized patient myotubes and humanized DMD mice have been developed [13, 17]. Average skipping efficacies of 27-61% and 15-22% were observed, respectively, and treatment produced up to 14% dystrophin of normal levels in vitro [13, 17]. However, this cocktail used one PMO per exon (except exon 48, which required two) to skip exons 45-55. Efficacy is reduced and off-target effects increased as all PMOs have to be present in the same nuclei to induce skipping of exons 45-55, PMO based exon skipping therapies efficacy is impacted by their rapid clearance from the bloodstream, poor uptake into muscle, and inability to escape from endosomes [19, 20]. Eteplirsen for instance, only restored 0.93% dystrophin of normal levels in patients after 180 weeks of treatment with a 30 or 50 mg/kg/week dose [7].

Accordingly, there exists a need for improved exon-skipping therapies for the treatment of Duchene Muscular Dystrophy.

Spinal muscular atrophy (SMA) is a devastating neurodegenerative disorder affecting motor neurons in the anterior horn of the spinal cord [45]. SMA can result in progressive muscular weakness, respiratory distress, or even paralysis, and is one of the leading genetic causes of infant mortality [46]. SMA is characterized by mutations in the survival of the telomeric motor neuron 1 (SMN1) gene leading to its homozygous deletion [47]. The complete loss of SMN protein is embryonically lethal [48,49]. Humans possess a paralog of SMN1 called SMN2, enabling patients to be born [47,50]. However, SMN2 cannot fully compensate for SMN1 loss because of a C-to-T transition in exon 7, leading to its exclusion.

While the full-length SMN2 (FL-SMN2) transcripts can produce a stable functional protein (˜10%), the transcripts without exon 7 (A7 SMN2) are unstable, and the protein produced is rapidly degraded (˜90%) [51]. Exclusion of SMN2 exon 7 is due to ISS-N1 (intronic splicing silencer N1) that binds to a repressor protein hnRNP A1 (heterogeneous ribonucleoprotein A1) [52-54].

All SMA patients lack a functional SMN1 gene and therefore, are dependent on SMN2 gene to produce SMN protein necessary for survival. The number of copies of SMN2 is therefore a potent genetic modifier of SMA, with the copy number correlating inversely with the severity of SMA [55,56]. The inability of SMN2 to compensate for SMN1 loss is responsible for the preferential degeneration of motor neurons [51,57]. Although the pathological hallmark of SMA is motor neuron degeneration, recent studies highlight defects in peripheral tissues [58-60]. Additionally, in vivo mouse studies have reported bradycardia and dilated cardiomyopathy, which improved with restoration of SMN levels [61-63]. While the reason remains unclear, motor neurons are particularly susceptible to the loss of SMN [64].

Currently, three SMA-modifying treatments have been approved by the U.S. Food and Drug Administration (FDA): nusinersen (brand name Spinraza), onasemnogene abeparvovec (brand name Zolgensma), and risdiplam (brand name Evrysdi). However, these three drugs have several associated concerns. Onasemnogene abeparvovec is gene-therapy drug and is only approved for patients less than 2 years of age [65]. Risdiplam is an orally deliverable small molecule drug. However, there are safety concerns due to its associated off-target effects on pre-mRNA splicing [66].

Nusinersen is a splice-switching antisense oligonucleotide (AO) with a 2′-O-methoxyethyl (MOE) phosphorothioate chemistry. It modulates SMN2 splicing by targeting ISS-N1 and promotes the inclusion of exon 7. Since nusinersen does not cross the blood-brain barrier (BBB), it requires repeated intrathecal administration that ensures a robust restoration of SMN protein mainly to the central nervous system (CNS) tissues [67]. Despite providing a safe profile, nearly one-third of the treated patients suffer from scoliosis, persistent lumbar-fluid leakage, thrombocytopenia, and other coagulation defects [68,69].

To overcome issues associated with intrathecal injections, several AO chemistries and modifications have been studied such as phosphorodiamidate morpholino oligomers (PMOs), locked nucleic acid (LNAs), and constrained ethyl (c-Et) [70]. The uncharged backbone, low protein binding affinity, and nuclease stability make PMO chemistry an ideal candidate as several studies have shown that the administration of multiple high doses can be achieved with minimal toxicity 70,71]. However, the main pitfall associated with PMO is low efficacy which is tied to its rapid clearance from the bloodstream, poor uptake in tissues such as the skeletal muscle, and endosome-trapping [72,73]. To improve delivery of PMOs to the nuclei in target tissues and reduce the administered dose, numerous cell-penetrating peptides (CPPs) conjugated PMOs are being studied. Some of these peptides facilitate the transport of PMOs across the BBB and thus ensure the possibility of non-invasive administration for the treatment of neuronal disorders [74]. Hammond et al. showed that the systemic administration of Pip6a, a peptide from the family of PMO internalizing peptide (Pip) into adult mice, increased the FL-SMN2 expression in the peripheral and CNS tissues [71]. However, the localization and distribution of AOs in the CNS remain unknown. Additionally, Pip peptides are known to be highly toxic [74].

Accordingly, there exists a need for improved splice modulating therapies for the treatment of Spinal Muscular Atrophy.

### SUMMARY OF THE INVENTION

The present invention provides pre-mRNA splice modulating peptide-conjugated antisense therapeutics for treatment of diseases.

In one aspect of the invention, there is provided a conjugate comprising an antisense oligonucleotide capable of modulating exon splicing of pre-mRNA attached to a cell penetrating peptide (CPP) comprising the amino acid sequence:

YArVRRrGPRGYArVRRrGPRr;

uppercase: L-amino acids, lowercase: D-amino acids.

In another aspect of the invention, there is provided a conjugate comprising an antisense oligonucleotide capable of inducing exon skipping in human dystrophin covalently attached to a cell penetrating peptide (CPP) comprising the amino acid sequence:

YArVRRrGPRGYArVRRrGPRr;

uppercase: L-amino acids, lowercase: D-amino acids.

In some embodiments, the conjugate capable of inducing exon skipping in human dystrophin has an antisense oligonucleotide that binds to a target in exon 44, exon 45, exon 46, exon 47, exon 48, exon 49, exon 50, exon 51, exon 52, exon 53, exon 54 and/or exon 55 of human dystrophin pre-mRNA.

In some embodiments, the conjugate capable of inducing exon skipping in human dystrophin has an antisense oligonucleotide that comprises or consists of any one of the following oligonucleotides:

5′-AAAACGCCGCCATTTCTCAACAGATCTGTC-3′,
5′-GACAACAGTTTGCCGOTGCCCAATGCCATC-3′,
5′-AGTTGCTGCTCTTTTCCAGGTTCAAGTGGG-3′,
5′-GTTTGAGAATTCCCTGGCGCAGGGGCAACT-3′,
5′-CAATTTCTCCTTGTTTCTCAGGTAAAGCTC-3′,
5′-CAGATGATTTAACTGCTCTTCAAGGTCTTC-3′,
5′-ATCTCTTCCACATCCGGTTGTTTAGCTTGA-3′,
5′-GTAAACGGTTTACCGCCTTCCACTCAGAGC-3′,
5′-GTGTCACCAGAGTAACAGTCTGAGTAGGAG-3′,
5′-GGTAATGAGTTCTTCCAACTGGGGACGCCT-3′,
5′-CCTCCGGTTCTGAAGGTGTTCTTGTACTTC-3′,
5′-GAGAAGTTTCAGGGCCAAGTCATTTGCCAC-3′,
and
5′-TCTTCCAAAGCAGCCTCTCGCTCACTCACC-3′.

In accordance with another aspect of the invention, there is provided a composition comprising a first conjugate comprising a first antisense oligonucleotide capable of inducing exon skipping in human dystrophin covalently attached to a cell penetrating peptide (CPP) comprising the amino acid sequence:

YArVRRrGPRGYArVRRrGPRr;

uppercase: L-amino acids, lowercase: D-amino acids and a second conjugate comprising a second antisense oligonucleotide capable of inducing exon skipping in human dystrophin covalently attached to the cell penetrating peptide (CPP).

In some embodiments, the composition, further comprises at least one other conjugate comprising another antisense oligonucleotide capable of inducing exon skipping in human dystrophin covalently attached to the cell penetrating peptide (CPP).

In some embodiments, the composition capable of inducing multi-exon skipping in human dystrophin comprises a peptide-conjugated antisense oligonucleotide targeting exon 45, a peptide-conjugated antisense oligonucleotide targeting exon 46, a peptide-conjugated antisense oligonucleotide targeting exon 47, a peptide-conjugated antisense oligonucleotide targeting exon 48, a peptide-conjugated antisense oligonucleotide targeting exon 49, a peptide-conjugated antisense oligonucleotide targeting exon 50, a peptide-conjugated antisense oligonucleotide targeting exon 51, a peptide-conjugated antisense oligonucleotide targeting exon 52, a peptide-conjugated antisense oligonucleotide targeting exon 53, a peptide-conjugated antisense oligonucleotide targeting exon 54 and a peptide-conjugated antisense oligonucleotide targeting exon 55.

In some embodiments, the composition capable of inducing multi-exon skipping in human dystrophin comprises a peptide-conjugated antisense oligonucleotide targeting exon 45, a peptide-conjugated antisense oligonucleotide targeting exon 47, a peptide-conjugated antisense oligonucleotide targeting exon 49, a peptide-conjugated antisense oligonucleotide targeting exon 51, a peptide-conjugated antisense oligonucleotide targeting exon 53 and a peptide-conjugated antisense oligonucleotide targeting exon 55.

In some embodiments, the composition capable of inducing multi-exon skipping in human dystrophin comprises a peptide-conjugated antisense oligonucleotide targeting exon 45, a peptide-conjugated antisense oligonucleotide targeting exon 47 and a peptide-conjugated antisense oligonucleotide targeting exon 53.

In accordance with an aspect of the invention, there is provided a method of treating a subject having DMD, comprising administering a therapeutically effective amount one or more peptide conjugates of the invention capable of inducing exon skipping in human dystrophin.

In accordance with another aspect of the invention, there is provided a conjugate comprising an antisense oligonucleotide capable of inducing exon inclusion in human SMN2 gene covalently attached to a cell penetrating peptide (CPP) comprising the amino acid sequence:

YArVRRrGPRGYArVRRrGPRr;

uppercase: L-amino acids, lowercase: D-amino acids.

In some embodiments, the conjugate comprising an antisense oligonucleotide capable of inducing exon inclusion in human SMN2 gene binds to intronic splicing silencer N1 of SMN2 pre-nIRNA.

In some embodiments, the conjugate comprising an antisense oligonucleotide capable of inducing exon inclusion in human SMN2 gene includes the antisense oligonucleotide comprises or consists of the sequence 5′-TCACTTTCATAATGCTGG-3′ and wherein the thymines are optionally replaced with uracil, optionally wherein the antisense oligonucleotide is a phosphorodiamidate morpholino oligomer.

In accordance with another aspect of the invention, there is provided a method of treating spinal muscular atrophy (SMA) in a subject, comprising administering a therapeutically effective amount of the conjugate comprising an antisense oligonucleotide capable of inducing exon inclusion in human SMN2 gene of the invention.

In accordance with another aspect of the invention, there is provided a pharmaceutical composition comprising one or more peptide conjugates of the invention, and a pharmaceutically acceptable excipient.

### BRIEF DESCRIPTION OF THE FIGURES

Embodiments of the present invention will now be described, by way of example only, with reference to the attached Figures.

FIG. 1 shows testing minimized exons 45-55 skipping cocktails in immortalized patient myotubes. (A) The DMD exons targeted by the “all” cocktail and its derivatives are indicated by circles. (B) Culture scheme for PMO transfection in immortalized patient myotubes. (C) RT-PCR exons 45-55 skipping efficiency results upon transfection of PMO cocktails in KM155, KM571, 6594, and 6311 myotubes. Black (upper) arrows indicate native/unskipped bands, blue (lower) arrows indicate exons 45-55-skipped bands. GAPDH is shown below as a control. Quantification is shown at the bottom. (D) Western blot for dystrophin in PMO-treated and non-treated (NT) KM571 myotubes using various antibodies (ab15277, DYS1, MANEX45A, MANEX4850E). Protein extracts were loaded at 40 μg for treated and NT samples, and at indicated percentages of this for wild-type KM155 samples (WT). Desmin was detected as a loading control. (E) Quantification of DYS1 signals in (D), relative to the intensity of the 12.5% WT band. (n=3 for C-E) Error: S.E.M. *p<0.05, **p<0.005, ***p<0.001 one-way ANOVA with Dunnett's test versus mock, ^{φ}p<0.05, ^{φφφ}p<0.001 one-way ANOVA with Dunnett's test versus “all”.

FIG. 2 shows single-dose exon 51 skipping treatment with DG9-PMO. (A) Male, 3-month-old hDMDdel52;mdx mice were given a single retro-orbital injection (1× r.o.) of saline, 50 mg/kg PMO, or equimolar 64 mg/kg DG9-PMO for exon 51 skipping. Tissues were collected 1 week later. (B) RT-PCR exon 51 skipping efficiency results post-treatment in various muscles, with quantification on the right. Gapdh is shown as a control. (C) Western blot for dystrophin (DYS1), with wild-type (WT) shown for reference. Protein extracts were loaded at 40 μg for saline-injected and treated muscles, and at indicated percentages of this for WT tibialis anterior samples. Desmin and myosin heavy chain (MyHC) serve as loading controls. Quantification of dystrophin signals are shown relative to the intensity of the 5% WT band. (D) Representative immunofluorescence images for dystrophin (DYS1, green) and nuclei (DAPI, blue) in various muscles and treatment conditions. Scale bar: 100 μm. (n=3/group for B-D) Error: S.E.M. *p<0.05, **p<0.005, ***p<0.001 one-way ANOVA with Tukey's test. TA/T, tibialis anterior; GAS/G, gastrocnemius; QUA/Q, quadriceps; DIA/D, diaphragm: HRT/H, heart.

FIG. 3 shows repeated-dose exon 51 skipping treatment with DG9-PMO. (A) Male, 2-month-old hDMDdel52;mdx mice were given three once-weekly retro-orbital injections (3× r.o.) of saline or 30 mg/kg DG9-PMO for exon 51 skipping. Purple arrows indicate functional testing. Tissues were collected 2 weeks later. (B) RT-PCR exon 51 skipping efficiency results post-treatment in various muscles, with quantification on the right. Gapdh is shown as a control. (C) Western blot for dystrophin (DYS1) in various skeletal muscles or (D) the heart, with corresponding wild-type (WT) samples used for reference. Protein extracts were loaded at 40 μg for saline-injected and treated muscles, and at indicated percentages of this for WAT. Desmin and myosin heavy chain (MyHC) serve as loading controls. Quantification of dystrophin signals are shown relative to the intensity of the 5% WT band. (n=3/group for B-D) Error: S.E.M. *p<0.05, ***p<0.001 unpaired two-tailed t-test for (B) to (D). (E) Representative immunofluorescence images for dystrophin (DYS1, green) and nuclei (DAPI, blue) in various muscles and conditions. Scale bar: 100 μm. (n=3/group) (F) Body weights of saline- and DG9-PMO-treated mice throughout the experiment. (G) Forelimb grip strength for saline- and DG9-PMO-treated mice, normalized to body weight. The % change from baseline is on the right. (H) Similar to (G), but for total limb grip strength. (n=11-14, WT: n=4, saline; n=6, DG9-PMO for F-H) Error: S.E.M. *p<0.05, **p<0.005 one-way ANOVA with Tukey's test for (G) to (H). TA/T, tibialis anterior; QUA/Q, quadriceps; DIA/D, diaphragm; HRT/H, heart.

FIG. 4 shows local treatment with the minimized “block” DG9-PMO exons 45-55 skipping cocktail. (A) Male, 5-6-month-old hDMDdel52;mdx mice were injected with the DG9-conjugated “block” cocktail at 5 μg/DG9-PMO (25 μg total dose) in the right tibialis anterior (R), and saline in the left (L). Tissues were collected 1 week later. (B) RT-PCR exons 45-55 skipping efficiency results post-treatment, with quantification on the right. Gapdh is shown as a control. (C) Western blot for dystrophin (DYS1), with wild-type (WT) and non-treated (NT) tibialis anterior samples used for reference. Protein extracts were loaded at 40 μg for NT, saline- and DG9-PMO-treated muscles, and at indicated percentages of this for WT. Desmin and myosin heavy chain (MyHC) serve as loading controls. Quantification of dystrophin signals are shown relative to the intensity of the 1% WAT band. (D) Representative immunofluorescence images for dystrophin (DYS1, green) and nuclei (DAPI, blue). Scale bar: 100 μm. (n=3, wild-type; n=6, saline; n=6, DG9-PMO) Error: S.E.M. *p<0.05, ***p<0.001 unpaired two-tailed t-test.

FIG. 5 details preliminary testing of minimized exons 45-55 skipping cocktails. Minimized derivatives of the “all” exons 45-55 skipping PMO cocktail were generated and tested in immortalized myotubes. (A) Strategy #1 for minimization involved two rounds of sequential removal of individual PMOs from the “all” cocktail. (B) Strategy #2 involved preparing “all” cocktail derivatives based on the endogenous splicing of the exons 45-55 region in humans. RT-PCR exons 45-55 skipping efficiency results are shown in both (A) and (B). (n=3) Error bars: S.E.M. *p<0.05,**p<0.005, ***p<0.001 one-way ANOVA with Dunnett's test versus mock, ^{φ}p<0.05, ^{φφ}p<0.005, ^{φφφ}p<0.001 one-way ANOVA with Dunnett's test versus “all”. NT, non-treated.

FIG. 6 shows histological data from single-dose exon 51 skipping treatment with DG9-PMO. Male, 3-month-old hDMDdel52;mdx mice were injected once retro-orbitally with saline, 50 mg/kg PMO, or equimolar 64 mg/kg DG9-PMO for exon 51 skipping. Tissues were collected 1 week later for assessment, sectioned, and stained using hematoxylin and eosin (HE). (A) Representative HE images of the tibialis anterior and diaphragm from wild-type, saline-, PMO-, and DG9-PMO-treated mice. Scale bar: 100 μm (B) Centrally nucleated fiber (CNF) quantification from HE images. Error bars: S.E.M. (C) Minimal Feret's diameter quantification from HE images for the tibialis anterior and (D) diaphragm. The frequency distribution is shown on the left, while quantification of individual fibers are shown on the right. Box edg^{es}, 25th ^{an}d 75th percentiles; central line, median; whiskers, range. (n=3/group for A-D, 719-854 fibers counted for the tibialis anterior and 962-1,479 for the diaphragm per group) **p<0.005, ***p<0.001 one-way ANOVA with Tukey's test.

FIG. 7 shows functional and histological data from repeated-dose exon 51 skipping treatment with DG9-PMO. Male, 2-month-old hDMDdel52;mdx mice were injected thrice retro-orbitally with saline or 30 mg/kg DG9-PMO for exon 51 skipping, once a week for 3 weeks. Functional testing was done at baseline and at 2 weeks after the final injection. (A) Rotarod test results showing average and peak fall times, as well as their respective % change values from baseline. (B) Run-to-exhaustion test results showing total distance travelled on the treadmill, with the % change from baseline on the right. (n=8-11 wild-type; n=2, saline; n=6, DG9-PMO) Error bars: S.E.M.*p<0.05, unpaired two-tailed t-test. (C) Tissues were collected after post-treatment functional testing and stained using hematoxylin and eosin (HE). Representative HE images of the tibialis anterior and diaphragm from wild-type, saline-, and DG9-PMO-treated mice are shown. Scale bar: 100 μm (D) Centrally nucleated fiber (CNF) quantification from HE images. Error bars: SIM. (E) Minimal Feret's diameter quantification for the tibialis anterior and (F) diaphragm, presented as in FIG. 6. (n=3/group for C-F, 610-865 fibers counted for the tibialis anterior and 1,167-1,412 for the diaphragm per group) **p<0.005, ***p<0.001 one-way ANOVA with Tukey's test.

FIG. 8 shows liver and kidney histology from single- and repeated-dose exon 51 skipping treatment studies. Representative hematoxylin and eosin-stained images of wild-type and hDMDdel52;mdx liver and kidney from the (A) single-dose and (B) repeated-dose experiments are shown. Scale bar: 100 μm. (n=3/group)

FIG. 9 shows subcutaneous administration of DG9-PMO at postnatal day 0 extends survival and improves motor function in severe SMA mice. (A) Survival curves of heterozygous mice (Het), non-treated (NT), unconjugated-PMO (PMO), DG09-PMO and MOE injected at PD0 at a dose of either 40 or 80 mg/kg. For 40 mg/kg studies, n=15 (Hets), n=22 (NT), n=49 (unconjugated PMO), n=14 (DG9-PMO), n=29 (MOE). For 80 mg/kg studies, n=15 (Hets), n=22 (NT), n=7 (unconjugated PMO), n=6 (DG9-PMO), n=4 (MOE) (p≤0.0001, log-rank Mantel Cox test). (B) Weight of mice at PD7 administered with either 40 or 80 mg/kg doses. Each dot (symbol) indicates a neonatal pup. (C) Hindlimb suspension assay (HLS). Mice were treated with 40 mg/kg AOs at PD0. Scored is based on the position of the hindlimbs when suspended from a falcon tube. (D) Righting reflex test. Mice were treated with 40 mg/kg AOs at PD0. The ability of mice to right themselves on their paws was measured every alternate day between PD2 to PD20 (left). The mean righting reflex time at PD6 and PD8 was also indicated (right: box whiskers plots). Box edges, 25^{th} and 75^{th} percentiles; central line, median; whiskerslange. (E) Forelimb grip strength measured in adult mice at PD30 and PD60 from 40 mg/kg treatment groups normalized to the body weight. In B,D (box whisker plots), and E, One-way ANOVA followed by post hoc Tukey's test was performed. *p<0.05, **p<0.01, ***p<0.005. In C and D, Two-way ANOVA followed by Sidak's multiple comparison in A and B. *p<0.03; **p<0.002; ***p<0.0002.*NT, #PMO, @DG9-PMO, &MOE. Error bars: SEM

FIG. 10 shows subcutaneous administration of DG9-PMO at postnatal day 0 increases SMN expression. (A) Relative expression levels of full length SMN2 (FL-SMN2) compared to deleted SMN2 transcripts (Δ7 SMN2) measured by qPCR. (B) Representative images from western blotting and the quantification SMN protein levels, relative to β-Tubulin. The heterozygous mice were used as a control with the relative SMN expression set to 1. 40 mg/kg AOs were injected on PD 0. Tissues were collected at PD7. One-way ANOVA followed by post hoc Tukey's test was performed *NT, #PMO, @DG9-PMO, &MOE. *p<0.05, **p<0.01, ***p<0.005. Error bars: SEM.

FIG. 11 shows DG9-PMO treatment improves breathing function at postnatal day 7 in SMA mice. (A) Representative traces of whole-body plethysmograph recording from P7 pups in normoxia (left column) and hypoxia (11% 02, right Column) (n=6 each). (B) Respiratory frequency f_{R}. (C) Tidal volume (V_{T}) relative to the mean of heterozygotes (100%) in normoxia. (0) Minute ventilation (V_{E}) relative to the mean of heterozygotes (100%) in normoxia. (E) Coefficient of variation of frequency (CV). (F) Total apnea duration (seconds in one minute). For those data (f_{R}, V_{T}, V_{E}, and CV in (B-E) that passed the normality test (Shapiro-Wilk) and equal variance test (Brown-Forsythe), parametric statistics were used with two-way repeated ANOVA, followed by Holm-Sidak method. The data did not pass the normality test, nonparametric statistics were used. (G) Correlation between respiratory frequency and body weight. Respiratory frequency was plotted against the body weight in heterozygotes (n=10) with a correlation coefficient of 0.303 (p=0.394). The homozygotes (n=43) had a correlation coefficient of 0.791 (p<0.001). 40 mg/kg AOs were injected on PD 0. Comparison of the difference in normoxia, or hypoxia was conducted with kruskal-Wallis one-way ANOVA on ranks, followed by Dunn's method. The difference between hypoxia and normoxia was conducted with a signed rank test. p<0.05 is taken as statistically significant difference; p<0.05, p<0.01, p<0.001 compared between groups indicated (*NT, #PMO, @DG9-PMO, &MOE); $<0.05, $$<0.01, $$$<0.001 compared with normoxia.

FIG. 12 shows systemic administration of DG9-PMO improves the muscle pathology in SMA mice. (A) Representative images from H&E staining of the quadriceps muscle (top row), diaphragm (middle row) and the intercostal muscle (bottom row) at PD7 in the heterozygous, NT control and treated groups. Scale bar: 100 μm. (B) Frequency distribution (Top) and the quantification (Bottom) of the minimal Feret's diameter (μm) of individual myofibers. Box edges, 25^{th} and 75^{th} percentiles; central line, median; whiskers, range (n=3-7 per group). 1292-1653 fibers for the quadriceps, 917-1746 fibers for the diaphragm and 642-1127 for the intercostal muscle were measured. (C) Centrally nucleated fibers quantified from the H & E images (%). 40 mg/kg AOs were injected on PD 0. Statistics was performed using one-way ANOVA followed by post hoc Tukey's test. *NT, #PMO, @DG9-PMO, &MOE. *p<0.05, **p<0.01, ***p<0.005. Error bars: SEM.

FIG. 13 shows DG9-PMO treatment leads to improvement in the neuromuscular junctions (NMJs).(A) Representative confocal images of the neuromuscular junction (NMJ) staining in quadriceps and intercostal muscles collected at PD30. Scale bar: 100 μm. Postsynaptic endplates were stained using a-bungarotoxin (red, a-BTX) while neurofilament (2H3) and synaptic vesicles (SV2) were indicative of neurons (green). Denervated endplates can be identified as a-BTX endplates without overlapping synaptophysin-stained axons, while partially denervated endplates are identified as <50% occupancy of the pre-synaptic nerve terminals in an endplate. White arrowheads: full innervation. Yellow arrows: partial innervation. Blue arrows: denervation. White arrows: collapsed NMJs. (B) Innervation characteristics: full innervation, partial innervation, denervation, collapsed NMJs were quantified from at least 300-500 NMJs per group (n=3-7) and plotted as percentages of total NMJs analyzed. (C) The number of collapsed vesicles (flat, non-pretzel shaped) were quantified (n=4-6 per group). In (B) two-way ANOVA followed by Sidak's multiple comparison was used. *p<0.03; **p<0.002; ***p<0.0002. In c. statistics was performed using one-way ANOVA followed by post hoc Tukey's test. #PMO, @DG9-PMO, &MOEL *p<0.05, **p<0,01, ***p<0.005. Error bars: sEM, 40 mg/kg AOs were injected on PD 0.

FIG. 14 shows DG9 increases uptake of PMO in target tissues following subcutaneous administration at PD0. (A) Concentrations of PMO (pM) were detected by ELISA using the avidin-biotin affinity system and compared between DG9-PMO and unconjugated-PMO treatments at 40 mg/kg doses in the quadriceps, liver, heart, kidney, brain, and the spinal cord (n=3-7 per group).*p<0.05, **p<0.01, ***p<0.001 unpaired two-tailed student's t-test. Error bars: SEM. (B) Representative immunohistochemistry images from PD7 heart, quadriceps muscle, brain, and the spinal cord following fluorescently tagged DG9-PMO subcutaneous administration at PD0. Green: Fluorescein-DG9-PMO. Magenta: DAPI. DG9-PMO without fluorescent tag was used for the negative control. White arrows indicate DG9-PMO overlapped with Nuclei (DAPI). n=3. Scale bar:50 μm.

FIG. 15 shows DG9-PMO penetrates the blood-brain barrier and increases FL-SMN2 expression in a mild SMA model. (A) Representative immunohistochemistry images at PD7 from the quadriceps muscle, heart, brain, and the spinal cord, following fluorescently tagged DG9-PMO (green) subcutaneous administration at PD5 in F0 mice (Smn^{-/-} SMN2^{+/+}). Magenta: DAPI. n=3 per group. White arrows indicate DG9-PMO overlapped with nuclei (DAPI). Scale bar: 50 μm. (B) Concentrations of PMO (pM) at PD7 were detected by ELISA using the avidin-biotin affinity system and compared between DG9-PMO and unconjugated-PMO treatments at 40 mg/kg injected at PD5 in the milder SMA model (n=3-6 per group). Statistics performed using unpaired two-tailed student's t-test. *p<0.05, **p<0.01, ***p<0.001 (C) Relative expression levels of full length SMN2 (FL-SMN2) compared to deleted SMN2 transcripts (Δ7 SMN2 in the quadriceps muscle, liver, heart, spleen, brain, and spinal cord. Saline, unconjugated-PMO, and DG9-PMO were injected into PD5 mice subcutaneously (n=3-6 per group). The tissues were collected at PD7. In c, one-way ANOVA followed by post hoc Tukey's test was performed. *p<0.05, **p<0.01, ***p<0.005. Error bars: SEM.

FIG. 16 shows DG9-PMO treatment induces SMN expression in a dose-dependent manner in SMA mice. (A) Relative expression levels of full length SMN2 (FL-SMN2) compared to deleted SMN2 transcripts (Δ7 SMN2) measured by qPCR in NT and treated mice in the quadriceps muscle, liver, heart, brain, and spinal cord following 80 mg/kg AO treatment. (B) Representative images from western blotting and the quantification of SMN levels relative to β-Tubulin. The heterozygous mice were used as a control relative SMN expression set to 1. The tissues from the 80 mg/kg treated mice were collected at PD7. In A-B, one-way ANOVA followed by post hoc Tukey's test was performed *NT, #PMO, @DG9-PMO, &MOE. *p<0.05, **p<0.01, ***p<0.005. Error bars: SEM.

FIG. 17 shows DG9-PMO maintains SMN levels in SMA mice at postnatal day 30. Representative images from western blotting and the quantification of SMN levels. The tissues were collected at PD30 from 40 mg/kg groups, relative to β-Tubulin. The heterozygous mice were used as a control with the relative SMN expression set to 1. Statistics performed using one-way ANOVA followed by post hoc Tukey's test. #PMO, @DG9-PMO, &MOE. *p<0.05, **p<0.01,***p<0.005. Error bars: SEM.

FIG. 18 shows muscle strength and motor function tests in DGO-PMO treated adult SMA mice. (A) Forelimb grip strength measured in adult males and females at PD30. 40 mg/kg of AOs were injected at PD0. (B) Forelimb grip strength normalized to the body weight. Measured at PD30 and PD60. 80 mg/kg of AOs were injected at PD0. (C) Rotarod test with an acceleration profile. Measured at PD 30-35. Each mouse was subjected to three trials spaced 20 minutes from one other. The maximum time on the beam was noted down. 40 mg/kg of AOs were injected at PD0. One-way ANOVA followed by post hoc Tukey's test was performed *NT, #PMO, @DG9-PMO, &MOE. *p<0.05, **p<0.01, ***p<0.005. Error bars: SEM.

FIG. 19 shows DG9-PMO improves the muscle pathology in SMA mice at postnatal day 7. Frequency distribution of cross-sectional area (CSA) (μm²) and the quantification of individual myofibers from the quadriceps muscle, diaphragm and intercostal muscle harvested at PD7. Box edges, 25^{th} and 75^{th} percentiles; central line, median; whiskers, range (n=3-7 per group), with around 1292-1653 fibers for the quadriceps, 917-1746 fibers for the diaphragm and 642-1127 for the intercostal muscle. Statistics was performed using one-way ANOVA followed by post hoc Tukey's test. *NT, #PMO, @9DG-PMO, &MOE. *p<0.05, **p<0.01, ***p<0.005. Error bars: SEM.

FIG. 20 shows muscle pathology of DG9-PMO treated mice at postnatal day 30. (A) Representative images from H & E staining of the quadriceps muscle (top row), diaphragm (middle row) and the intercostal muscle (bottom row) at PD30 in the heterozygous, NT control and treated groups. 40 mg/kg of AOs were injected at PD0. Scale bar: 100 μm. (B) Frequency distribution of the minimal Feret's diameter (μm) and the quantification of individual myofibers from tissues collected at PD30 shown below. (C) Frequency distribution of cross-sectional area (CSA) (μm²) and the quantification of individual myofibers shown below. Box edges, 25^{th} and 75^{th} percentiles; central line, median; whiskers, range (n=3-6 per group), with around 960-1868 fibers for the quadriceps, 983-1436 fibers for the diaphragm and 800-1802 for the intercostal muscle. 40 mg/kg of AOs were injected at PD0. Statistics was performed using one-way ANOVA followed by post hoc Tukey's test. #PMO, @DG9-PMO, &MOE. *p<0.05, **p<0.01, ***p<0.005. Error bars: SEM.

FIG. 21 shows DG09-PMO treatment does not lead to an apparent immune response and toxicity. (A) Representative images from immunostaining of D068⁺ macrophages (green) and DAPI (blue) in the quadriceps muscle at PD7. (B) Average CD068⁺ cells per section. 3-5 sections from the quadriceps muscle from each mouse were used for analysis. (n=3-5 per group). scale bar: 100 μm. (C) Serum analysis for ALP, ALT, AST, total bilirubin, GGT, BUN, creatinine, CK, total protein, albumin, and globulin. Serum was collected at PD30. (n=2-7 per group). (D) Representative images from H and E staining of the liver (top row) and kidney (bottom row) at PD30. No apparent morphological differences were observed between the healthy heterozygous control and treated groups. scale bar: 100 μm. 40 mg/kg of AOs were injected at PD0. One-way ANOVA followed by post hoc Tukey's test was performed. *NT, #PMO, @DG9-PMO, &MOE. *p<0.05, **p<0.01,***p<0.005. Error bars: SEM.

FIG. 22 shows a DG9 PMO induced heart-specific exon-skipping in a transgenic zebrafish model. (A) A transgenic zebrafish line is produced using the Tol2 transposon system by flanking the dual fluorescent protein switch with Tol2 inverted terminal repeats (ITR). Ubiquitous mRNA is produced using the carp beta-actin promoter, non-coding exon, and mini intron 1 (ß-act). mRNA will be produced from two exons. The first exon contains a complete blue fluorescent protein (BFP) open reading frame including a stop codon. The second exon includes a red fluorescence protein (RFP) open reading frame with its stop codon, a polyadenylation signal and a transcription terminator. This dual cistronic mRNA will normally only produce functional BFP. However, in the presence of a PMO targeted against the splice acceptor of the BFP (derived from exon 2 of the carp beta actin gene), the BFP exon will be skipped, and a shorter mRNA will be made. RFP can be translated from this shorter mRNA. The switch from expression of BFP to RFP will correspond with PMO activity in the nucleus and indicates successful delivery into the cells. B. qPCR data shows detection of the RFP transcript collected from the DG9 PPMO-treated zebrafish group (15 months old, ˜8 fish per group, injected with 1 dose of 25 mg/Kg PPMO via an intravenous route). ACT plots of RFP transcript: out of all the organs tested, the heart was the only organ that showed viable detection of the RFP. C. Gel electrophoresis was run on the qPCR product to visualize RFP transcript. The ˜100 bp band detected in the heart was sequenced and the result of which was aligned with the desired product.

FIG. 23 shows improved efficacy following DG9-PMO treatment compared to R6-PMO in SMA mice. (A) Survival curves of heterozygous mice (Het), non-treated (NT), unconjugated-PMO (PMO), DG9-PMO, MOE and R6G-PMO injected at PD0 at a dose of 40 mg/kg. n=15 (Hets), n=22 (NT), n=49 (unconjugated PMO), n=14 (DG9-PMO), n=29 (MOE), and n=6 (R6G-PMO), (p≤0.0001, log-rank Mantel Cox test). (B) A representative image of heterozygous mouse, mice treated with either 80 mg/kg or 40 mg/kg of DG9-PMO, mouse injected with unconjugated-PMO (40 mg/kg), and saline-treated NT mouse at PD7 (left-to-right). (C) Weight of mice at PD7 administered with either 40 or 80 mg/kg doses. Each dot (symbol) indicates a neonatal pup. (D) Hindlimb suspension assay (HLS). Mice were treated with 40 mg/kg AOs at PD0. Scored is based on the position of the hindlimbs when suspended from a falcon tube. (E) Righting reflex test. Mice were treated with 40 mg/kg AOs at PD0. The ability of mice to right themselves on their paws was measured every alternate day between PD2 to PD20 (left). The mean righting reflex time at PD6 and PD8 was also indicated (right: box whiskers plots). Box edges, 25^{th} and 75^{th} percentiles; central line, median; whiskers, range. (F) Relative expression levels of full length SMN2 (FL-SMN2) compared to deleted SMN2 transcripts (Δ7 SMN2) measured by qPCR following 40 mg/kg ASO or PBS treatments. In C, E (box whisker plots), and F, One-way ANOVA followed by post hoc Tukey's test was performed. *p<0.05, **p<0.01, ***p<0.005. In D and E, Two-way ANOVA followed by Sidak's multiple comparison was performed. *p<0.03; **p<0.002; ***p<0.0002.*NT, #PMO, @DG9-PMO, &MOE, \hat{ }R6-PMO. Error bars: SEM

### DETAILED DESCRIPTION

The invention provides pre-mRNA splicing modulating therapeutics optionally suitable for systematic delivery. The therapeutics comprise antisense oligonucleotides conjugated to a cell penetrating peptide derived from the protein transduction domain (PTD) of the human Hph-1 transcription factor.

The therapeutics are designed to either promote exon skipping or exon inclusion. The therapeutics includes peptide-conjugated antisense therapeutics for the treatment of Duchenne Muscular Dystrophy (DMD) and Spinal Muscular Atrophy (SMA).

DMD therapeutics promote exon skipping. Optionally such therapeutics comprise more than one conjugate and are configured to promote multi-exon skipping. Optionally, the therapeutic comprises two, three, four, five, six, seven or more different conjugates wherein each conjugate comprises antisense oligonucleotides conjugated to a cell penetrating peptide derived from the protein transduction domain (PTD) of the human Hph-1 transcription factor. In some embodiments, each different conjugate targets a different exon. In some embodiments, different conjugates target the same exon.

SMA therapeutics promote exon inclusion.

### Conjugates:

Conjugates of the invention comprise an antisense oligonucleotide capable of modulating exon splicing of pre-mRNA attached to a cell penetrating peptide (CPP). The cell penetrating peptide of the invention, also referred to as DG9, facilitates the delivery of the conjugated antisense oligonucleotide and has improved activity over the R6G cell penetrating peptide. In some embodiments, conjugates of the invention are for intravenous delivery.

The cell penetrating peptide of the invention is derived from the protein transduction domain of human HPH-1 transcription factor and comprises the amino acid sequence:

YARVRRRGPRGYARVRRRGPRR.

Optionally, in some embodiments, one or two amino are deleted or substituted. Optionally, amino acids are substituted with non-naturally occurring amino acids.

In some embodiments, to increase serum stability one or more L-amino acids are replaced with D-amino acids. In some embodiments, two, three, four, five or six L-amino acids are replaced with D-amino acids. Optionally, two, three, four, five or six L-arginines are replaced with D-arginines. In some embodiments, the cell penetrating peptide (CPP) has the amino acid sequence:

YArVRRrGPRGYArVRRrGPRr;

wherein uppercase represent L-amino acids and lowercase represent D-amino acids.

In some embodiments, the CPP is conjugated to the 5′ end of the oligonucleotide. In other embodiments, the OPP is conjugated at the 3′ of the oligonucleotide.

As used herein interchangeably, “antisense oligonucleotides”, “antisense therapeutics”, “AOs”, “oligos”, “oligomers” refer to a sequence of subunits, each having a base carried on a backbone subunit, and where the backbone groups are linked by intersubunit linkages that allow the bases in the compound to hybridize to a target sequence in a nucleic acid by Watson-Crick base pairing, to form a nucleic acid:oligonucleotide heteroduplex within the target sequence. The oligonucleotides may have exact sequence complementarity to the target sequence or sufficient complementarity to selectively bind the target sequence.

The antisense oligonucleotide are between about 20 to about 50 nucleotides in length, including at least 10, 12, 15, 17, or 20 consecutive nucleotides of complementary to the target sequence.

In one embodiment, the antisense oligonucleotide is 20 to 30 or 24 to 28 nucleotides in length.

In some embodiments, the antisense oligonucleotide is an antisense oligonucleotide analogue.

The term ‘oligonucleotide analogue’ and ‘nucleotide analogue’ refers to any modified synthetic analogues of oligonucleotides or nucleotides respectively that are known in the art.

Examples of oligonucleotide analogues include, but are not limited to, peptide nucleic acids (PNAs), morpholino oligonucleotides, phosphorothioate oligonucleotides, phosphorodithioate oligonucleotides, alkylphosphonate oligonucleotides, acylphosphonate oligonucleotides, phosphoramidite oligonucleotides, tricyclo-DNA, and 2′methoxyethyl oligonucleogides.

In some embodiments, the antisense oligonucleotide comprises morpholino subunits.

In some embodiments, the antisense oligonucleotide is a morpholino antisense oligonucleotide.

In some embodiments, the antisense oligonucleotide comprises morpholino subunits linked together by phosphorus-containing linkages. In a specific example, the antisense oligonucleotide is a phosphoramidate or phosphorodiamidate morpholino antisense oligonucleotide.

The terms ‘morpholino antisense oligonucleotide’ or ‘PMO’ (phosphoramidate or phosphorodiamidate morpholino oligonucleotide) refer to an antisense oligonucleotide analog composed of morpholino subunit structures, where (i) the structures are linked together by phosphorus-containing linkages, for example one to three atoms long, for example two atoms long, and for example uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide.

In some embodiments, the antisense oligonucleotide comprises phosphorus-containing intersubunit linkages joining a morpholino nitrogen of one subunit to a 5′ exocyclic carbon of an adjacent subunit.

Optionally, variations can be made to the intersubunit linkage as long as the variations do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to the phosphorus may be unsubstituted, monosubstituted, or disubstituted with (optionally substituted) lower alkyl.

Antisense oligonucleotide may be made through the well-known technique of solid phase synthesis.

In some embodiments, the conjugate further comprises a label. The label provides a direct or indirect detectable signal. Appropriate labels are known in the art and include fluorescent or radioactive labels.

Methods of conjugating the CPP to the oligonucleotide are known in the art or the conjugate can be prepared commercially.

### Duchenne Muscular Dystrophy (DMD) Conjugates

There are provided exon skipping peptide-conjugated antisense therapeutics for the treatment of Duchenne Muscular Dystrophy. Treatment of DMD with peptide-conjugated antisense therapeutics restores partially functional dystrophin to the DMD patient.

A ‘functional’ dystrophin protein refers to a dystrophin protein having sufficient biological activity to reduce the progressive degradation of muscle tissue that is otherwise characteristic of muscular dystrophy when compared to the defective form of dystrophin protein that is present in subjects with a muscular disorder such as DMD.

In some embodiments, a functional dystrophin protein may have about 10%, 20%, 30%, 40%, 50%, 60%, 70%, 80%, 90%, or 100% of the in vitro or in vivo biological activity of wild-type dystrophin. The activity of dystrophin in muscle cultures in vitro can be measured according to myotube size, myofibril organization, contractile activity, and spontaneous clustering of acetylcholine receptors.

Accordingly, in some embodiments, the peptide-conjugated antisense therapeutics of the invention are for use in the treatment of DMD by inducing exon skipping in the human dystrophin pre-mRNA to restore functional dystrophin protein expression.

In one embodiment, the therapeutic comprises one peptide-conjugated antisense oligonucleotide. In alternative embodiments, the therapeutic comprises more than one peptide conjugated antisense oligonucleotide, wherein each peptide conjugated antisense oligonucleotide targets a different sequence in the dystrophin pre-mRNA.

As described herein, there is provided peptide-conjugated antisense therapeutics that may be therapeutically effective for exon skipping therapy skipping in the human dystrophin (DMD) gene, of one or more of exon 44, exon 45, exon 46, exon 47, exon 48, exon 49, exon 50, exon 51, exon 52, exon 53, exon 54 and/or exon 55.

In some embodiments, the peptide-conjugate antisense therapeutic comprises a plurality of peptide-conjugated antisense oligonucleotides and promotes skipping of exons 45-55.

In some embodiments, the peptide-conjugate antisense therapeutic comprises a peptide-conjugated antisense oligonucleotide targeting exon 45, a peptide-conjugated antisense oligonucleotide targeting exon 46, a peptide-conjugated antisense oligonucleotide targeting exon 47, a peptide-conjugated antisense oligonucleotide targeting exon 48, a peptide-conjugated antisense oligonucleotide targeting exon 49, a peptide-conjugated antisense oligonucleotide targeting exon 50, a peptide-conjugated antisense oligonucleotide targeting exon 51, a peptide-conjugated antisense oligonucleotide targeting exon 52, a peptide-conjugated antisense oligonucleotide targeting exon 53, a peptide-conjugated antisense oligonucleotide targeting exon 54 and a peptide-conjugated antisense oligonucleotide targeting exon 55.

In other embodiments, the peptide-conjugate antisense therapeutic comprises a peptide-conjugated antisense oligonucleotide targeting exon 45, a peptide-conjugated antisense oligonucleotide targeting exon 47, a peptide-conjugated antisense oligonucleotide targeting exon 49, a peptide-conjugated antisense oligonucleotide targeting exon 51, a peptide-conjugated antisense oligonucleotide targeting exon 53 and a peptide-conjugated antisense oligonucleotide targeting exon 55.

In still other embodiments, the peptide-conjugate antisense therapeutic comprises a peptide-conjugated antisense oligonucleotide targeting exon 45, a peptide-conjugated antisense oligonucleotide targeting exon 47 and a peptide-conjugated antisense oligonucleotide targeting exon 53.

In some embodiments, the antisense oligonucleotide of the peptide-conjugated antisense therapeutics binds to a target sequence in exon 44, exon 45, exon 46, exon 47, exon 48, exon 49, exon 50, exon 51, exon 52, exon 53, exon 54 or exon 55 of the human dystrophin (DMD) gene. Optionally, the target sequence is proximal to the exon acceptor splice site in the pre-mRNA. In some embodiments, the target sequence is at the exon acceptor splice site, within 10 bases of the exon splice site, within 15 bases of the exon splice site, within 20 bases of the exon splice site, within 30 bases of the exon splice site, within 40 bases of the axon splice site, within 50 bases of the exon splice site, within 75 bases of the axon splice site or within 100 bases of the exon splice site

In some embodiments, the anti-sense oligonucleotide of the conjugate comprises or consists of at least 10, 12, 14, 16, 18 or 20 consecutive nucleotides of any one of the following sequences wherein, thymine bases in the sequences are optionally uracil:

| ID* | Sequence, 5′ to 3′ |
|---|---|
| Ex45_Ac9 | GACAACAGTTTGCCGOTGCCCAATGCCATC |
| Ex46_Ac93 | AGTTGCTGCTCTTTTCCAGGTTCAAGTGGG |
| Ex47_Ac13 | GTTTGAGAATTCCCTGGCGCAGGGGCAACT |
| Ex48_Ac7 | CAATTTCTCCTTGTTTCTCAGGTAAAGCTC |
| Ex48_Ac78 | CAGATGATTTAACTGCTCTTCAAGGTCTTC |
| Ex49_Ac17 | ATCTCTTCCACATCCGGTTGTTTAGOTTGA |
| Ex50_Ac19 | GTAAACGGTTTACCGCCTTCCACTCAGAGC |
| Ex51_Ac0 | GTGTCACCAGAGTAACAGTCTGAGTAGGAG |
| Ex52_Ac24 | GGTAATGAGTTCTTCCAACTGGGGACGCCT |
| Ex53_Ac26 | CCTCCGGTTCTGAAGGTGTTCTTGTACTTC |
| Ex54_Ac42 | GAGAAGTTTCAGGGCCAAGTCATTTGCCAC |
| Ex55_Ac0 | TCTTCCAAAGCAGCCTCTCGCTCACTCACC |

*represented as: exon target_distance from target exon acceptor site.

Optionally, the antisense oligonucleotide is a phosphorodiamidate morpholino oligomer (PMO).

In some embodiments, the conjugates have increased delivery to, uptake or retention by cardiac cells.

### Spinal Muscular Atrophy (SMA)

There are provided exon inclusion peptide-conjugated antisense therapeutics for the treatment of Spinal Muscular Atrophy. Treatment of SMA with the peptide-conjugated antisense therapeutics results in the production of stable SMN2 to compensate for the loss of SMN1.

The peptide-conjugated antisense therapeutics comprises DG-9 conjugated to an antisense oligonucleotide that binds to intronic splicing silencer N1 of SMN2 pre-mRNA. Optionally, the antisense oligonucleotide comprises or consists of the sequence 5′-TCACTTTCATAATGCTGG-3′ and wherein the thymines are optionally replaced with uracil.

In some embodiments, the antisense oligonucleotide is a phosphorodiamidate morpholino oligomer.

### Pharmaceutically Acceptable Excipient

In one aspect there is described a pharmaceutical composition comprising the antisense oligonucleotide(s) of the invention or a conjugate thereof, further comprising one or more pharmaceutically acceptable excipients.

In some embodiments, the pharmaceutical composition is prepared in a manner known in the art, with pharmaceutically inert inorganic and/or organic excipients being used.

The term ‘pharmaceutically acceptable’ refers to molecules and compositions that are physiologically tolerable and do not typically produce an allergic or similarly untoward reaction when administered to a patient.

In some embodiments, the pharmaceutical composition may be formulated as a pill, tablet, coated tablet, hard gelatin capsule, soft gelatin capsule and/or suppository, solution and/or syrup, injection solution, microcapsule, implant and/or rod, and the like.

In some embodiments, the pharmaceutical composition may be formulated as an injection solution.

In some embodiments, pharmaceutically acceptable excipients for preparing pills, tablets, coated tablets and hard gelatin capsules may be selected from any of: Lactose, corn starch and/or derivatives thereof, talc, stearic acid and/or its salts, etc.

In some embodiments, pharmaceutically acceptable excipients for preparing soft gelatin capsules and/or suppositories may be selected from fats, waxes, semisolid and liquid polyols, natural and/or hardened oils, etc.

In some embodiments, pharmaceutically acceptable excipients for preparing solutions and/or syrups may be selected from water, sucrose, invert sugar, glucose, polyols, etc.

In some embodiments, pharmaceutically acceptable excipients for preparing injection solutions may be selected from water, saline, alcohols, glycerol, polyols, vegetable oils, etc.

In some embodiments, pharmaceutically acceptable excipients for preparing microcapsules, implants and/or rods may be selected from mixed polymers such as glycolic acid and lactic acid or the like.

In some embodiments, the pharmaceutical composition may comprise a liposome formulation.

In some embodiments, optionally, the pharmaceutical composition may comprise two or more different antisense oligonucleotides or conjugates thereof.

In some embodiments, optionally, the antisense oligonucleotide and/or conjugate may be present in the pharmaceutical composition as a physiologically tolerated salt.

Suitably, physiologically tolerated salts retain the desired biological activity of the antisense oligonucleotide and/or conjugate thereof and do not impart undesired toxicological effects. For antisense oligonucleotides, suitable examples of pharmaceutically acceptable salts include (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

In some embodiments, in addition to the active ingredients and excipients, a pharmaceutical composition may also comprise additives, such as fillers, extenders, disintegrants, binders, lubricants, wetting agents, stabilizing agents, emulsifiers, preservatives, sweeteners, dyes, flavorings or aromatizing agents, thickeners, diluents or buffering substances, and, in addition, solvents and/or solubilizing agents and/or agents for achieving a slow release effect, and also salts for altering the osmotic pressure, coating agents and/or antioxidants. Suitable additives may include Tris-HCl, acetate, phosphate, Tween 80, Polysorbate 80, ascorbic acid, sodium metabisulfite, Thimersol, benzyl alcohol, lactose, mannitol, or the like.

### Administration

In some embodiments, the peptide conjugated antisense oligonucleotide(s) and/or pharmaceutical composition is for topical, enteral or parenteral administration.

In some embodiments, the peptide conjugated antisense oligonucleotide(s) and/or pharmaceutical composition may be for administration orally, transdermally, intravenously, intrathecally, intramuscularly, subcutaneously, nasally, transmucosally or the like.

In some embodiments, the antisense oligonucleotide(s) and/or pharmaceutical composition is for intramuscular administration.

In some embodiments, the antisense oligonucleotide(s) and/or pharmaceutical composition is for intramuscular administration by injection.

An ‘effective amount’ or ‘therapeutically effective amount’ refers to an amount of the antisense oligonucleotide, administered to a subject, either as a single dose or as part of a series of doses, which is effective to produce a desired physiological response or therapeutic effect in the subject.

In some embodiments, the antisense oligonucleotide(s) or conjugate thereof are administered daily, once every 2, 3, 4, 5, 6, 7, 8, 9, 10, 11, 12, 13, 14 days, once every 1, 2, 3, 4, 5, 6, 7, 8, 9, 10, 11, 12 weeks, or once every 1, 2, 3, 4, 5, 6, 7, 8, 9, 10, 11, 12 months.

In some embodiments, the antisense oligonucleotide or conjugate thereof may be administered as two, three, four, five, six or more sub-doses separately at appropriate intervals throughout the day, optionally, in unit dosage forms.

To gain a better understanding of the invention described herein, the following examples are set forth. It should be understood that these examples are for illustrative purposes only. Therefore, they should not limit the scope of this invention in anyway.

### EXAMPLES

### Example 1: Peptide-Conjugated PMOS for Treatment of Duchenne Muscular Dystrophy

Duchenne muscular dystrophy (DMD) is primarily caused by out-of-frame deletions in the dystrophin gene. Exon skipping using phosphorodiamidate morpholino oligomers (PMOs) converts out-of-frame to in-frame mutations, producing partially functional dystrophin. Skipping of exons 45-55 could treat 40-47% of all patients and is associated with improved clinical outcomes. The development of peptide-conjugated PMOs for exons 45-55 skipping is reported. Experiments with immortalized patient myotubes revealed that exons 45-55 could be skipped by targeting as few as 5 exons. Conjugating DG9, a cell-penetrating peptide to PMOs improved single-exon 51 skipping, dystrophin restoration, and muscle function in hDMDdel52;mdx mice. Local administration of a minimized exons 45-55-skipping DG9-PMO cocktail restored dystrophin production.

### Results

### Minimizing the Exons 45-55 Skipping Cocktail

The number of PMOs necessary to skip DMD exons 45-55 was minimized using two strategies.

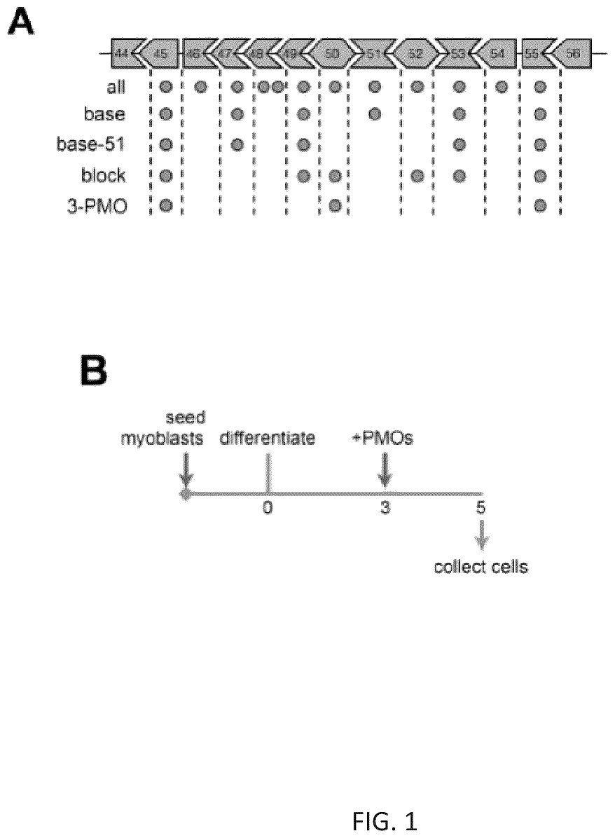

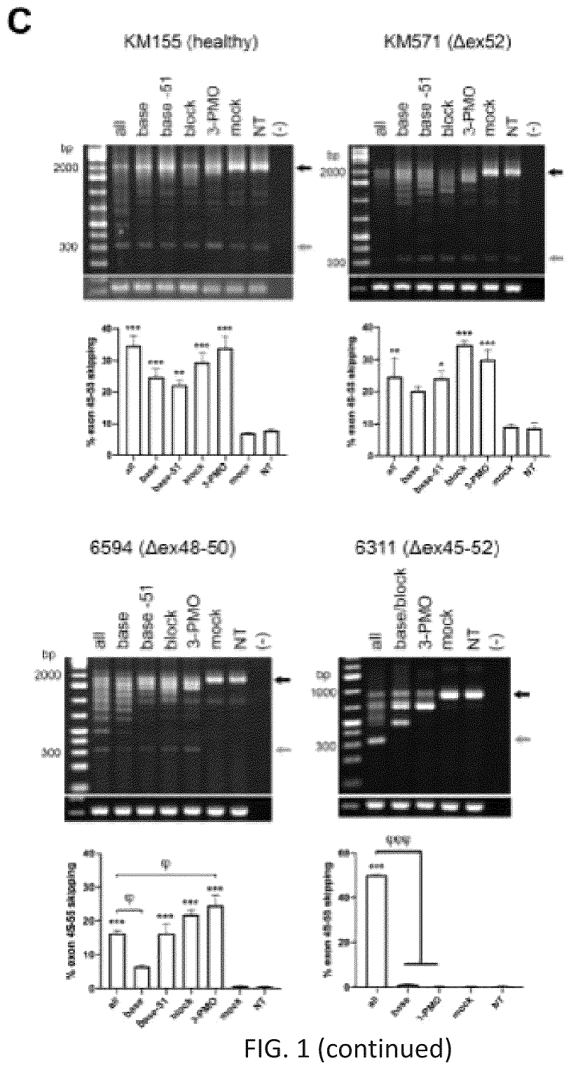

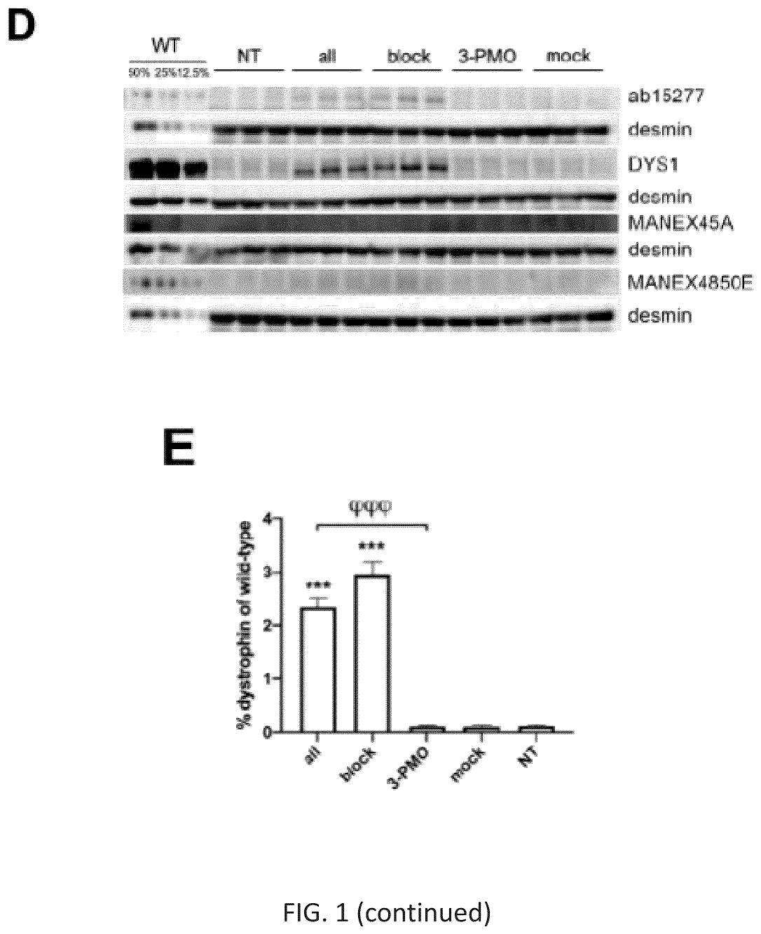

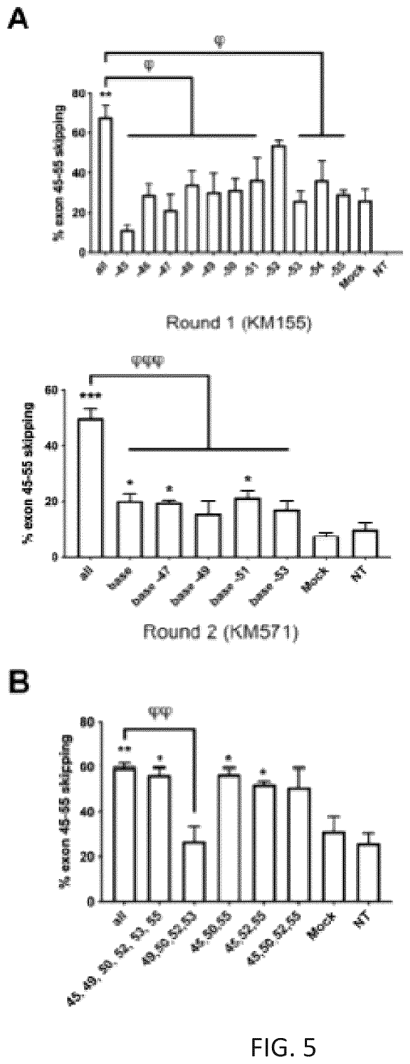

First, a PMO (or PMOs) that targeted an exon in the region were removed from the previously developed exons 45-55 skipping cocktail [13) (Table 1). The full cocktail, with PMOs targeting all exons within exons 45-55, is referred to as the “all” cocktail (FIG. 1a). Upon transfecting the “all” cocktail and its derivatives into healthy immortalized patient-derived KM155 myotubes (FIG. 1b), only the “all” cocktail skipped exons 45-55 significantly higher than the mock (p<0.005) (FIG. 5a). PMOs whose absence led to a considerable drop in skipping were kept as part of the minimized cocktail, i.e., those targeting exons 45, 47, and 53. PMOs targeting exons 49, 51, and 55 were also retained. This minimized cocktail was called the “base” cocktail, and was subjected to another round of minimization in immortalized exon 52-deleted patient-derived KM571 myotubes. The “base” cocktail showed significant exons 45-55 skipping (p<0.05) compared to the mock, as well as its derivatives where exon 47 (p<0.05) or 51 (p<0.05) were not targeted (FIG. 5a). The “base” and “base-51” cocktail showed the highest skipping efficiencies in this batch (FIG. 1a).

For the second strategy, minimized cocktails based on the endogenous splicing of DMD exons 45-55 were prepared. A model proposes that exons 45-49, 50-52, and 53-55 are spliced first, after which these groups are spliced together to complete the exons 45-55 region (25). Derivative cocktails from the “all” cocktail with PMOs targeting the terminal exons of these three groups were designed. Transfection into healthy KM155 myotubes revealed three derivative cocktails to skip exons 45-55 significantly higher than the mock (p<0.05) (FIG. 5b), with skipping efficiencies not significantly different from the “all” cocktail. Based on this, cocktails targeting exons 45, 49, 50, 52, 53, and 55 (“block” cocktail) were selected as well as targeting exons 45, 50, and 55 (“3-PMO” cocktail) for further experiments (FIG. 1a).

### Minimized Exon Skipping Cocktails Effectively Skip Exons 45-55 in Various Patient Cells

The “all”, “base” “base-51”, “block”, and “3-PMO” exons 45-55 skipping cocktails were subsequently tested in the following immortalized patient-derived muscle cell lines: KMI55 (healthy), KM571 (Δex52), 6594 (Δex48-50), and 6311 (Δex45-52) (FIG. 1a). Mutation-tailored versions of the cocktails were used for each cell line, with unnecessary PMOs removed. Upon transfection into myotubes (FIG. 1b) and RT-PCR analysis, the “all” cocktail significantly skipped exons 45-55 in all lines compared to the mock (p<0.005 or p<0.001) (FIG. 1c). The “base-51” “block”, and “3-PMO” cocktails induced significant exons 45-55 skipping in KM155, KM571, and 6594 myotubes compared to the mock (at least p<0.05); the “base” cocktail only showed significant skipping in KM155 cells (p<0.001). In 6594 myotubes, the “3-PMO” cocktail induced significantly higher skipping than the “all” cocktail. Intriguingly, none of the minimized cocktails showed any exons 45-55 skipping in 6311 myotubes.

The dystrophin restoration capabilities of the “block” and “3-PMO” cocktails in KM571 myotubes were evaluated, since these cocktails induced the highest levels of exons 45-55 skipping in this line. Western blot using antibodies against the rod (DYS1, exons 26-30) and C-terminal domains (ab15277) of dystrophin detected dystrophin upon treatment with the “all” and “block” cocktails, but not with the “3-PMO” cocktail (FIG. 1d). Quantification of the DYS1 signal showed that “block” cocktail treatment restored an average 2.94% dystrophin of wild-type levels, slightly higher than the average 2.33% dystrophin restored by the “all” cocktail and elevated compared to the 0.10% dystrophin level in the mock (p<0.001) (FIG. 1e). Western blot with the MANEX45A (corresponding to exons 45-46) and MANEX4850E (corresponding to exons 48-50) antibodies did not detect dystrophin in treated myotubes (FIG. 1d), suggesting that the dystrophin induced post-treatment likely came from exons 45-55-skipped transcripts.

Single Intravenous Treatment with DG9-PMO Induces Higher Dystrophin Production than Unconjugated PMO in the Skeletal Muscles and the Heart

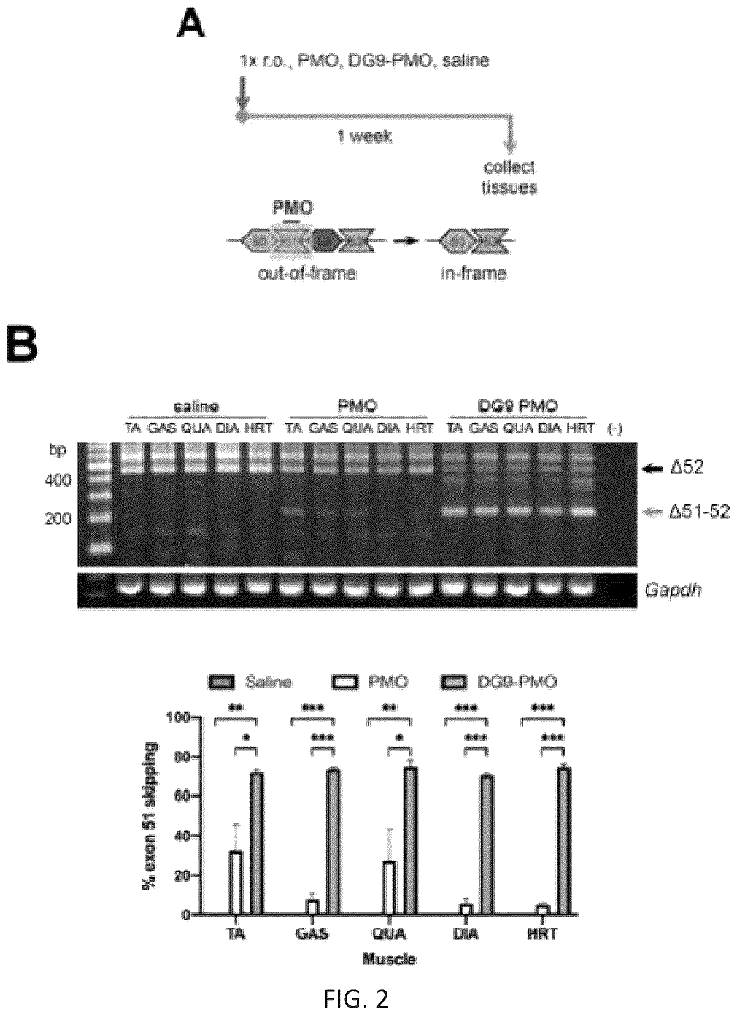

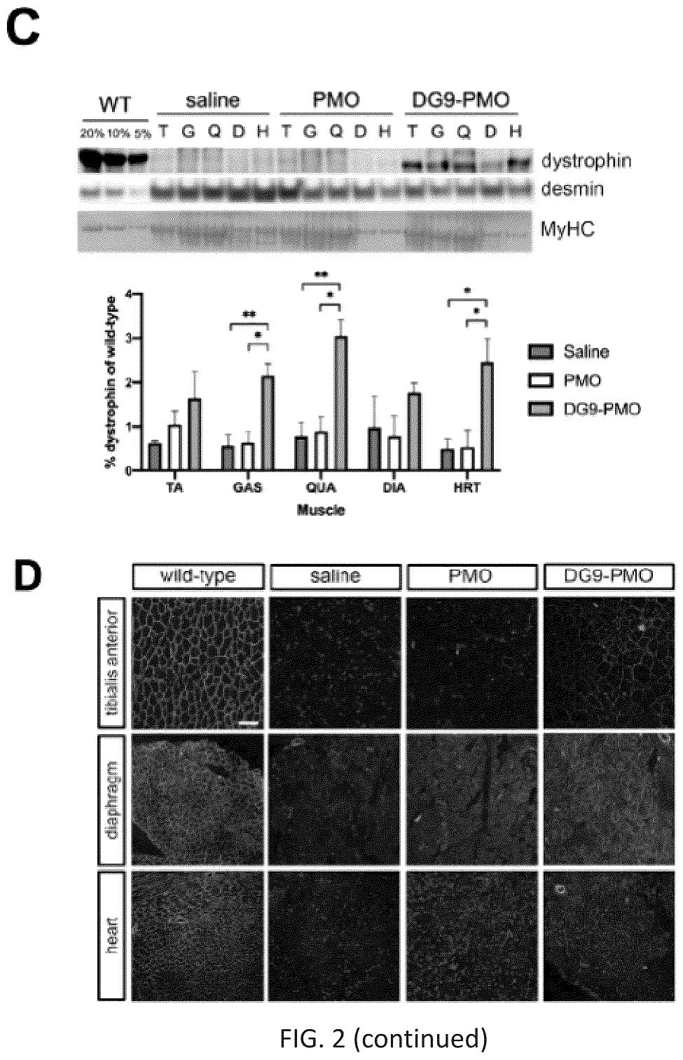

To evaluate DG9 as a conjugate for the minimized exons 45-55 skipping cocktail, its efficacy in vivo as applied to single-exon skipping was evaluated. DG9 was conjugated to Ex51_Ac0, an exon 51-skipping PMO from the “all” cocktail that was up to 7 times more effective than eteplirsen in restoring dystrophin production in vitro [13, 26). hDMDdel52;mdx mice at 3 months were given a single retro-orbital injection of either saline, 50 mg/kg unconjugated PMO, or 64 mg/kg DG9-PMO (equimolar to the PMO dose) and assessed a week later (FIG. 2a). DG9-PMO-treated mice had significantly higher levels of exon 51 skipping than the saline- or PMO-treated groups across various skeletal muscles (at least p<0.05) and the heart (p<0.001) (FIG. 2b). DG9-PMO induced 2.2 to 12.3-fold higher skipping in skeletal muscles and 14.4-fold higher skipping in the heart on average compared to unconjugated PMO. DG9-PMO treatment significantly restored dystrophin production compared to the saline (p<0.005) and PMO (p<0.05) treatments in the gastrocnemius and quadriceps, reaching up to 3% of wild-type levels (FIG. 2c). In the heart, DG9-PMO restored an average 2.5% dystrophin of wild-type levels, significantly higher than that in the saline or PMO groups (p<0.05). Compared to PMO-treated mice, mice that received DG9-PMO had 1.5 to 3.4-fold and 4.5-fold higher dystrophin protein levels on average in the skeletal muscles and heart, respectively.

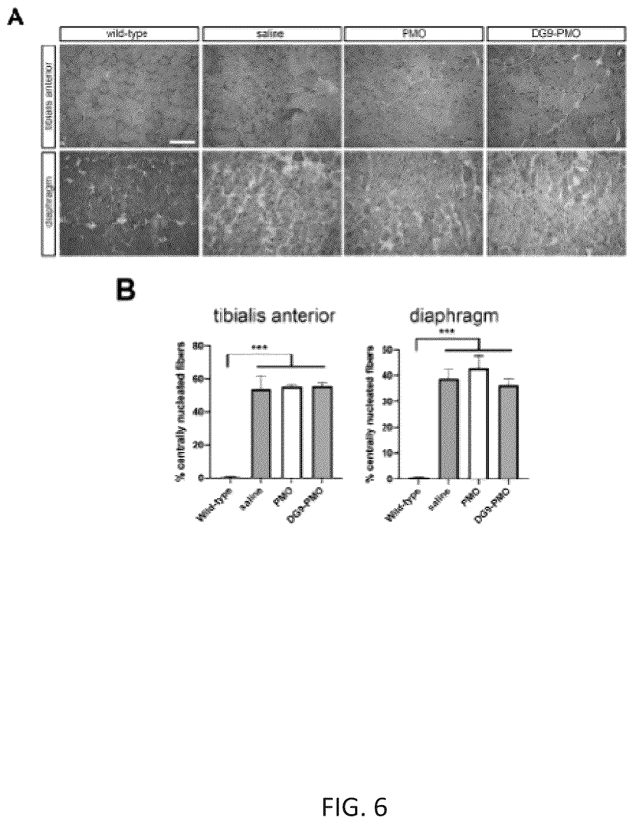

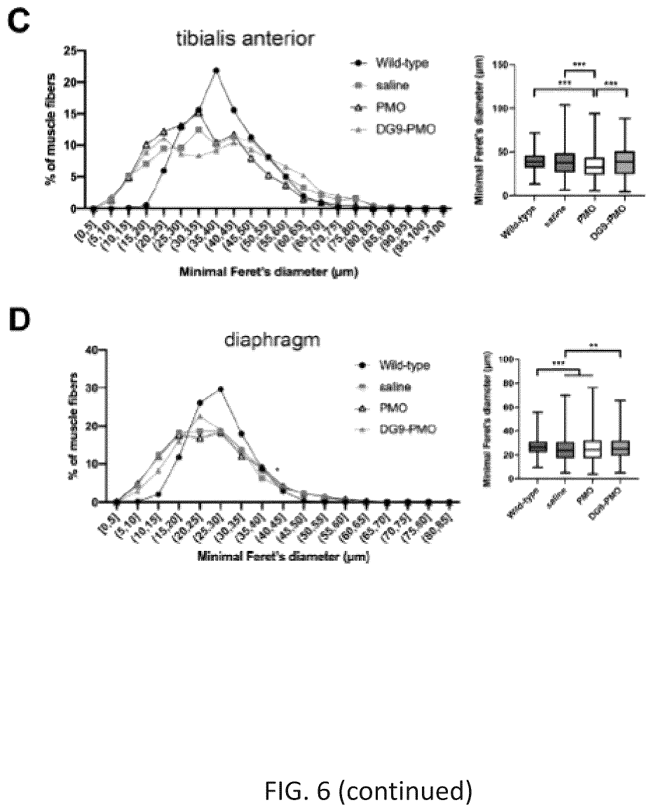

Widespread dystrophin-positive fibers were observed in the tibialis anterior, diaphragm, and heart of DG9-PMO-treated mice, and very few to none in saline- or PMO-treated mice (FIG. 2d). However, histological analysis revealed no reductions in the percentage of centrally-nucleated fibers (CNFs, a marker of cumulative muscle regeneration) in the tibialis anterior and diaphragm (FIGS. 6a,b), nor any improvements in muscle fiber size (FIGS. 6a,c,d) after PMO or DG9-PMO treatment.

Repeated Intravenous Treatment with DG9-PMO Improves Dystrophin Production, Muscle Function, and Fiber Size in Dystrophic Mice

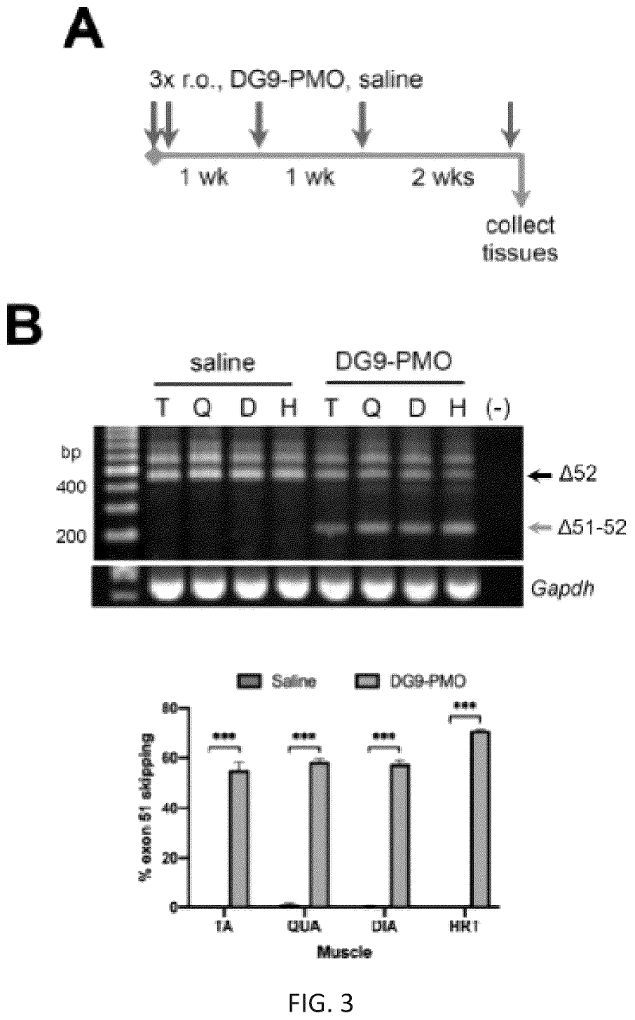

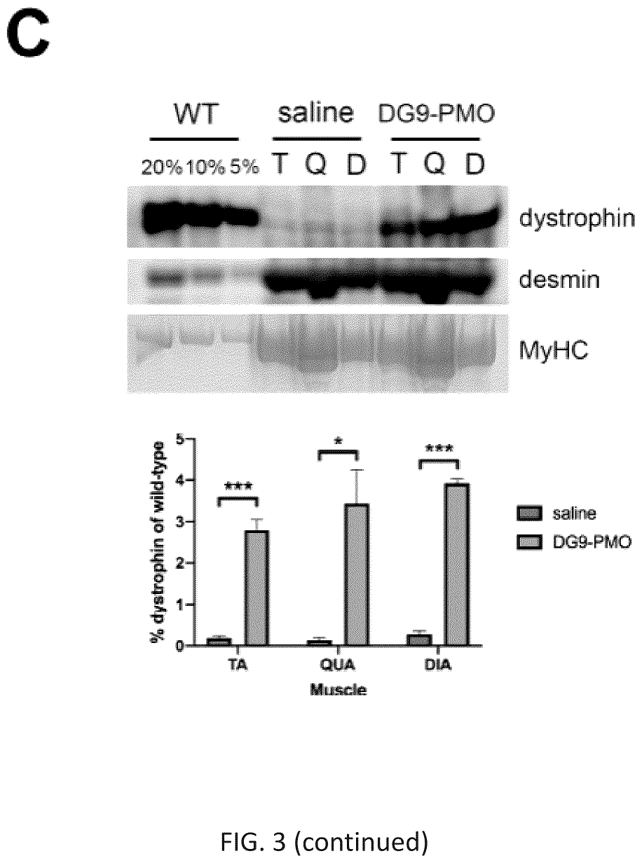

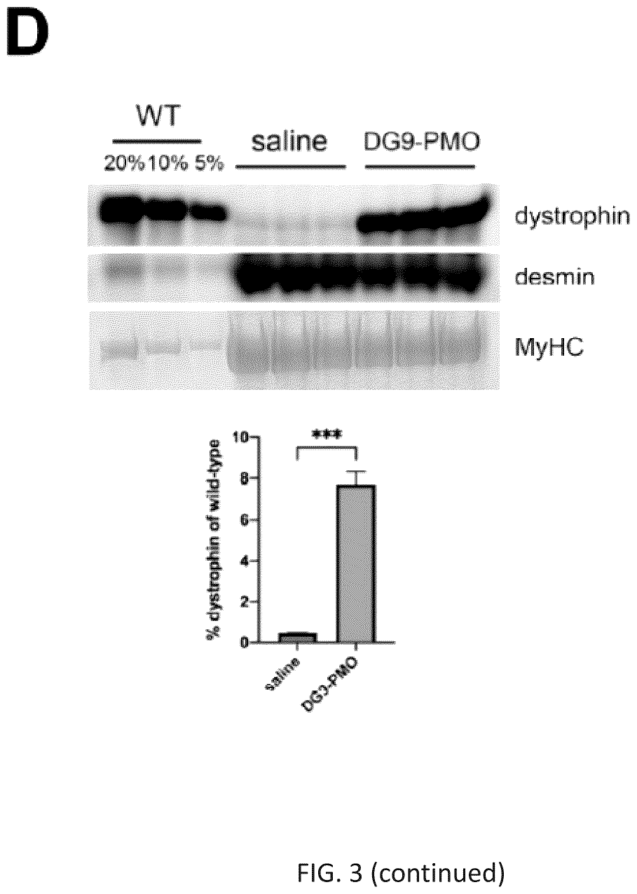

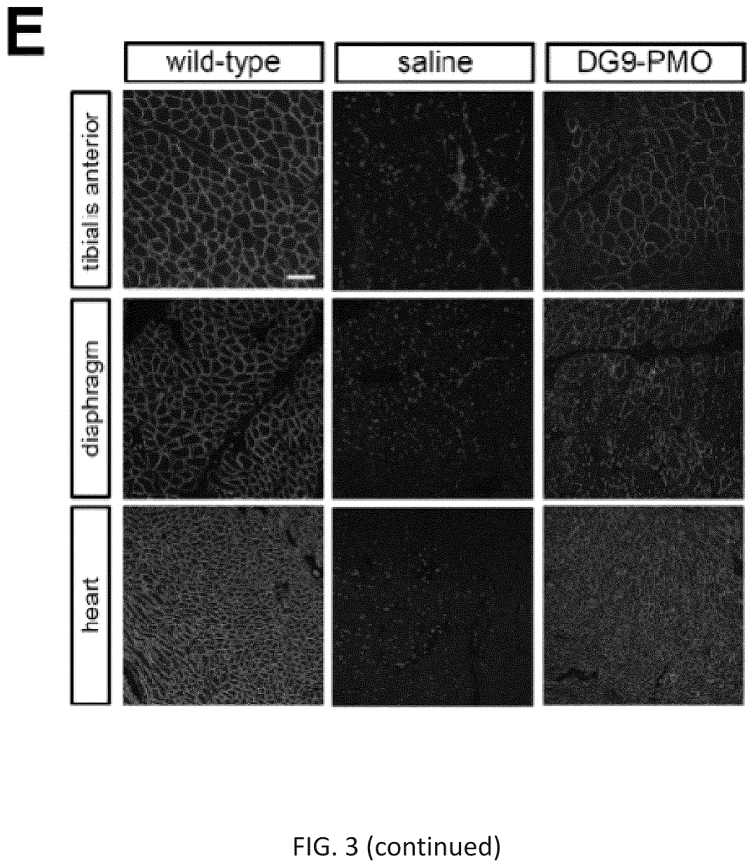

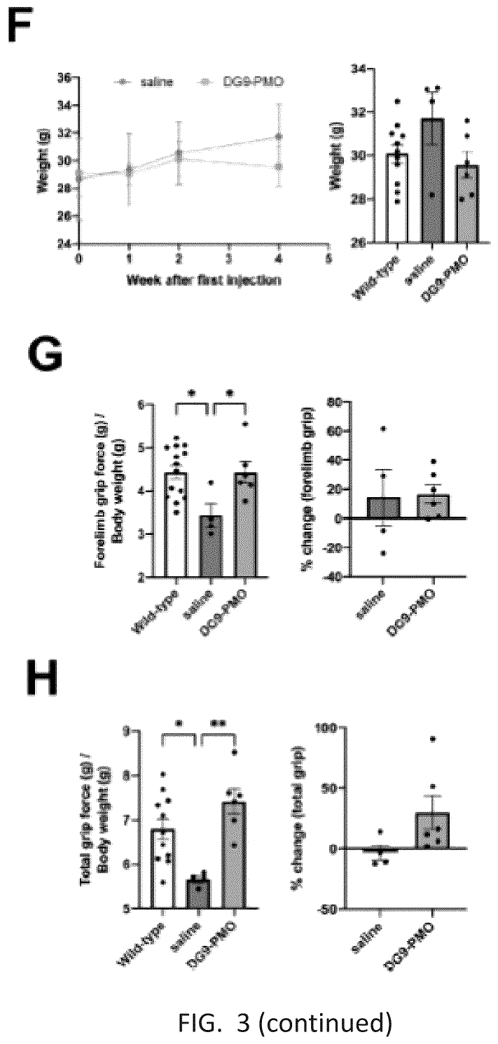

A repeated-dose treatment study was performed to provide more insight into the efficacy of DG9-PMO exon skipping therapy with regard to ameliorating dystrophic symptoms. hDMDdel52;mdx mice at 2 months were retro-orbitally injected with saline or 30 mg/kg of DG9-PMO, once weekly for 3 weeks. Functional assessments were done at baseline and at 2 weeks following the last injection, after which tissues were collected (FIG. 3a). Once again, DG9-PMO treatment significantly induced exon 51 skipping at high levels across skeletal muscles and the heart (55-71% on average) compared to the saline control (p<0.001) (FIG. 3b). This resulted in significant dystrophin production in various skeletal muscles ranging at an average of 2.8-3.9% compared to 01-0.3% in saline-treated mice (at least p<0.05) (FIG. 3c), as well as in the heart at an average of 7.7% versus 0.5% in the saline group (p<0.001) (FIG. 3d). Immunofluorescence confirmed the presence of widespread dystrophin-positive fibers in the tibialis anterior, diaphragm, and heart (FIG. 3e).

Body weights between groups did not significantly differ over the course of treatment (FIG. 3f).

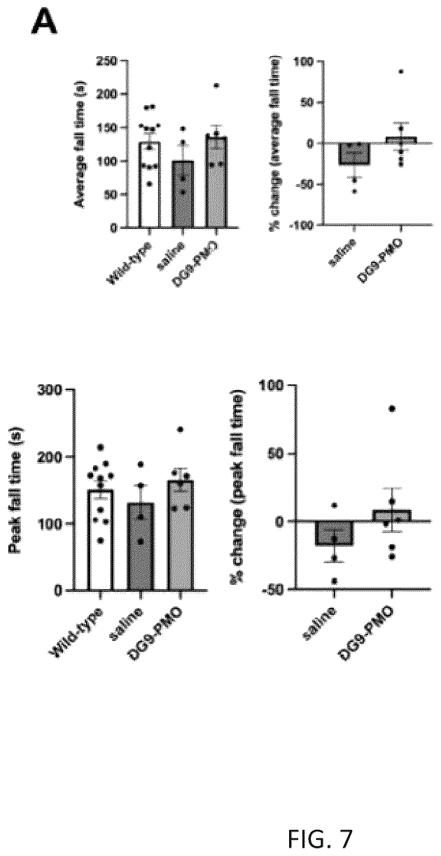

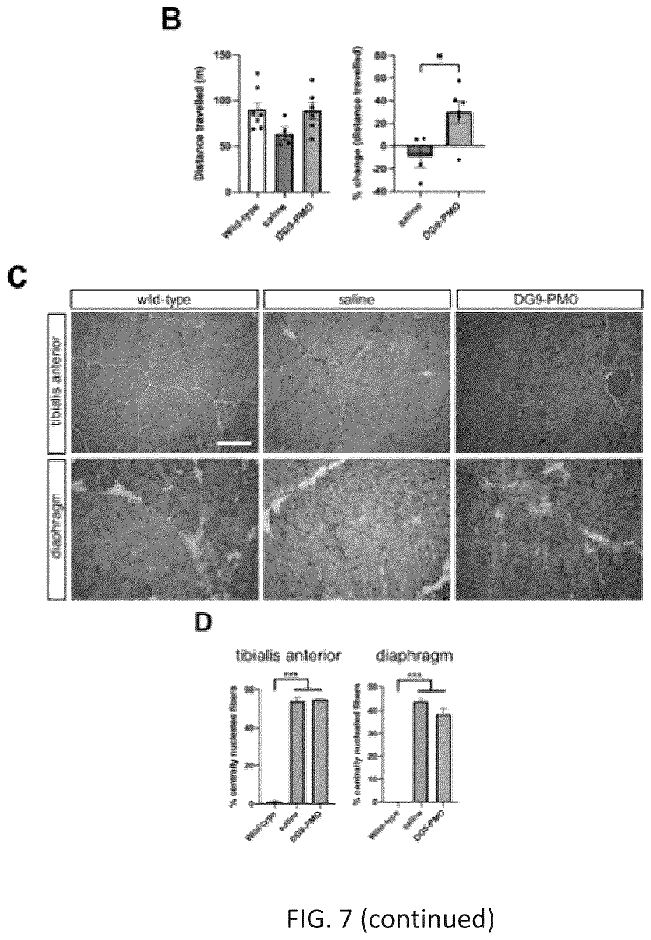

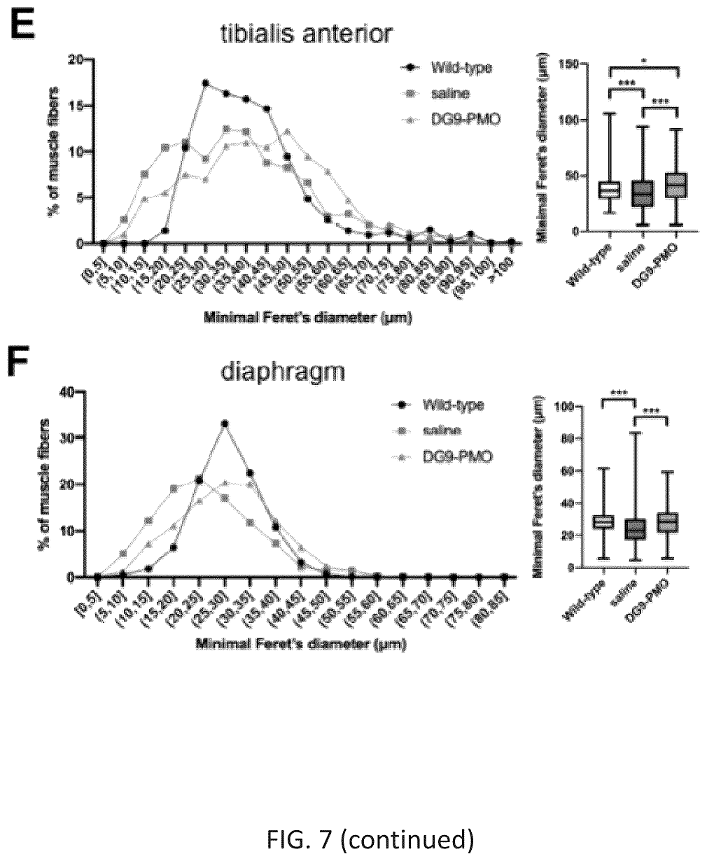

Most impressively, repeated DG9-PMO treatment significantly improved forelimb (p<0.05) (FIG. 3g) and total limb (p<0.005) (FIG. 3h) grip strength in hDMDdel52;mdx mice, such that they were not significantly different from wild-type controls. Nearly all mice had improved forelimb/total limb grip strength from baseline, except one which showed a −0.3% difference in forelimb grip strength post-treatment. This is in contrast to the saline controls, which showed no direction of change (FIG. 3g) or no change from baseline (FIG. 3h). Rotarod and treadmill tests showed similar improvements; in particular, the treadmill distance travelled significantly improved from baseline in treated versus saline control mice (FIGS. 7a,b).

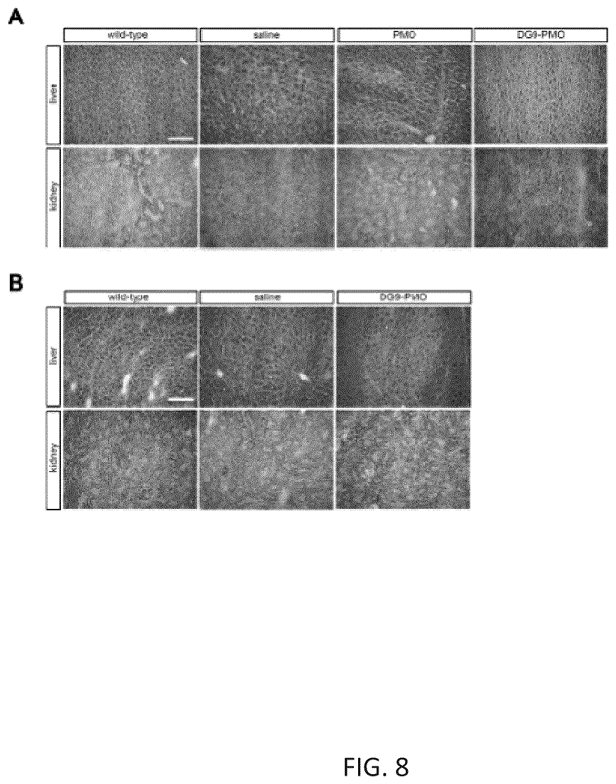

Histological analysis still did not show any significant reductions in CNFs in the tibialis anterior and diaphragm (FIGS. 7c,d). However, a significant increase in fiber size with DG9-PMO treatment (p<0.001) was observed (FIGS. 7e,f), with most fibers having a minimum Feret's diameter of 45-50 μm in the tibialis anterior and 25-30 μm in the diaphragm. This is in contrast to most fibers having diameters of 30-35 μm and 20-25 μm in saline control muscles, respectively. A qualitative histological analysis of the liver and kidney in single- and repeated-dose treatment mice was performed, but found no evidence of toxicity due to PMO or DG9-PMO treatment (FIGS. 8a,b).

Local Treatment with the DG9-Conjugated Minimized Exons 45-55 Skipping Cocktail Induces Successful Skipping and Dystrophin Production

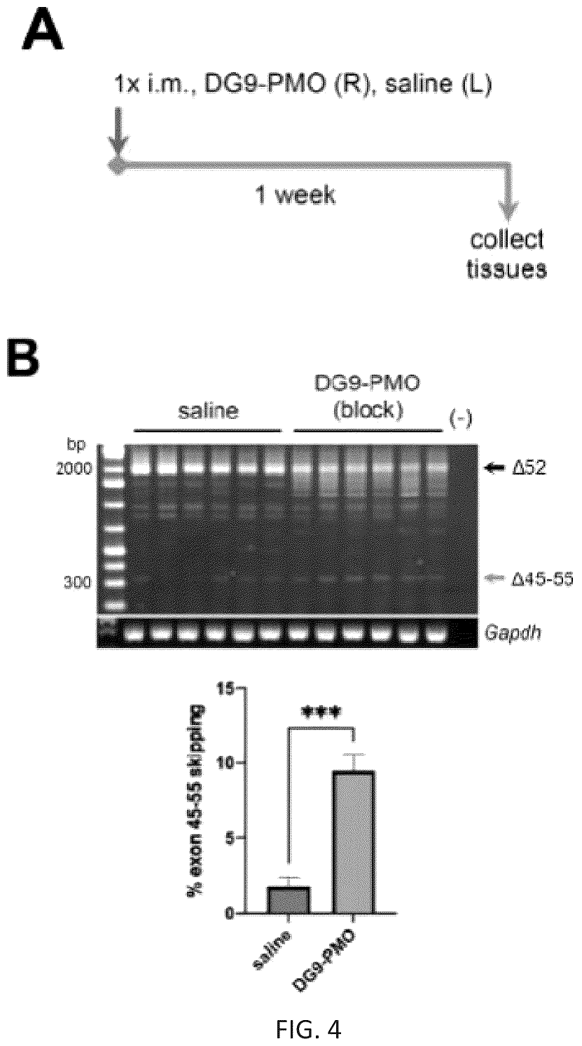

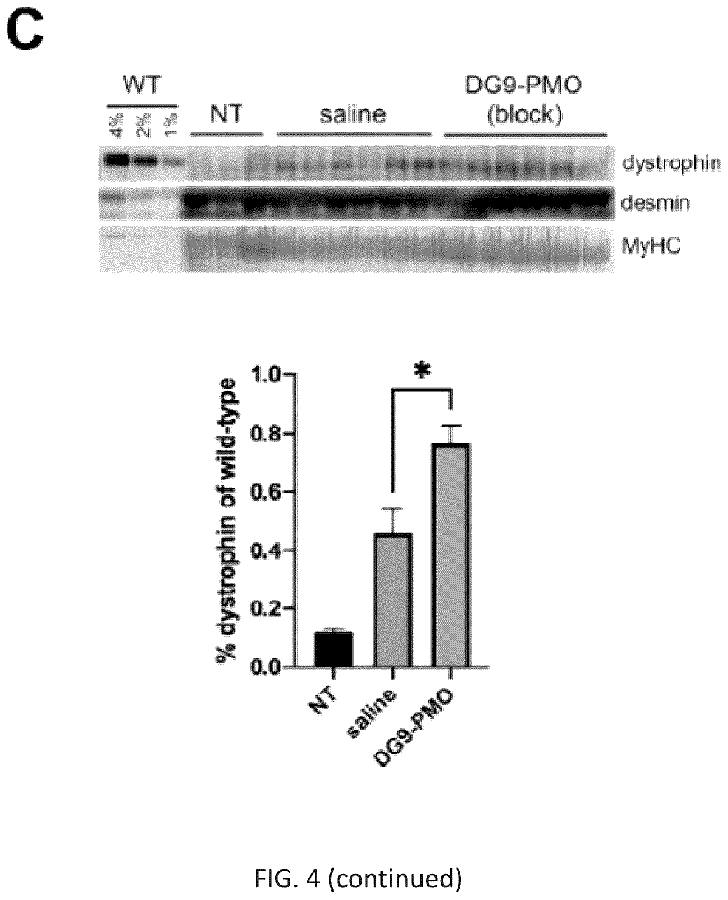

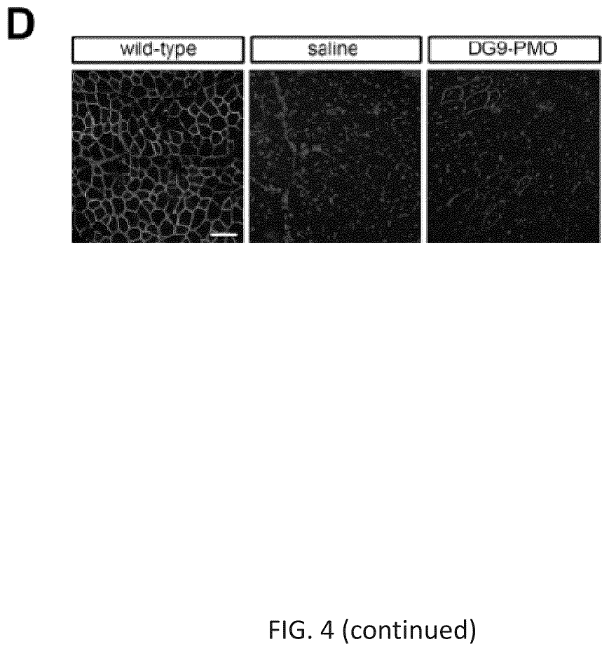

Finally, DG9 was conjugated to each PMO of the minimized “block” exons 45-55 skipping cocktail for in vivo testing. The “block” cocktail was chosen as it induced high levels of exons 45-55 skipping and dystrophin protein restoration in KM571 myotubes (FIGS. 1c-e). Thus, 5- to 6-month-old hDMDdel52;mdx mice were intramuscularly injected in the tibialis anterior with either saline or the mutation-tailored DG9-conjugated version of the “block” cocktail (five DG9-PMOs, 5 μg/DG9-PMO, 25 μg total dose), and assessed a week later (FIG. 4a). Significant exons 45-55 skipping was observed compared to the saline control (p<0.001) (FIG. 4b). Western blot revealed dystrophin restoration in DG9-PMO-treated muscles at 0.76% of wild-type levels on average, significantly higher than that in saline-treated muscles at 0.46% (p<0.05) (FIG. 4c). As the same mouse received DG9-PMO in one leg and saline in the contralateral leg, protein from a non-treated mouse was included to account for possible leakage between legs. Non-treated tibialis anterior muscles had 0.12% dystrophin of wild-type levels, which was lower than the level in the saline control. A few scattered dystrophin-positive fibers in DG9-PMO-treated muscles were observed, and very few to none in the saline controls, confirming dystrophin restoration (FIG. 4d).

### Discussion

A minimized exons 45-55 skipping cocktail that induces dystrophin restoration in immortalized patient cells and dystrophic hDMDdel52;mdx mice was developed. For exon 52-deleted DMD transcripts, the number of PMOs used for exons 45-55 skipping was reduced from 11 in the “all” to 5 in the “block” cocktail (FIG. 1a), more than a 50% decrease in PMO content. While most minimized cocktails significantly skipped exons 45-55 in KM571 (Δex52) and 6594 (Δex48-50) myotubes, apparently all remaining exons have to be targeted in 6311 myotubes (Δex45-52). The “block” cocktail restored dystrophin production to ˜3% of wild-type levels, near the amount seen with the “all” cocktail (FIG. 1e).

The efficacy of conjugating DG9 to PMOs in single- and multi-exon skipping applications was demonstrated. Treatment with DG9-PMO resulted in higher single-axon skipping and dystrophin restoration levels in vivo compared to unconjugated PMO (FIGS. 2b-d), similar to what has been extensively observed for other cell-penetrating peptides (20, 26). Repeated treatment resulted in greater dystrophin restoration in all examined tissues especially the heart, from 2.5% to 7.7% of wild-type levels (FIGS. 2c,3d).

The advantage of DG9 is in its potentially better toxicity profile compared to other peptides. Peptide-conjugated PMOs have induced dose-dependent toxic effects in pre-clinical studies, including lethargy, weight loss, and kidney damage (20). This is linked to the membrane-disruptive properties of cell-penetrating peptides, which are largely influenced by their amino acid compositions (20, 29). Certain L-arginine residues in DG9 were converted to D-arginina, as this improves the viability of peptide-conjugated PMO-treated cells in vitro (30). DG9 also does not contain any 6-aminohexanoic acid residues, which have been associated with increased toxicity (30). Compared to DG9, previous peptides mostly used L-arginine and contained multiple 6-aminohaxanoic acid residues, but were of similar length and overall arginina content (20). Even though the above modifications decrease the antisense activity of peptide-conjugated PMOs, it comes with the benefit of increased safety. A balance must be struck between efficacy and safety for peptide-conjugated PMOs, as too frequent or too high doses increase toxicity (20, 29). No evidence of toxicity caused by DG9-PMO on the liver and kidney (FIG. 8) was found.

On the topic of efficacy, even though high exon 51 skipping was induced by our DG9-PMO, the dystrophin restoration observed were generally lower than that achieved by other peptide-conjugated PMOs (20, 29). This may be due to a difference in strategy, as the majority of peptide-conjugated PMO studies performed exon 23 skipping (20). Similar discrepancies between exon skipping and dystrophin restoration levels in vivo were observed by Aoki et al. (2010), who skipped exon 51 with PMOs (31), and Aupy et al. (2020), who also skipped exon 51 but with adeno-associated virus-delivered U7 small nuclear RNAs (32). Exon 51-skipped transcripts or proteins may be less stable than their axon 23-skipped counterparts, leading to the reduced dystrophin levels observed. An in vitro study using various internally truncated dystrophin proteins revealed that exon 51-skipped dystrophins were mostly as stable as full-length dystrophin (33), favoring a hypothesis of decreased transcript stability. While previous work has shown that DMD transcript stability has an impact on dystrophin protein production (34, 35), it remains to be determined if this explains the differences in the case of exon 23- and exon 51-skipped transcripts. Another factor would be the animal model used for testing. As humanized dystrophic mice such as hDMDdel52;mdx have only recently been developed, it would be important to characterize the stabilities of the human DMD transcript and protein in these models, to determine if this would have an impact on the efficacy of tested exon skipping therapies.

Despite its relatively reduced activity, functional improvement in mice given repeated doses of DG9-PMO (FIG. 3g,h, FIGS. 7a,b) were observed. Treated hDMDdel52;mdx mice had average dystrophin restoration levels at 2.8-3.9% of wild-type in the skeletal muscles (FIG. 3c). This was accompanied by improvements in fiber size, as seen in the tibialis anterior and diaphragm (FIGS. 7c,e,f). The finding supports the notion that not much dystrophin may be needed to achieve functional benefit in vivo. Indeed, previous studies in mdx mice with non-random X-chromosome inactivation (mdx-Xista¹′) have shown that as little as 3-14% dystrophin of normal levels were sufficient to improve performance in hanging wire and grip strength tests to wild-type levels (36).

In terms of multi-exon skipping, it was promising that the DG9-conjugated version of the minimized “block” cocktail successfully skipped axons 45-55 at 9.5% efficiency and restored dystrophin production in hDMDdal52;mdx mice at nearly 0.8% of wild-type levels, on average (FIGS. 4b,c). The “all” cocktail was previously shown to induce 15% exons 45-55 skipping efficiency upon intramuscular treatment of a different humanized DMD mouse model, at a dose of 1.67 μg/PMO [13]. A higher dose of the “block” cocktail is apparently needed to induce comparable skipping levels to the “all” cocktail in vivo. Because the human and mouse transcript target sequences for the DG9-PMOs in the “block” cocktail are 70-93% identical, the mouse Dnd transcript expressed in hDMDdel52;mdx mice may have sequestered some of the administered DG9-PMOs. Crossing the hDMDdel52 transgene over to a mouse Dmd-null background [37] would eliminate this possibility, and may yield more representative results of efficacy.

### Methods

Cell culture. All immortalized human muscle cells were kindly provided by the MRC Center for Neuromuscular Diseases Biobank (NHS Research Ethics Committee reference 06/Q0406/33, HTA license number 12198), through Dr. Francesco Muntoni. The following immortalized patient-derived myoblast cell lines were used: KM155 (healthy), KM571 (DMD Δex52), 6594 (DMD Δex48-50), and 6311 (DMD Δex45-52). Information on these cell lines are summarized in Table 2. Myoblasts were grown in DMEM/F12 medium (with HEPES; Gibco) containing 20% fetal bovine serum (Sigma), 1 vial of skeletal muscle growth supplement mix (Promocell), 50 U/mL penicillin, and 50 μg/mL streptomycin. Myoblasts were then seeded into collagen type 1-coated 12-well plates, at a density of 0.53×10⁵ cells/cm². Once 90% confluent, myoblasts were differentiated into myotubes by replacing the growth medium with differentiation medium (DMEM/F12 containing 2% horse serum [GE Healthcare], 1×ITS solution [Sigma], 50 U/mL penicillin, and 50 μg/mL streptomycin). All cells were incubated at 37° C., 5% CO₂.

PMO transfection. The PMOs used are summarized in Table 1 and were derived from cocktail set no. 3 in our previous publication [1]. Prior to transfection, PMOs (Gene Tools) were heated at 65° C. for 15 min to remove aggregates. PMOs were then transfected into muscle fibers at 3 days post-differentiation using 6 μM Endoporter reagent (Gene Tools) in differentiation medium. Each PMO in a cocktail was transfected at a final 5 μM concentration for all experiments. Cells were incubated in PMOs for 2 days, after which they were harvested for RNA and protein (FIG. 1a). Random control 25-N(Gene Tools) was used for mock treatment. For non-treated samples, transfection was done as described with Endoporter, only without any PMO.

Animal treatments. Only male hDMDdel52;mdx mice (C57BL/6J background) (2, 3) heterozygous for the hDMDdel52 transgene were used. For single-exon skipping studies, DG9 (sequence N-YArVRRrGPRGYArVRRrGPRr-C: uppercase: L-amino acids, lowercase: D-amino acids) was conjugated to the 3′ end of Ex51_Ac0 (Table 1), a human DMD exon 51-skipping PMO we previously developed [1, 4]. We performed two experiments: single-dose and repeated-dose treatment. For single-dose treatment, 3-month-old hDMDdel52;mdx mice were retro-orbitally injected with either phosphate-buffered saline (PBS), PMO (50 mg/kg, unconjugated Ex51_Ac0), or DG9-PMO (64 mg/kg, equimolar to 50 mg/kg PMO) and then euthanized a week later for tissue collection. For repeated-dose treatment, 2-month-old hDMDdel52;mdx mice were given three retro-orbital injections of either PBS or DG9-PMO (30 mg/kg) once a week for 3 weeks. Body weights were recorded throughout the course of treatment, and mice were subjected to muscle function tests before the first injection and 2 weeks after the last injection as described in Functional testing. Mice were euthanized after the post-function tests for tissue collection. Upon dissection, tissues were mounted in tragacanth gum on corks and snap-frozen in liquid nitrogen-cooled isopentane.

For multi-exon skipping, DG9 was conjugated to each PMO of the minimized “block” exons 45-55 skipping cocktail (FIG. 1a). This DG9-PMO cocktail (5 μg per DG9-PMO, total dose of 25 μg) was then administered intramuscularly into the tibialis anterior of 5- to 6-month-old hDMDdel52;mdx mice: the contralateral leg was injected with PBS. Mice were euthanized a week later for tissue collection as described above. All injections, retro-orbital and intramuscular, were conducted under isoflurane anesthesia. Tissues from age-matched wild-type male C57BL16J mice were collected as controls.

Functional testing. Forelimb and total limb grip tests were conducted by blinded personnel according to TREAT-NMD SOP DMD_M.2.2.001 using the Chatillon DFE II grip strength meter (Columbus Instruments). The average of the three most consistent readings was used per mouse, and results were normalized to body weight. The rotarod test was performed using the AccuRotor 4-channel rotarod (Omnitech Electronics, Inc.). Mice were first placed on a rod rotating at a steady speed of 5 rpm. Once all mice were in place, rotation was accelerated from 5 to 45 rpm over a span of 300 s [5]. Fall times were automatically recorded by the software. Three trials were conducted, spaced 15 min apart, and the average or peak fall time from these trials was used for analysis. The run-to-exhaustion testwas performed using the Exer 3/6 animal treadmill (Columbus Instruments), according to TREAT-NMD SOP DMD_M.2.1.003. Mice were run on the treadmill with the following program: 5 m/min for 5 min, and then speed is increased by 1 m/min every minute until exhaustion. Exhaustion is considered as the point when the mouse does not get back on the treadmill within 10 s following repeated, gentle nudges. The maximum test duration was set at 15 min. All tests were performed at baseline (prior to receiving treatment) and at 2 weeks after receiving final treatment. Age-matched wild-type male C57BL/6J mice were used as controls.

RT-PCR and exon skipping evaluation. Total RNA was extracted from cells and 20-μm tissue sections using Trizol (Invitrogen), following the manufacturer's instructions.

For exons 45-55 skipping analysis, the SuperScript™ III One-Step RT-PCR system with Platinum™ Taq (Invitrogen) was used. Briefly, 200 ng of total RNA was used as template in a 25-μL solution containing 1× reaction mix, 0.2 μM each of forward and reverse primers for DMD or GAPDH/Gapdh (Table 3), and 1 μL of SuperScript III RT/Platinum Taq. The reaction was run under the following conditions: 1) 50° C., 5 min, 2) 94° C., 2 min, 3) 35 cycles of 94° C., 15 s; 60° C., 30 s; 680° C., 33-118 s, 4) 680° C., 5 min, 5) 4° C., hold.

For exon 51 skipping analysis, cDNA was synthesized from 750-1000 ng of total RNA using SuperScrip™ IV Reverse Transcriptase (Invitrogen) with 2.5 μM random hexamers (Invitrogen) in a 20-μL reaction following manufacturer's instructions. From this, 8 μL of cDNA was used for PCR with 1× GoTaq® Green Master Mix (Promega) and 0.3 μM each of forward and reverse primers for DMD or Gapdh (Table 3) in a 25-μL reaction. The reaction was run as follows: 1) 95° C., 2 min, 2) 40 cycles of 95° C., 30 s; 60° C., 30 s; 72° C., 35 s, 3) 72° C., 5 min, and 4) 4° C., hold. All PCR products (exons 45-55 or exon 51 skipping) were run in 1.5% agarose gels in 1× tris-borate-EDTA buffer, and band intensities were quantified by Image J (NIH). The % of successful exon skipping was calculated using the following formula: (intensity of desired skipped band/total intensity of unskipped, intermediate, and desired skipped bands)×100.

Western blot. Total protein was extracted from cells using RIPA buffer (Sigma) supplemented with cOmplete, Mini, EDTA-free protease inhibitor cocktail (Roche), according to our previously published protocol (6). On the other hand, total protein was extracted from 20-μm tissue sections using a high-SDS lysis buffer containing 10% SDS, 70 mM Tris-HCl (pH 6.7), 5 mM EDTA (pH 8.0), 5% β-mercaptoethanol, and cOmplete protease inhibitor cocktail in water, also according to a previous protocol (7). Proteins were quantified using the Pierce™ BCA kit (Thermo Scientific) or the Pierce™ Coomassie (Bradford) kit (Thermo Fisher), respectively.

In preparation for Western blot, proteins were mixed with NuPAGE™ LDS Sample Buffer (Invitrogen; 1× final concentration) and NuPAGE™ Sample Reducing Agent (Invitrogen; 1× final concentration), then heated at 70° C. for 10 min. SDS-PAGE was performed using pre-cast NuPAGE™ 3-8% Tris-Acetate Midi gels (Invitrogen), run at 150 V for 75 min. Proteins were then transferred onto a PVDF membrane (Millipore) using a semi-dry blotting system at 20 V for 70 min. Membranes were blocked overnight in 2% ECL Prime Blocking Agent (GE Healthcare) while shaking at 4° C.; the post-transfer gel was stained with PageBlue Protein Staining solution (Thermo Scientific) for 1 hr at room temperature to detect myosin heavy chain bands. After blocking, membranes were cut and incubated in one of the following primary antibodies for 1 hr at room temperature: 1:200 NCL-DYS1 (Leica), 1:2,500 anti-dystrophin C-terminal (Abcam, ab15277), 1:100 MANEX45A (Developmental Studies Hybridoma Bank; DSHB), (8) 1:100 MANEX4850E (MDA Monoclonal Antibody Resource, Wolfson Centre for Inherited Neuromuscular Disease) (8), or 1:4,000 anti-desmin (Abcam, ab8592) in blocking agent. The membranes were then washed thrice for 10 min each with PBS containing 0.05% Tween 20 (PBST), before incubating in either anti-mouse IgG2a, anti-mouse IgG1, or anti-rabbit IgG (H+L) horseradish peroxidase-conjugated secondary antibodies (Invitrogen) as appropriate, all 1:10,000 in PBST. Membranes were then similarly washed in PBST, and detected with ECL Select Detection Reagent (GE Healthcare). DYS1 band intensities were quantified using Image Lab™ software, v.6.0.1 (Bio-Rad), and dystrophin levels were expressed relative to the intensity of the least concentrated wild-type sample in a series.

Dystrophin immunofluorescence. Frozen muscle and heart samples were sectioned at 7-μm thickness and placed on poly-L-lysine-coated slides. After thawing at room temperature for 30 min, sections were blocked for 2 hr in PBS with 10% goat serum and 0.1% Triton X-100 at room temperature. Sections were then incubated with 1:50 NCL-DYS1 in the blocking agent overnight at 4° C. The following day, sections were washed thrice with PBS for 5 min each and subsequently incubated with Alexa 488-conjugated goat anti-mouse IgG2a secondary antibody (Life Technologies) for 30 min at room temperature. Sections were washed again with PBS, and mounted with Vectashield HardSet Antifade Mounting Medium with DAPI (Vector Laboratories). Samples were visualized for dystrophin and DAPI using a Zeiss LSM 710 confocal microscope at 200× magnification, by personnel blinded to the treatment condition.

Histology. Frozen muscles were sectioned at 7-μm and placed on poly-L-lysine-coated slides. After thawing at room temperature for 30 min, slides were stained with Mayer's hematoxylin (Electron Microscopy Sciences) for 15 min, washed with running tap water for 15 min, and then stained with eosin Y (Electron Microscopy Sciences) for 10 min. Sections were then dehydrated with an ethanol series (70%-90%-99%), cleared with a xylene substitute, and mounted with Permount™ (Fisher Scientific). Blinded personnel visualized the samples under brightfield using the Optika B-290 TB microscope at 200× magnification, taking three randomly chosen fields of view per sample. CNF percentage was calculated by (#CNFs/total #fibers)×100, with fibers counted manually using Image J. The average CNF percentage from all fields of view was taken per sample. Minimal Feret's diameters were quantified by blinded personnel in two steps. First, images were semi-automatically measured using an in-house developed Image J macro based on Open-CSAM (9). As Open-CSAM was initially developed for immunofluorescence images, we had to extensively modify it for compatibility with hematoxylin and eosin-stained images, which required the use of the Colour Deconvolution 2 plugin [10, 11]. Second, images that passed semi-automatic measurement were manually curated to correct fiber boundaries. Individual fiber measurements across samples were considered for analysis. For both CNF and minimal Feret's diameter quantification, fibers that touched the edges of an image were not considered.

Statistical Analysis. All statistical tests were performed using Prism v.9.0.1 (GraphPad Software). Unpaired two-tailed t-test, or one-way ANOVA with post-hoc Tukey's or Dunnett's multiple comparisons test were conducted as appropriate. P-values<0.05 were considered statistically significant.

TABLE 1
List of PMO sequences used in this study.

| ID* | Sequence, 5′ to 3′ |
|---|---|
| Ex45_Ac9 | GACAACAGTTTGCCGCTGCCCAATGCCATC |
| Ex46_Ac93 | AGTTGCTGCTCTTTTCCAGGTTCAAGTGGG |
| Ex47_Ac13 | GTTTGAGAATTCCCTGGCGCAGGGGCAACT |
| Ex48_Ac7 | CAATTTCTCCTTGTTTCTCAGGTAAAGCTC |
| Ex48_Ac78 | CAGATGATTTAACTGCTCTTCAAGGTCTTC |
| Ex49_Ac17 | ATCTCTTCCACATCCGGTTGTTTAGCTTGA |
| Ex50_Ac19 | GTAAACGGTTTACCGCCTTCCACTCAGAGC |
| Ex51_Ac0 | GTGTCACCAGAGTAACAGTCTGAGTAGGAG |
| Ex52_Ac24 | GGTAATGAGTTCTTCCAACTGGGGACGCCT |
| Ex53_Ac26 | CCTCCGGTTCTGAAGGTGTTCTTGTACTTC |
| Ex54_Ac42 | GAGAAGTTTCAGGGCCAAGTCATTTGCCAC |
| Ex55_Ac0 | TCTTCCAAAGCAGCCTCTCGCTCACTCACC |

*represented as: exon target_distance from target exon acceptor site

TABLE 2
Information on the immortalized cell lines used in this study.

| | | | Patient | |
|---|---|---|---|---|
| Cell line | DMD mutation | Source tissue | sex | Patient age |
| KM155 | n/a (healthy) | Right semitendinosus | male | 25 years |
| KM571 | Δex52 | Fascia-lata | male | 10 years |
| 6594 | Δex48-50 | Left quadriceps | male | 20 months |
| 6311 | Δex45-52 | Left quadriceps | male | 23 months |

TABLE 3
List of primers and sequences
used in this study.

| | | Product size/s, |
|---|---|---|
| Target | Sequence, 5′ to 3′ | bp |
| hDMD | F: GACAAGGGCGATTTG | 2088 (wild-type), |
| (ex45-55) | ACAG (ex43/44) | 1970 (Δex52), |
| | R: TCCGAAGTTCACTCC | 1691 (Δex48-50), |
| | ACTTG (ex56) | 866 (Δex45-52), |
| | | 309 (Δex45-55) |
| hDMD | F: CAGCCAGTGAAGAGG | 453 (Δex52), |
| (ex51) | AAGTTAG (ex49/50) | |
| | R: CCAGCCATTGTGTTG | 220 (Δex51-52) |
| | AATCC (ex53) | |
| hGAPDH | F: TCCCTGAGCTGAACG | 218 |
| | GGAAG | |
| | R: GGAGGAGTGGGTGTC | |
| | GCTGT | |
| mGapdh | F: CAACTTTGGCATTGT | 381 |
| | GGAAGG | |
| | R: GAAGAGTGGGAGTTG | |
| | CTGTT | |

TABLE 4
List of PMO sequences useful
for exon 44 skipping

| Name | Oligo sequence |
|---|---|
| hEx44_Ac7 | AAAACGCCGCCATTTCTCAACAGATCTGTC |
| hEx44_Ac2 | GCCGCCATTTCTCAACAGATCTGTCAAATC |
| hEx44_Ac14 | CATAATGAAAACGCCGCCATTTCTCAACAG |
| hEx44_Ac0 | CGCCATTTCTCAACAGATCTGTCAAATCGC |
| hEx44_Ac18 | ATATCATAATGAAAACGCCGCCATTTCTCA |
| hEx44_Ac56 | ACTGTTCAGCTTCTGTTAGCCACTGATTAA |
| hEx44_Ac118 | CTTAAGATACCATTTGTATTTAGCATGTTC |
| hEx44_Ac85 | ATTCTCAGGAATTTGTGTCTTTCTGAGAAA |

### Example 2: Peptide-Conjugated PMO for Treatment of Spinal Muscular Atrophy

Spinal muscular atrophy (SMA) is the most common genetic cause of infant mortality resulting from mutations in the survival motor neuron 1 (SMN1) gene. Humans have an SMN1 paralog SMN2. However, SMN2 cannot rescue the SMA phenotype because 90% of exon 7 in SMN2 mRNA is spliced out due to a single C-to-T nucleotide transition. The currently approved antisense oligonucleotide (AO) nusinersen (brand name Spinraza) targets SMN2 and induces SMN expression by promoting exon 7 inclusion. However, delivering AOs to both the central nervous system (CNS) and bodywide tissues is difficult. Although current approaches have been restricted to using highly invasive intrathecal injections and treats mainly motor neurons, recent studies indicate that SMA is a multi-organ disorder, calling for a body-wide treatment approach. To bypass intrathecal injections a novel cell-penetrating peptide called DG9 that increases the uptake of AO significantly following a single subcutaneous administration. It was demonstrated that DG9-conjugated AO (DG9-PMO) extends the lifespan of SMA mice and improves breathing dysfunction, which is the most common cause of mortality in SMA. DG9-PMO treatment leads to a significant increase in full-length SMN2expression and SMN protein levels in both peripheral and central nervous system tissues. Additionally, the treatment also improved the pathology of skeletal muscles and neuromuscular junctions. Lastly, we show that DG9-PMO penetrates a fully-developed blood brain barrier, and can distribute in the CNS following a systemic injection.

### Results

### Subcutaneous Administration of DG9-PMO Enhances Survival and Increases SMN2 Expression in Severe SMA Mice in a Dose-Dependent Manner.

To examine whether DG9 peptide can increase the efficacy of PMO, DGS peptide was conjugated to an 18-mer PMO with a sequence identical to nusinersen. The DG9-conjugated PMO and unconjugated-PMO and 2′-O-methoxyethyl-RNA (MOE) were injected into SMA model mice. We used the Taiwanese SMA mouse model (Smn^{-/-} SMN2^{Tg/-}) that exhibits a severe phenotype and has a median survival of 8 days (d) [77,78].

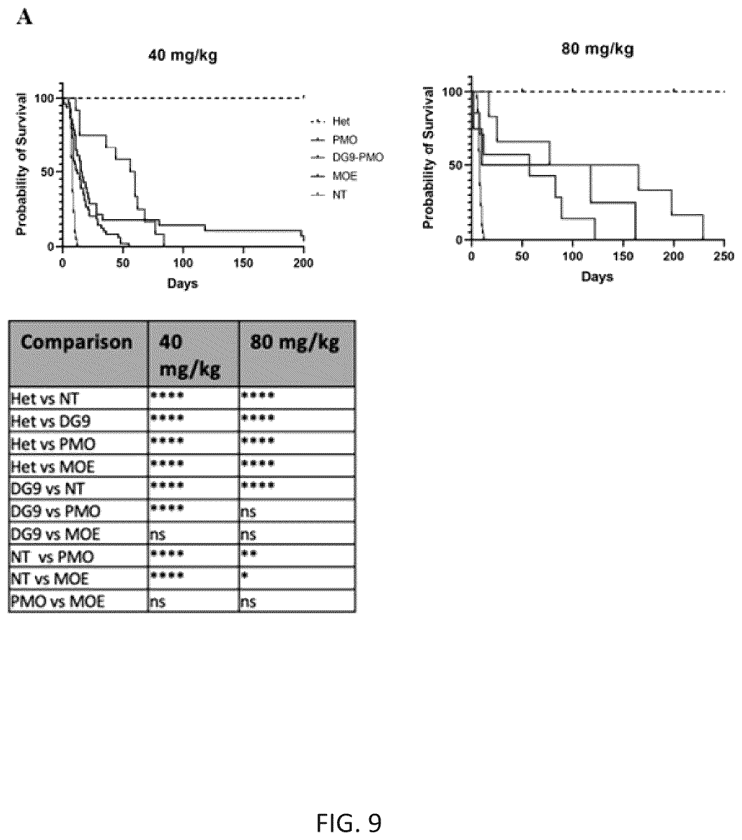

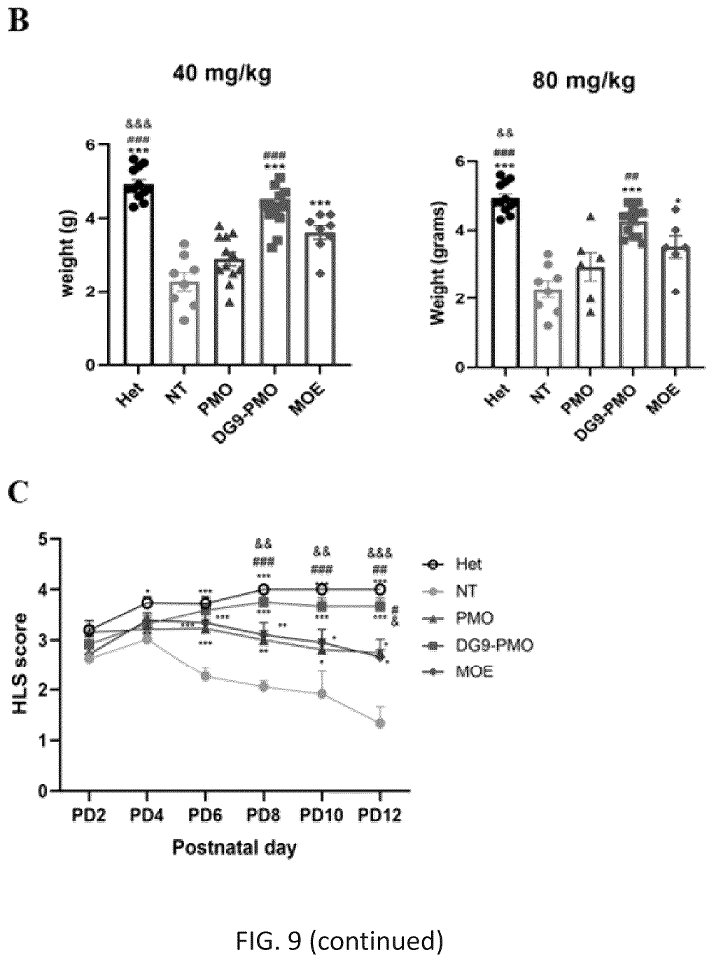

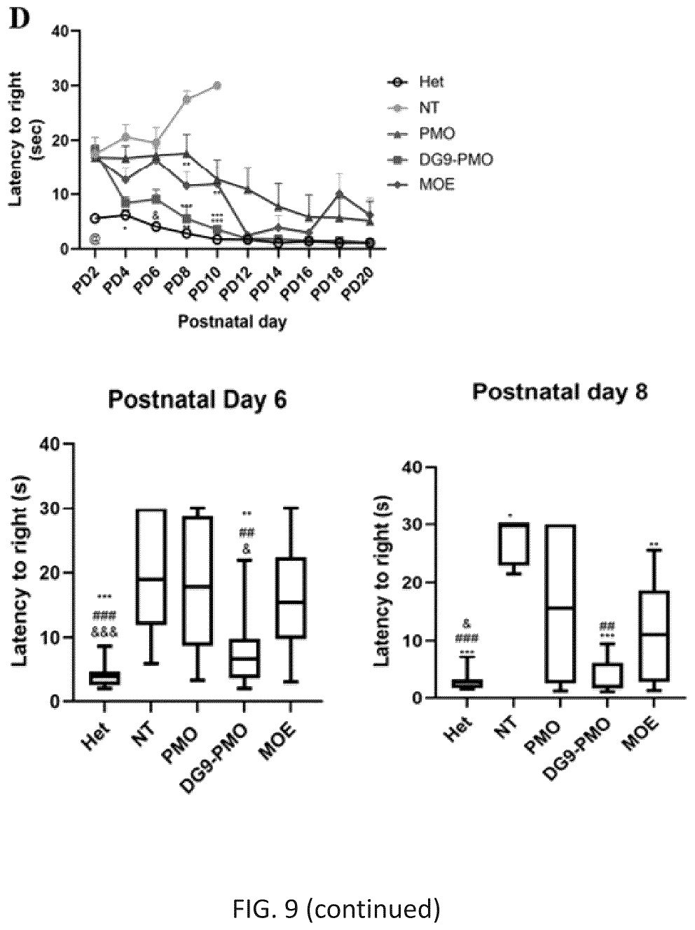

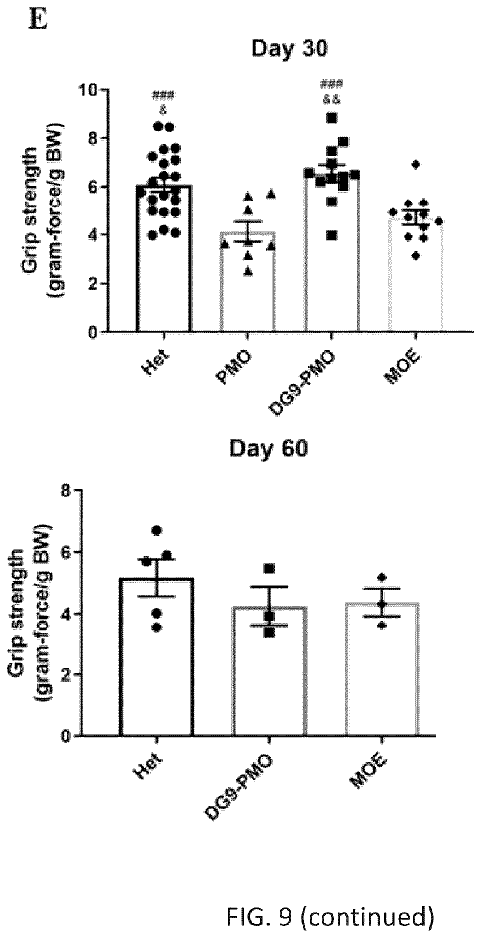

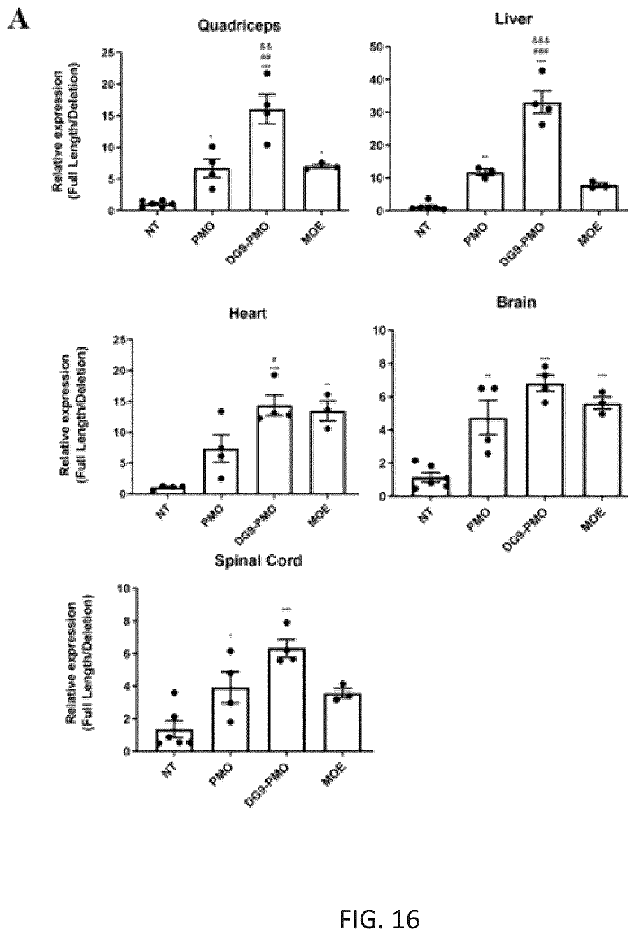

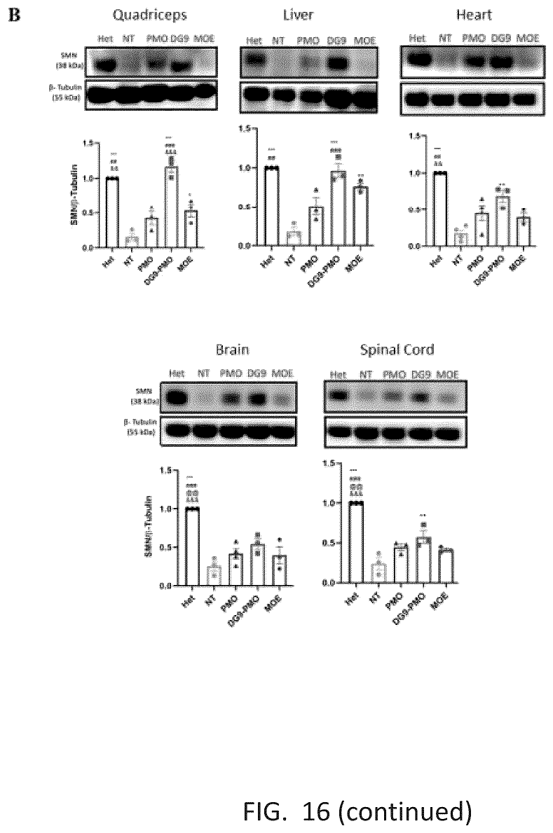

Mice were subcutanteously injected with the AOs or saline (NT) administered at postnatal day 0 (PD0) at two different doses-40 mg/kg or 80 mg/kg. The survival was recorded until a humane endpoint was reached. For the 40 mg/kg doses, the median survival was 12 d (unconjugated-PMO), 15.5 d (MOE), and increased to 58 d for DG9-PMO treatment (FIG. 9a). The median survival incremented to 57 d (unconjugated-PMO), 121 d (DG9-PMO) and 64 d (MOE) for the higher dose of 80 mg/kg (FIG. 9a). This result indicates that DG9-PMO can increase life-span in the SMA mice more effectively than the unmodified PMO. For both doses, DG9-PMO treated mice were significantly heavier than the NT, unconjugated-PMO treated littermates at postnatal day 7 (PD7), with no significant difference in weights when compared to the age-matched heterozygous littermates (Hets) (Smn^{+/-}, SMN2^{Tg/-}) that were used as healthy controls exhibit normal wild-type (AT) like-characteristics (FIG. 9b). The DG9-PMO treated mice looked very similar to the Hets, while the unconjugated PMO-treated neonates looked exhibited a weak phenotype-similar to the NT mice (FIG. 16)

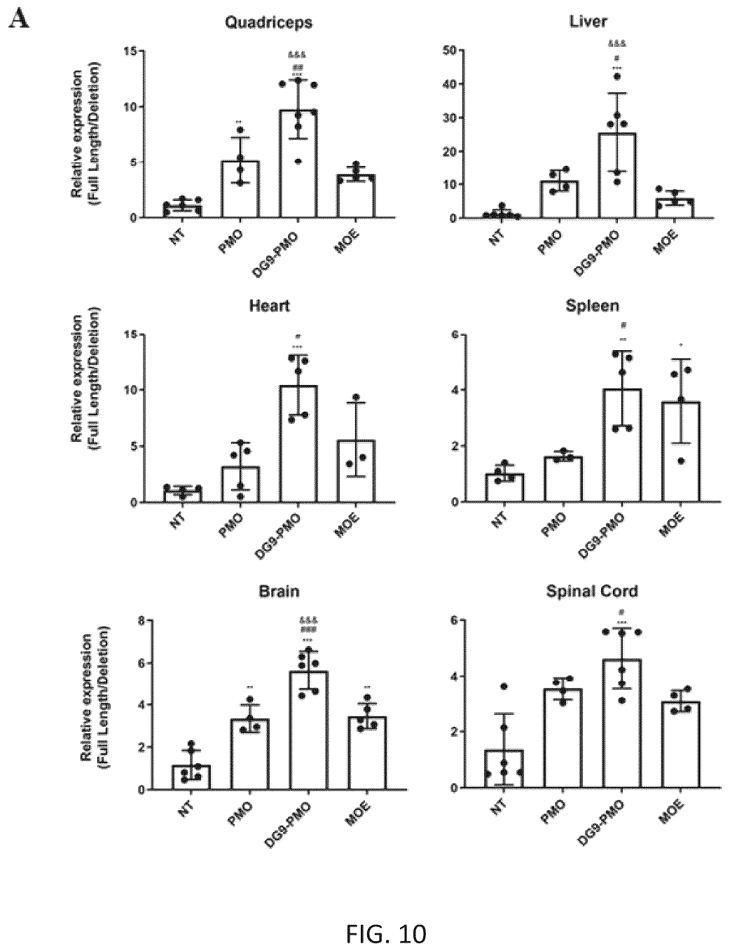

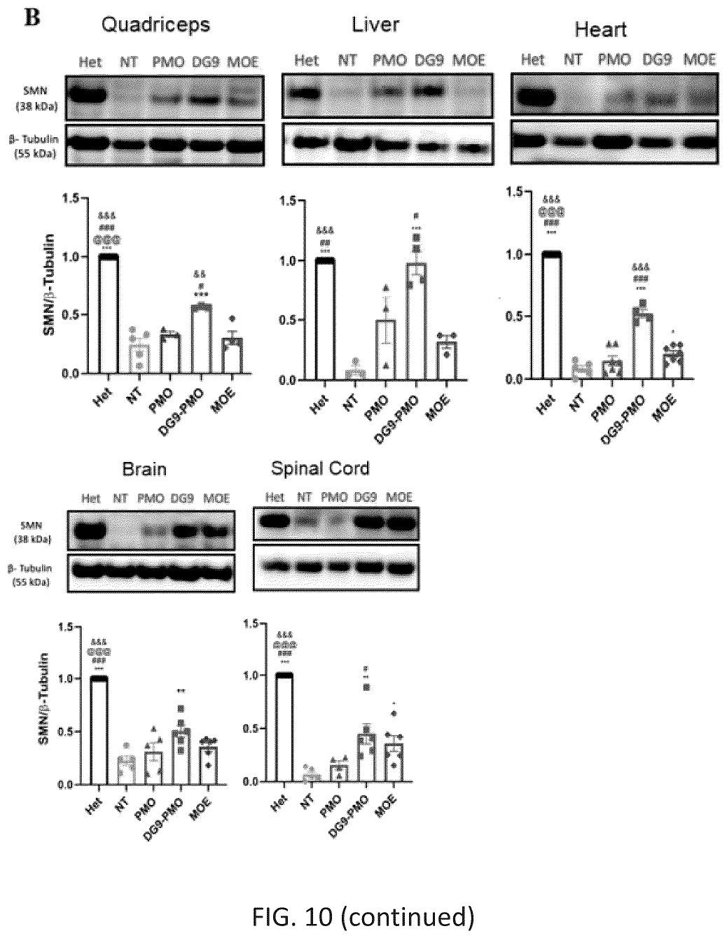

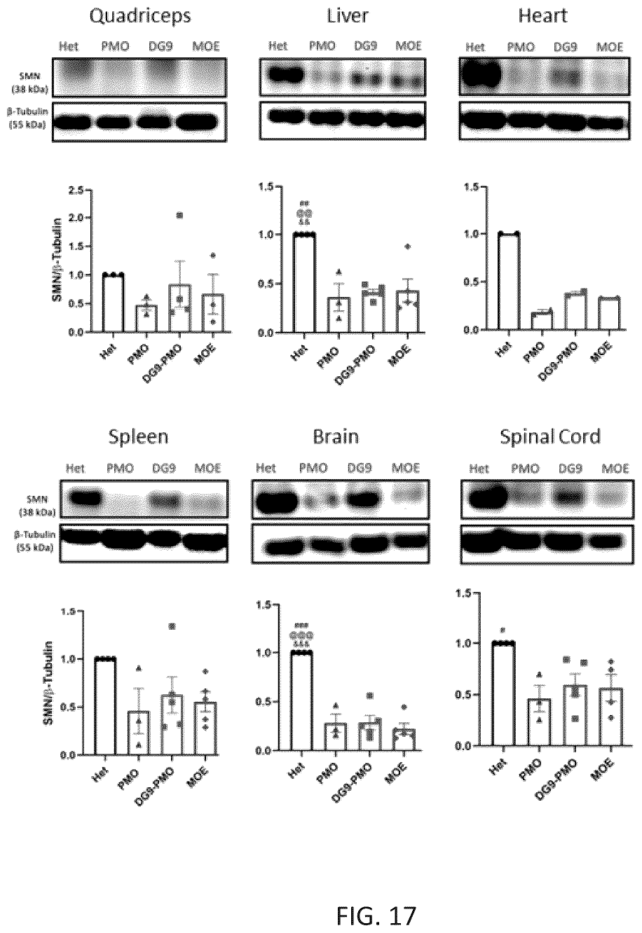

Next, the FL-SMN2 expression was evaluated relative to Δ7 SMN2 transcripts using real-time quantitative PCR (RT-qPCR) at PD7. In both doses, DG9-PMO treatment led to a 4-to-30-fold higher FL-SMN2 expression than NT control in both peripheral and CNS tissues (FIG. 10a). It also led to a ˜5-fold increase in FL-SMN2 expression when compared to unconjugated-PMO and MOE treatments in majority of the tissues (FIG. 10a, FIG. 16a). These data demonstrate that DG9-PMO can induce FL-SMN2 expression more efficiently than the unmodified PMO. These findings were validated at the protein level using western blotting where DG9-PMO treatment increased SMN protein levels in both the peripheral and CNS tissues, (FIG. 10b, FIG. 16b). We also found that subcutaneous administration of DG9-PMO at 40 mg/kg led to sustained levels of SMN expression at PD30 (FIG. 17). DG9-PMO and MOE treatments had similar SMN levels in the studied tissues. These results indicate the greater efficacy of DG9-PMO than unconjugated PMOs with a dose-dependent effect.

### Subcutaneous Administration of DG9-PMO Improves Motor Function and Muscle Strength in SMA Mice.

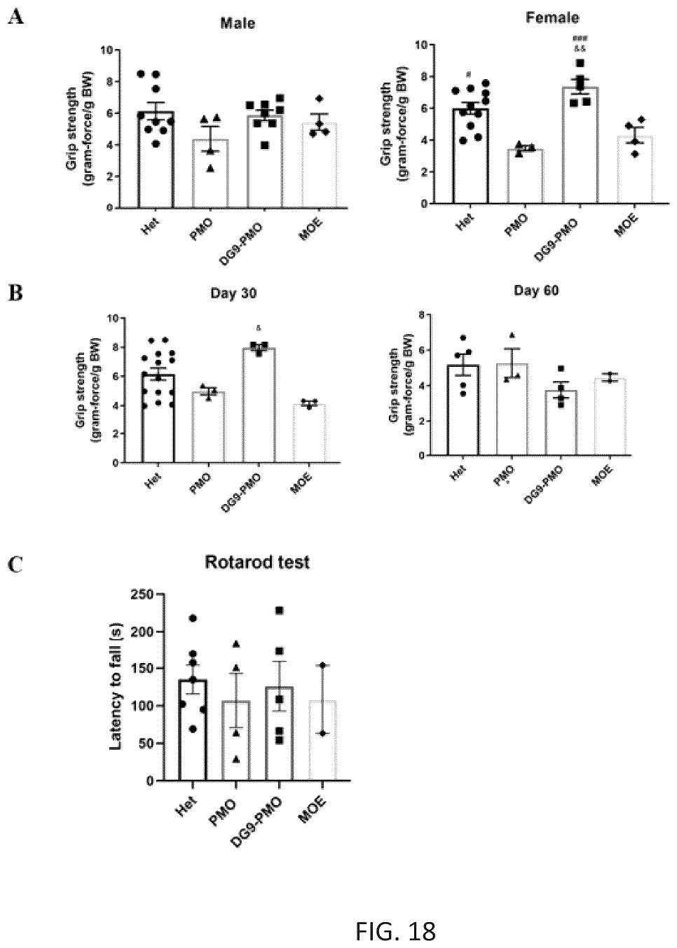

To evaluate the effects of systemic DG9-PMO treatment on muscle strength and the function of motor neurons, several functional tests were used to compare motor function in the treated and NT mice at various time-points. The hindlimb suspension (HLS) assay and the righting-reflex test were performed during the early weeks of life. After PD6, NT mice had a decreasing HLS score as they could not extend their hindlimbs when suspended by the tail (FIG. 9c). These mice also exhibited a reduced latency on the tube. At PD12, DG9-PMO treated neonates exhibited hindlimb strength comparable to the Hets, with a significantly higher score and greater latency on the tube than the unconjugated-PMO and NT mice (FIG. 9c). In the righting-reflex test, DG9-PMO mice took a significantly shorter time to right themselves on their paws between PD6-PD10, suggesting improved muscle strength and coordination (FIG. 9d). The unconjugated-PMO and MOE-treated mice that survived beyond PD12 improved in performance but still took longer than DG9-PMO mice. It should be noted that these results are slightly skewed as only the healthiest unconjugated-PMO, and MOE-treated mice survived to these time points. This improvement was further bolstered by the forelimb grip strength assessment at PD30 in the DG9-PMO-treated mice (FIG. 9e). DG9-PMO-treated mice exhibited a forelimb grip force comparable to the Hets, and significantly higher than the unconjugated-PMO and MOE-treated mice (FIG. 9e). We also evaluated the relationship between the forelimb data and the sex at PD30. Surprisingly, no significant difference between males across the groups was observed but significant differences in the females (FIG. 18a) was observed. At PD60, the performances demonstrated by the Hets, and treated mice were not significantly different (FIG. 9e). Unconjugated-PMO mice did not survive until PD60 and were excluded from this portion of the study. We observed similar results in the 80 mg/kg dose study both at PD30 and PD60 (FIG. 18b). We also performed a rotarod test PD30 for the 40 mg/kg groups to assess their overall muscle coordination and balance. The mice across all groups were variable in their ability to coordinate and balance themselves on the rotating beam, with no significance detected [36](FIG. 18c). Based on conclusive findings from these functional tests, systemic injection of DG9-PMO improves motor function and muscle strength of SMA mice.

### DG9-PMO Treatment Improves Breathing Function in Neonatal SMA Mice

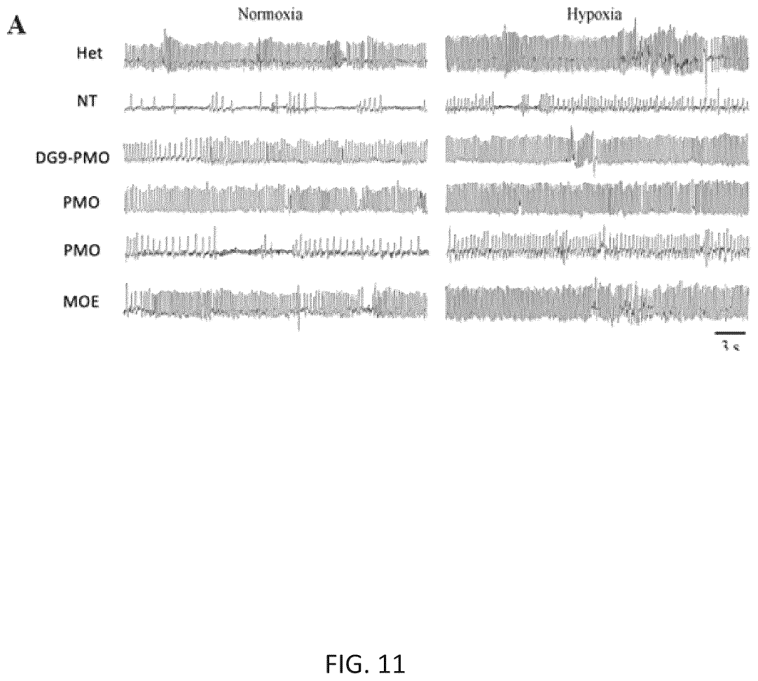

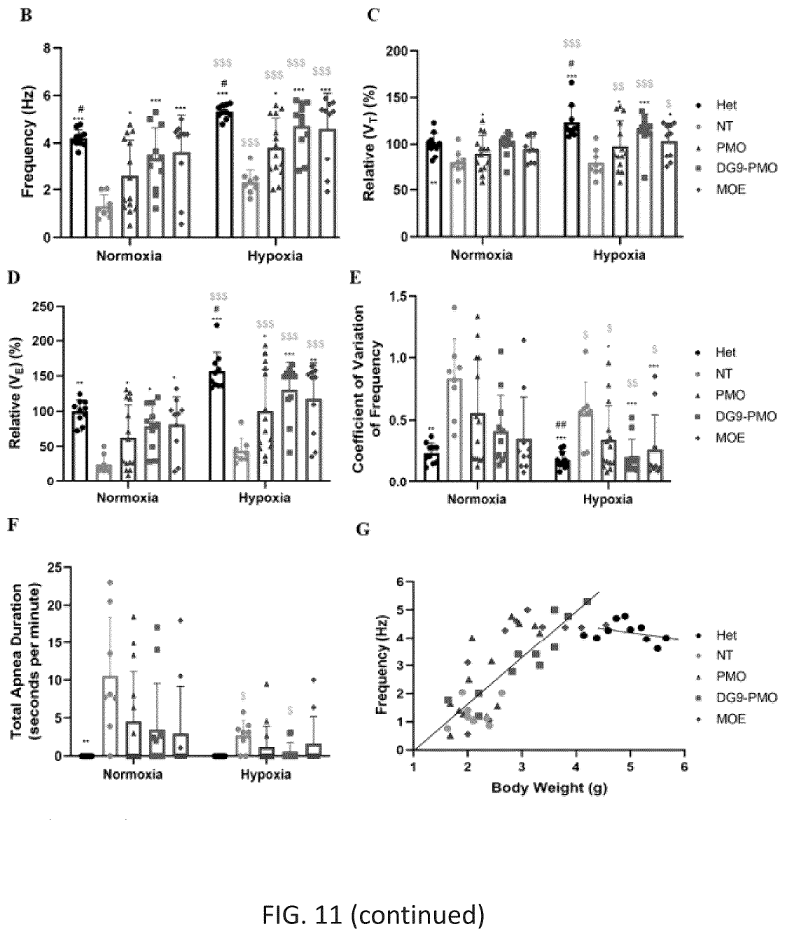

Majority of the SMA patients suffer from compromised breathing functions and rely on an external source of breathing support. To evaluate the effects of DG9-PMO treatment on breathing function, we performed whole-body plethysmography recordings at PD7 under normoxic (21% 02) and hypoxic (11% 02) conditions (FIG. 11a). The Hets did not present a respiratory phenotype under normoxic conditions. The NT mice on the other hand had slow, irregular breathing denoted by a higher coefficient of variation of frequency (CoV, a measure of relative variability), and marked apneas (absence of airflow/pressure changes for a period equivalent or greater than two complete respiratory cycles) (FIG. 11b, f). Half of the unconjugated-PMO mice (n=14) exhibited parameters as seen in NT mice, while the other half were like the Hets and demonstrated an increase in respiratory frequency (f_{R}, number of breaths per minute), minute ventilation (V_{E}), and tidal volume (V_{T}, amount of air flowing in or out of the lungs during each respiratory cycle) when compared to the NT control (FIG. 11b, d). Under the same normoxic conditions, majority of the DG9-PMO (n=11) and MOE (n=10) treated mice did not exhibit any respiratory phenotype and were similar to the Hets (FIG. 11b-f).

When switched to a hypoxic environment, the Hets, DG9-PMO, and MOE-treated mice exhibited an increase in the respiratory parameters—f_{R} V_{E}, and V_{T} relative to normoxia (FIG. 11b-d). Even though hypoxia reduced the number of apneas, breathing was still slow, and irregular as seen by the high CoV in NT mice (FIG. 11e). Most of the unconjugated-PMO neonates exhibited an increase in the respiratory parameters relative to normoxia with no effects on apnea when compared to NT mice, but still had slow, weak, and irregular breathing when compared to the Hets (FIG. 11b-d). The correlation of the severity of respiratory phenotype (decrease in f_{R} or V_{E}) with decrease in body weight was examined by using the Pearson product moment correlation t-test (FIG. 11g). We observed no correlation between f_{R} and body weight in the Hets, but found a strong correlation in the NT and treated mice (grouped as homozygotes). With a lower CoV and a marked improvement in respiratory parameters, DG9-PMO treatment ameliorated the breathing dysfunction seen in SMA mice.

### DG9-PMO Treatment Improves Muscle Pathology and Neuromuscular Junction (NMJ) Characteristics in SMA Mice.

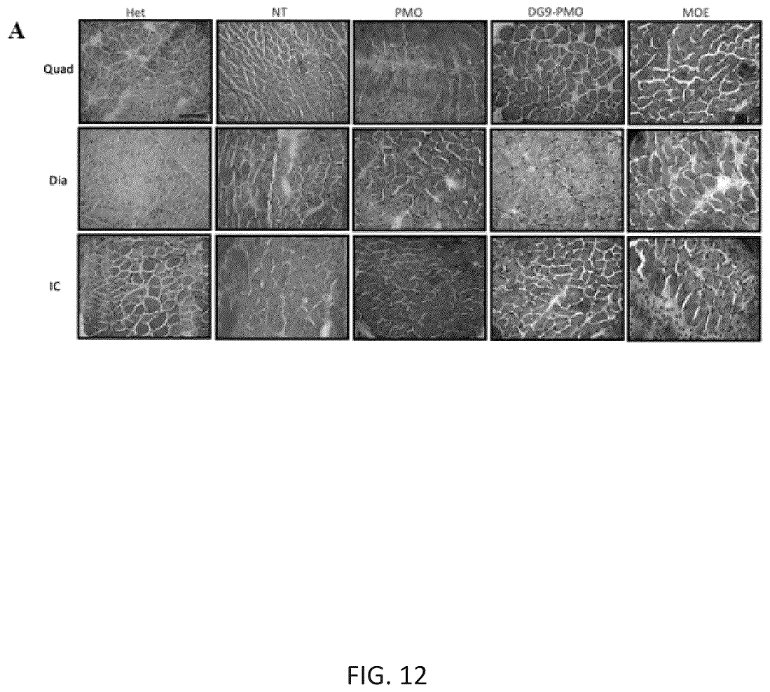

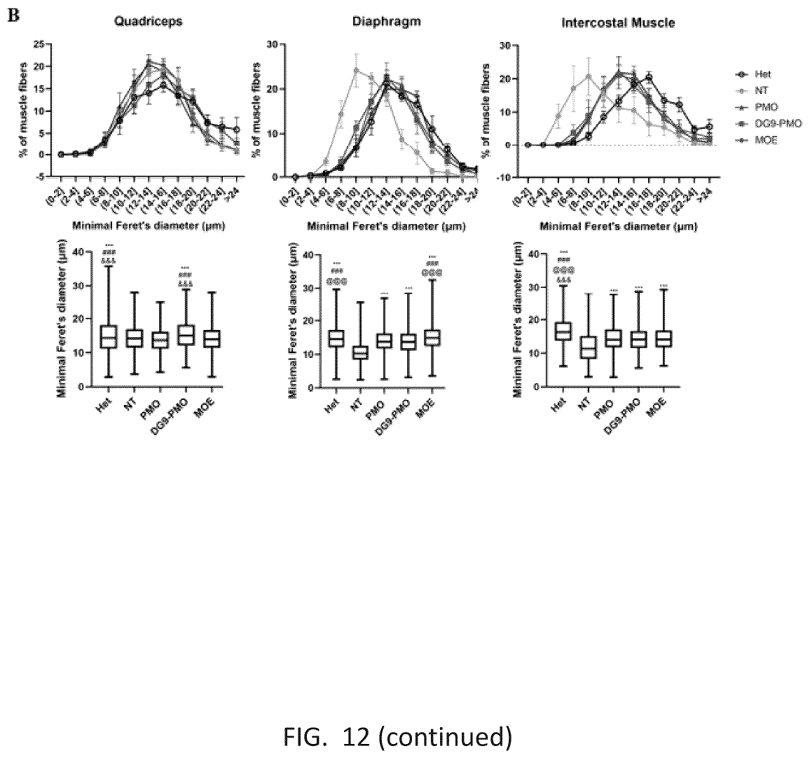

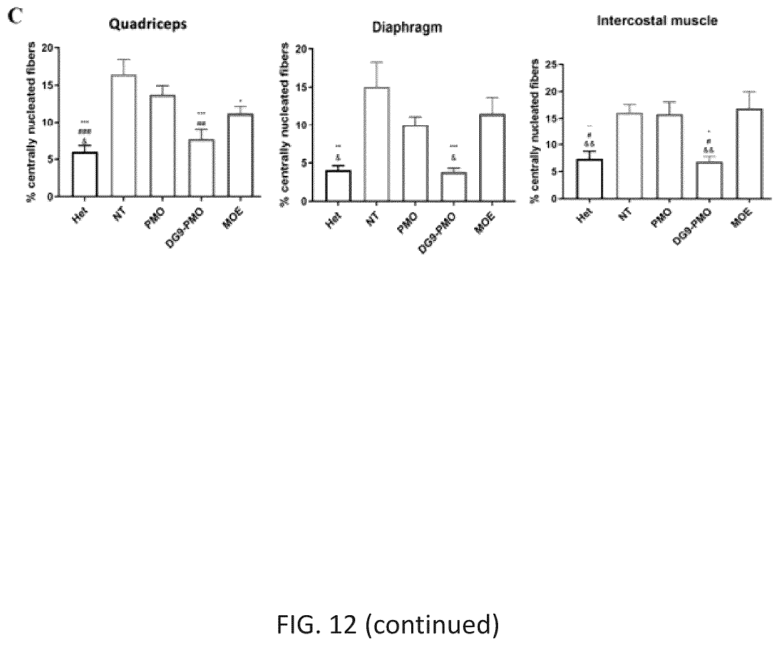

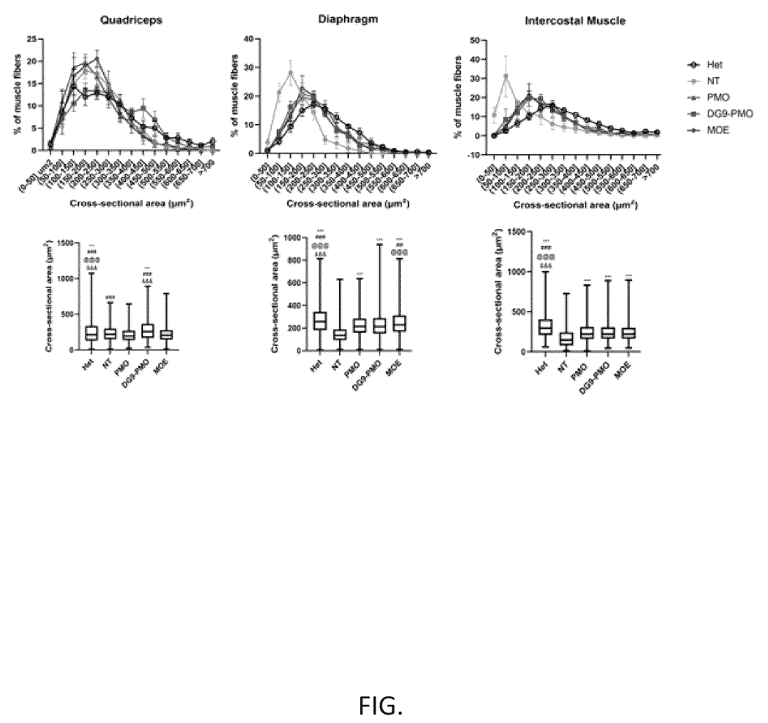

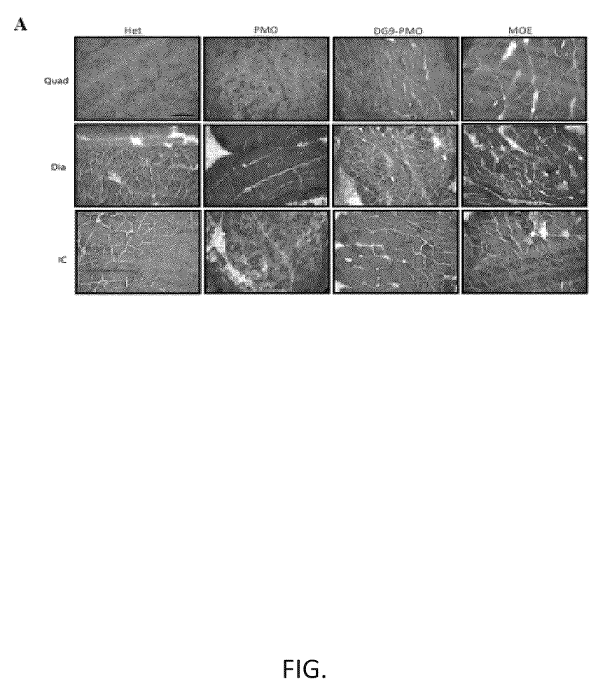

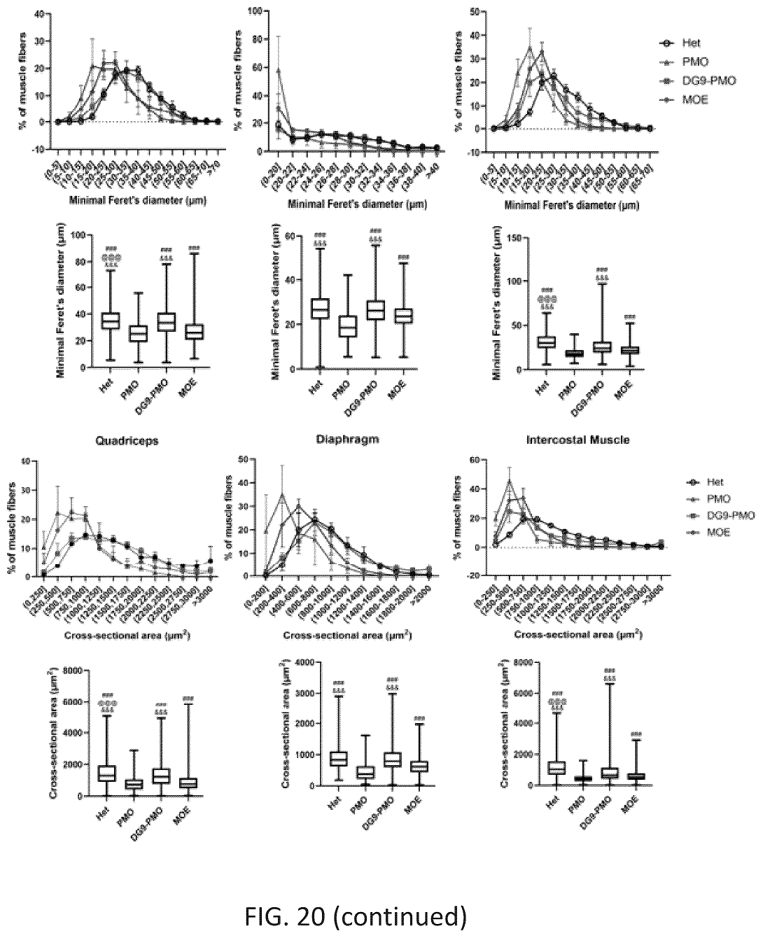

Atrophic musculature is a classical characteristic feature of SMA with diminished skeletal muscle fiber size. To determine the treatment effects on muscle pathology, we assessed the physiology and architecture of the myofibers of two affected muscle groups− the quadriceps and intercostal muscles, as well as the sparingly affected diaphragm at PD7 and PD30 [79,80]. We quantified the cross-sectional area (CSA), the minimal feret's diameter and centrally nucleated fibers for at least 500 myofibers per muscle for each treatment using hematoxylin & eosin (H & E) staining at PD7 and PD30 (FIG. 12a, b, FIGS. 19 and 20).

At PD7, the feret's diameter and CSA of the myofibers were significantly larger in all three muscle groups following DG9-PMO treatment when compared to the NT control (FIG. 12b, FIG. 19). In the quadriceps muscle, there was no significant difference between the Hets and the DG9-PMO treated myofiber size. On the other hand, unconjugated-PMO and MOE quadriceps myofibers were significantly smaller than those of DG9-PMO. In the diaphragm and intercostal muscle, all three treatment groups exhibited a similar myofiber size (FIG. 12b, FIG. 19). In addition, DG9-PMO treatment did lead to a significant decrease in the percent of centrally nucleated fibers, indicative of degenerative myofibers in atrophic myofibers. (FIG. 12c). The effect of DG9-PMO persisted at PD30 (FIG. 20). Unconjugated-PMO and MOE treated mice had significantly smaller myofibers and a higher percent of central nuclei compared to DG9-PMO in all three tissue types (FIG. 20c).

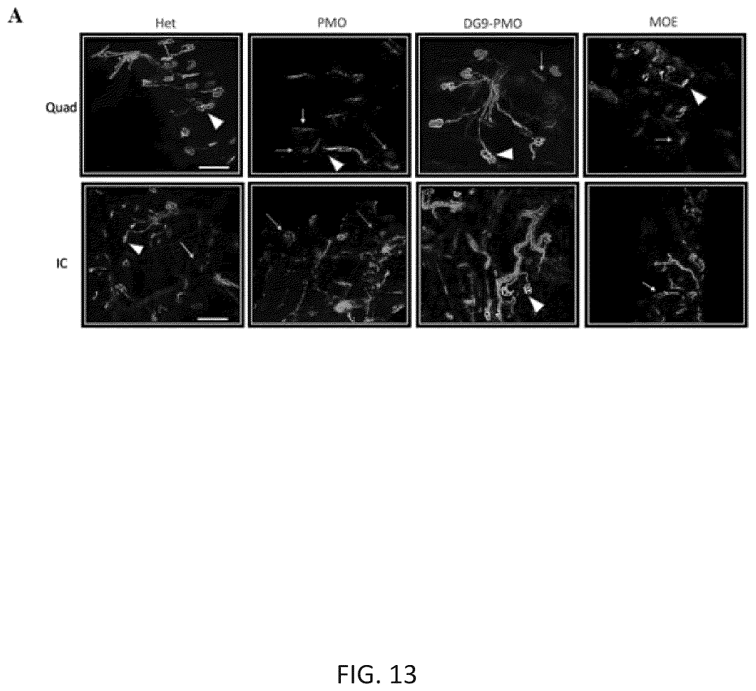

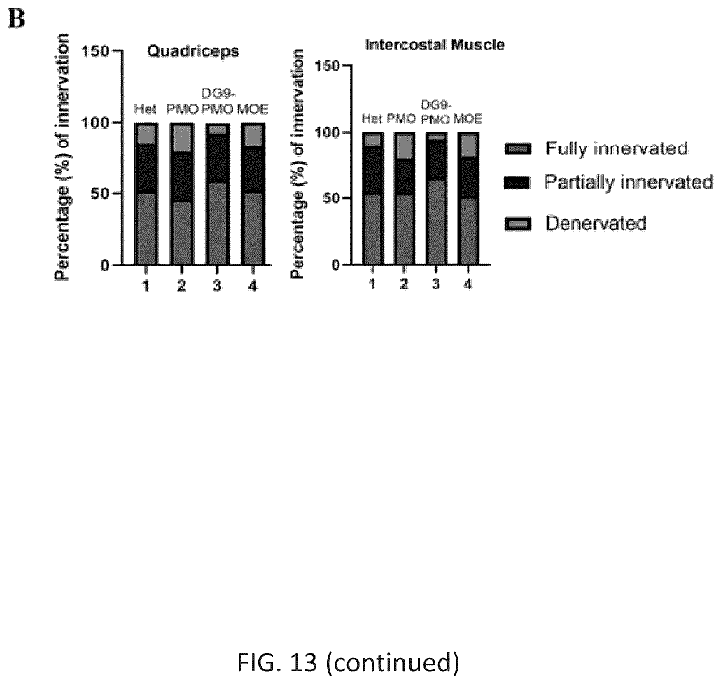

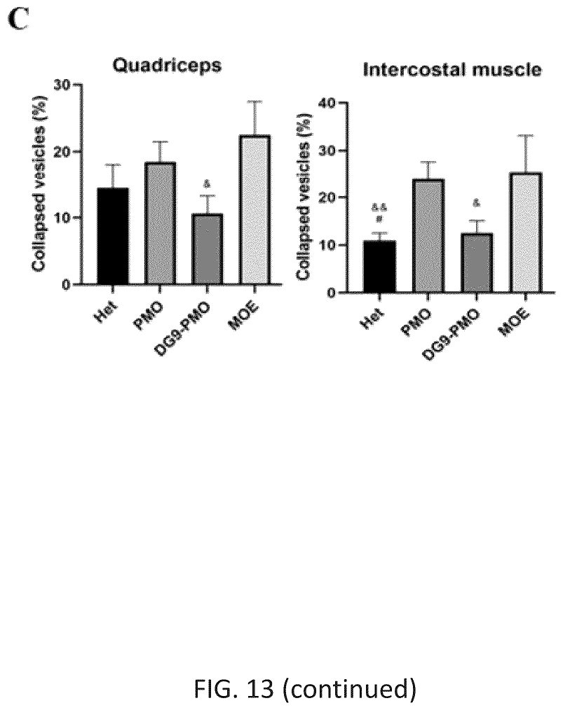

SMA mice typically begin to exhibit neuropathological deficits around PD4-PD5, with denervated and collapsed neuromuscular junctions (NMJs) as the disease progresses [37]. The NMJ architecture of the quadriceps and the intercostal muscles at PD30 was assessed to understand the phenotypic rescue following our injections at PD0 (FIG. 13a). DG9-PMO treatment restored the integrity of the NMJ, increased the endplate size, reduced denervation, and exhibited innervation patterns similar to the Hets (FIG. 13a-c). The peripheral synapses in DG9-PMO treated muscles revealed more than 60% full innervation, while unconjugated-PMO and MOE treatments had close to 40-50% innervation (FIG. 13b). Unconjugated-PMO-treated mice exhibited smaller endplates and collapsed vesicles, especially in the intercostal muscle (FIG. 11c). These data indicates that DG9-PMO treatment ameliorates the phenotype of the NMJs in SMA mice. The findings corroborate our hypothesis that DG9-PMO treatment rescues the crosstalk between the muscle and the neurons by improving muscle pathology and the characteristics of NMJs.

### DG9 Peptide Increases the Uptake of PMO in Both Systemic and CNS Tissues Following a Single Subcutaneous Administration

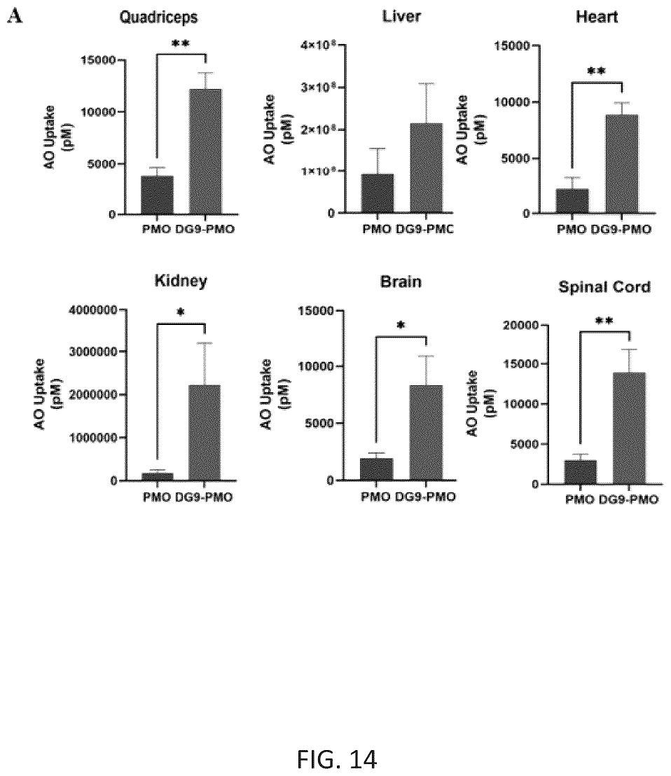

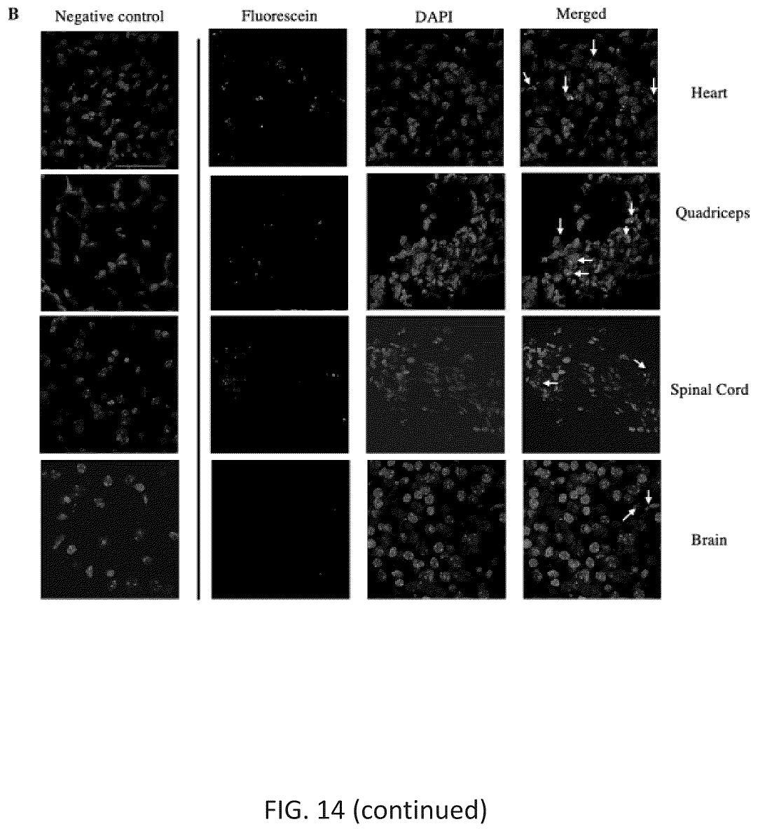

To determine the effects of DG9 peptide in increasing the PMO uptake, we evaluated the biodistribution of unconjugated-PMO and DG9-PMO in the peripheral and CNS tissues [82]. We used a well-established hybridization-based ELISA assay designed specifically to detect the PMO to compare the concentration of DG9-PMO and unconjugated-PMO in the tissues of treated PD7 mice [82]. Conjugation of DG9 to the PMO led to a significantly higher uptake in most tissues except for the liver, with an average AO detection of 12202 μM (quadriceps muscle, (n=5)), 2×10⁸ pM (liver (n=5)), 8359 μM (brain (n=5)), 13907 μM (spinal cord (n=4)), 8886 μM (heart (n=4)), and 2×10⁷ μM (kidney (n=6)) (FIG. 14a). Both unconjugated-PMO and DG9-PMO displayed higher levels in the liver and kidney, which are responsible for metabolizing and clearing PMO.

PMO uptake is mediated by the caveolin-dependent pathway in myotubes [83]. Some CPP-PMOs often get trapped in endosomes, limiting the efficiency of splicing correction [84]. To demonstrate intracellular localization of DG9-PMO, we attached a fluorescent tag to the DG9-PMO. SMA mice were subcutaneously injected at PD0 (40 mg/kg) and performed immunohistochemistry (IHC) to examine the localization of DG9-PMOs in frozen tissue sections collected at PD7. Some DG9-PMOs localized in nuclei of cells in the hearts and quadriceps muscle, and to a lesser extent in the CNS tissues (FIG. 14b). This experiment shows that DG9 promotes uptake of PMO in both the peripheral and CNS tissues following a single subcutaneous administration, thereby globally increasing SMN levels and ameliorating the SMA phenotype.

### DG9-PMO Penetrates the Blood-Brain Barrier in a Mild SMA Model

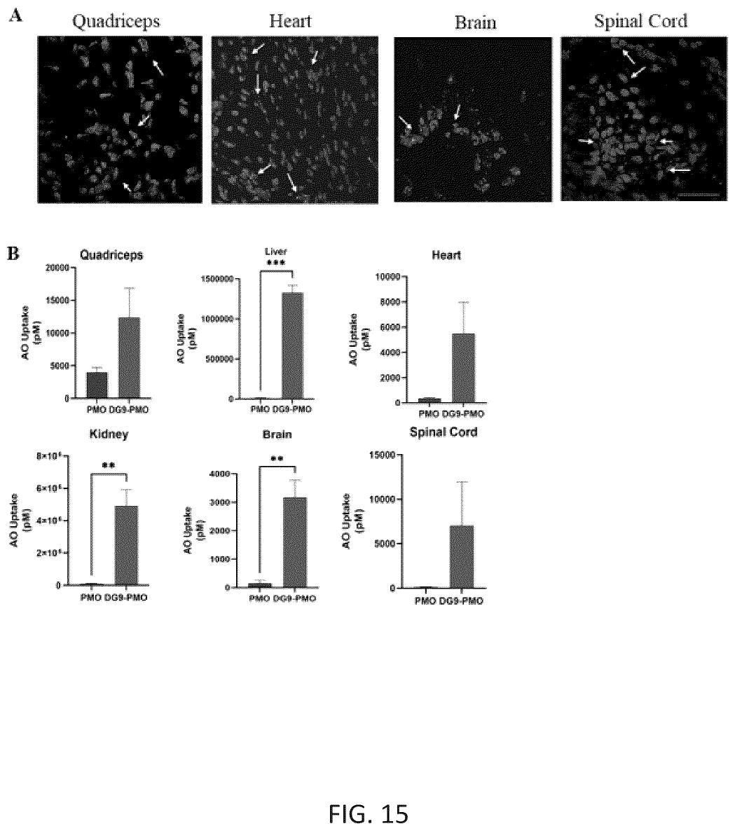

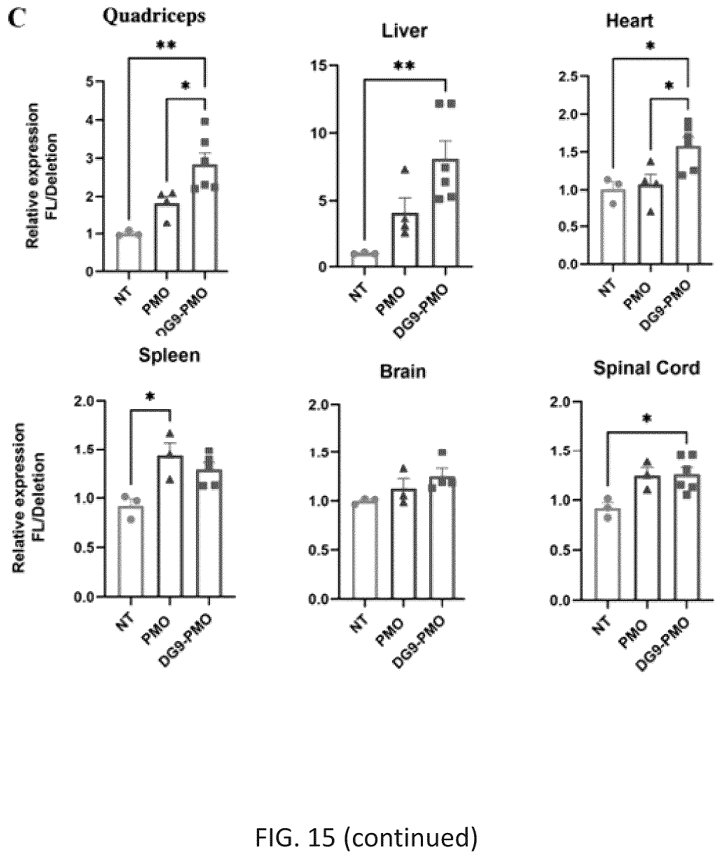

To investigate whether the DG9-PMO can cross the fully developed BBB, a milder SMA model (F0) (Smn^{-/-} SMN2^{+/+}) was injected at PD5 with DG9-PMO or fluorescently tagged DG9-PMO. These mice have a normal life span and usually only exhibit necrosis of the tail and ears around 8-12 weeks of age. Tissues were harvested at PD7. First, we observed localization of fluorescently tagged DG9-PMO inside the nuclei of the cells in both peripheral and CNS tissues (FIG. 15a). It supports our hypothesis that DG9-PMO can penetrate the BBB. An ELISA was performed to get the biodistribution of unconjugated-PMO and DG9-PMO. Despite subcutaneous administration at PD5, DG9 significantly increased uptake of PMO in the CNS and peripheral tissues (FIG. 15b). Ultimately, the FL-SMN2 levels normalized to saline-injected mice (NT control) were analyzed and an increase in the FL-SMN2 expression in the spinal cord and other peripheral tissues (FIG. 15c) was observed. These findings demonstrates that the DG9-PMO can penetrate the fully developed BBB and ensure widespread distribution of the AOs to both the peripheral and CNS tissues.

DG9-PMO Treatment Rescues the SMA Phenotype without Apparent Toxicity

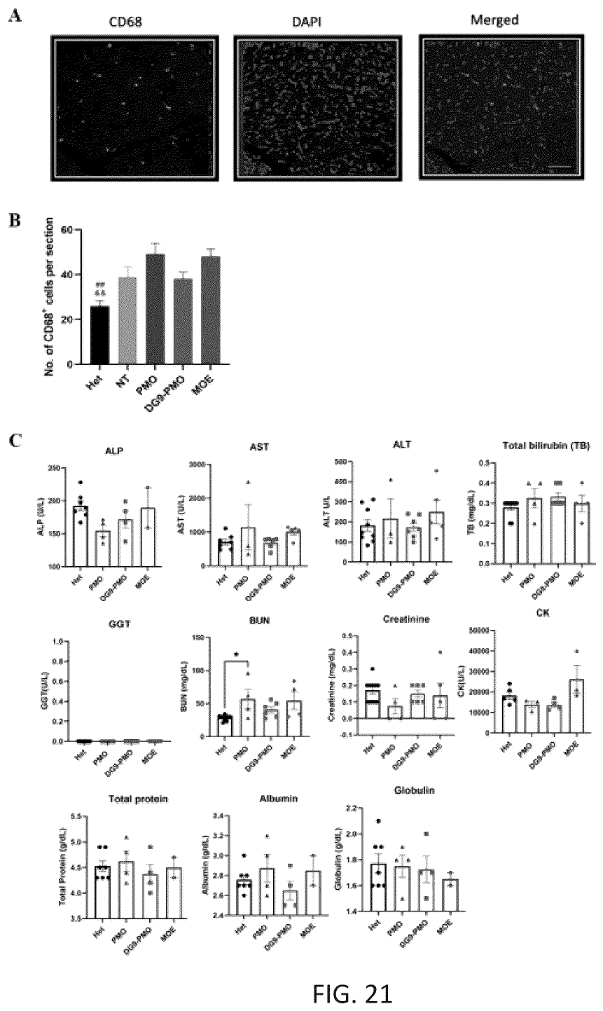

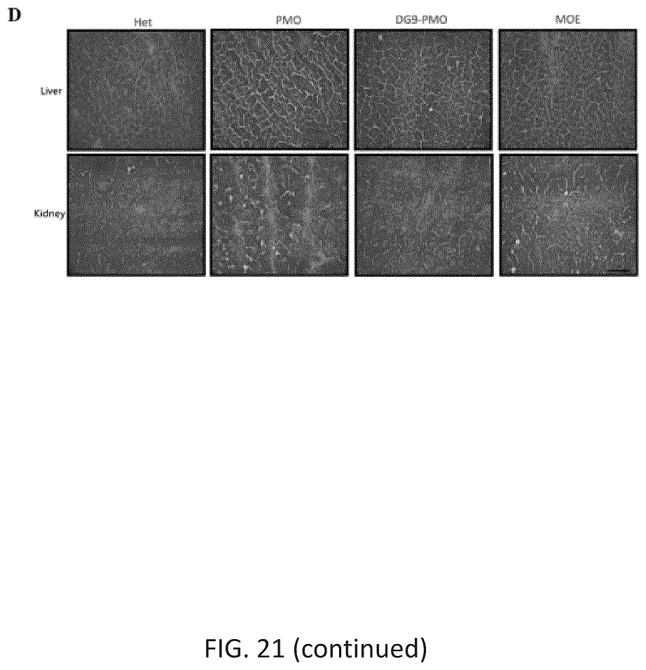

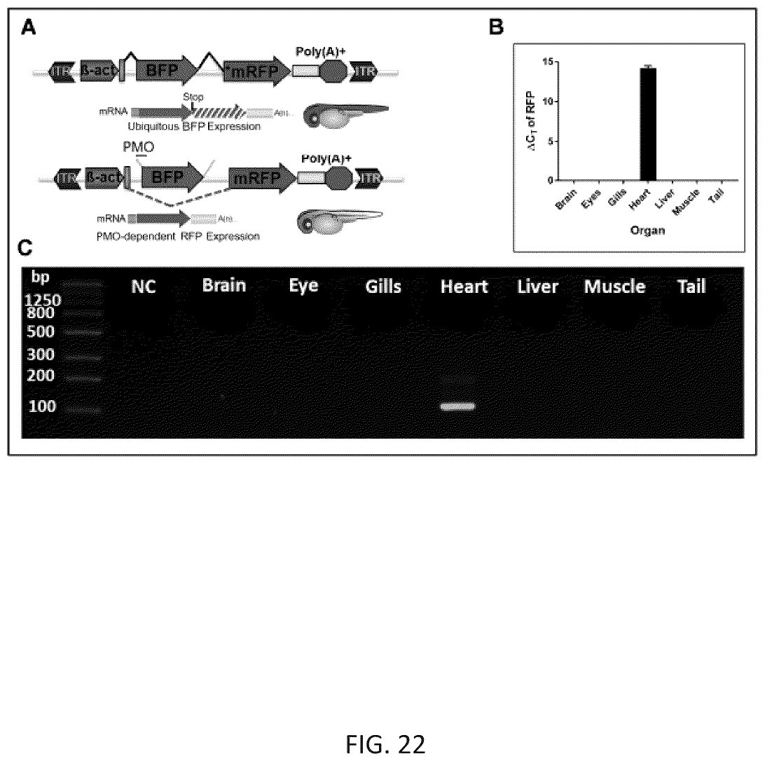

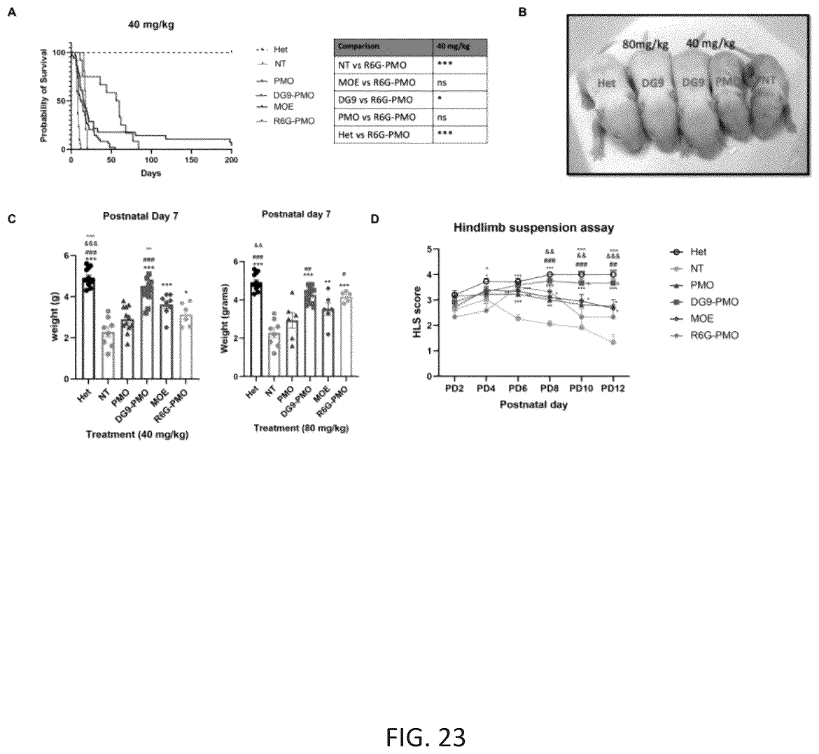

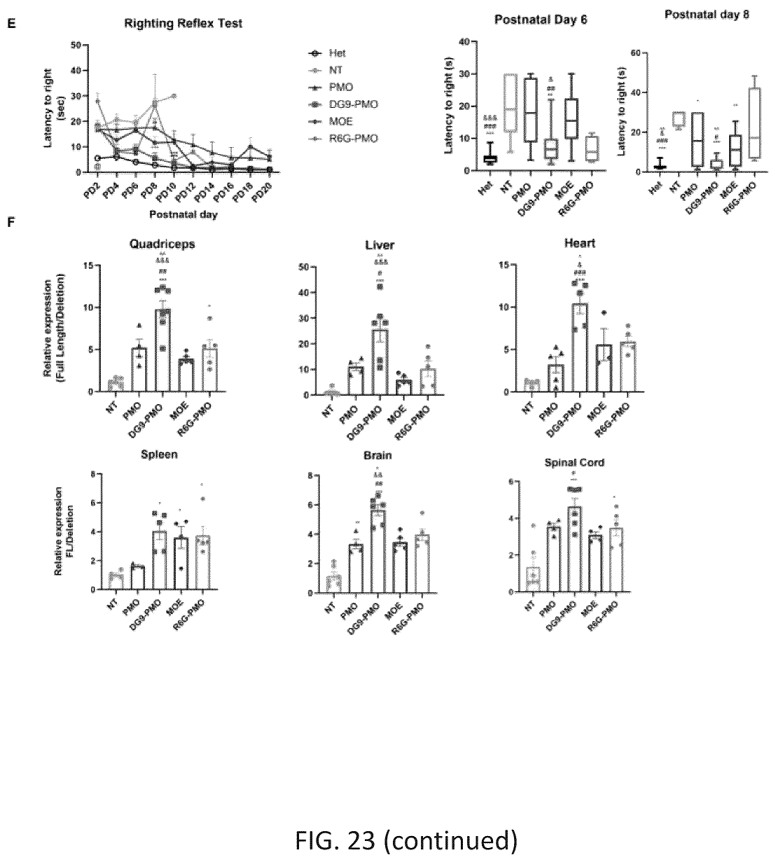

Peptides can typically pose as antigens, leading to immune reactions. Therefore, we examined the susceptibility of DG9-PMO to cause immune activation at an early neonatal stage, by looking at CD68 positive cells (CD68+), indicative of circulating and tissue macrophages in the quadriceps muscle sections at PD7 (FIG. 21a). The NT control mice exhibited elevated levels of CD68⁺ macrophages owing to the atrophic musculature in SMA.

The unconjugated-PMO and MOE-treated muscle had a significantly higher number of CD68⁺ macrophages when compared to the Hets, while NT and DG9-PMO mice had no significant difference (FIG. 21a). The apparent reduction in circulating macrophages following DG9-PMO treatment is likely due to amelioration of the atrophic musculature which would compensate for any elevation seen from the treatment itself.

To further elucidate the possibility of long-term toxicity, serum from the Hets and treated mice at PD 30-35 was collected. A toxicological evaluation was performed on the levels of alkaline phosphatase (ALP), alanine transaminase (ALT), aspartate aminotransferase (AST), creatine kinase (CK), creatinine, total bilirubin, total protein, albumin, globulin, and gamma-glutamyl transferase (GOT). All examined indicators were comparable between the groups, suggesting no apparent toxic effects (FIG. 21b). We also performed a qualitative histological analysis of the liver and kidney and found no signs of observable toxicity (FIG. 21c). These findings emphasize that DG9-PMO is associated with no apparent toxicity in mice and immune dysfunction.

### Discussion

Duplications and mutations in the telomeric SMN1 gene led to the formation of its paralog centromeric SMN2 gene with a difference in only a few nucleotides [85]. Since SMN2 can produce a small amount of functional SMN, it is a viable target for therapy in SMA patients. 95% of SMA patients have mutations in the SMN1 gene, making SMN2 gene a candidate for AO therapy to treat almost all the SMA patients.

We deployed a CPP conjugated to an AO (PMO) because some of the studied CPPs can deliver biological cargos into the cell and can also cross the BBB [86,73]. Several peptides also have demonstrated neuroprotective effects, minimized the risk of toxicity and adverse effects, making them suitable for clinical applications [87]. With several concerns still associated with nusinersen, a therapy addressing the invasive administrations, immune reactions, toxicity, and other adverse events is still needed for SMA treatment.

In this study, we evaluated the efficacy of a novel peptide DG9, identified from screening several peptides in the zebrafish model system [75]. To overcome invasive intrathecal injections and to ensure body-wide restoration of the SMN protein, we subcutaneously injected neonatal SMA mice with 40 mg/kg of DG9-PMO, unconjugated-PMO, MOE, or saline at PD0. DG9-PMO treatment significantly prolonged survival, increased bodyweights, FL-SMN2 expression, and SMN levels in both the peripheral and CNS tissues.

DG9-PMO treated neonatal and adult mice showed robust improvement in muscle strength and coordination, with significant improvement in the functional tests. We also observed a dose-dependent increase in survival and in the FL-SMN2 expression when mice were administered a higher dose of 80 mg/kg of the AOs, with DGY-PMO treated mice maintaining an improvement in motor function even at PD60.

The DG9 peptide led to sustained delivery of PMO to the skeletal and respiratory muscles that, in turn, reduced the number of myofibers with central nuclei and increased the myofiber size. At PD30, the unconjugated-PMO mice had significantly smaller myofibers in both the intercostal and quadriceps muscles, hinting at a possibility of compromised breathing, and atrophic myofibers being the cause of early mortality. Though born normally, SMA mice begin to demonstrate decreased respiratory rates and an increase in apnea frequencies by PD7, typically due to weakening of the inspiratory and expiratory muscles [88]. Our plethysmography recordings at PD7 under normoxic and hypoxic conditions demonstrate the weak, slow, and irregular breathing in NT SMA mice. We did not perform statistics to compare treatments alone, but when compared to the Hets, half the PMO mice exhibited a severe respiratory phenotype with irregular breathing and apneas, while most of the MOE mice exhibited a normal breathing function. The variability in the results, especially in unconjugated-PMO mice points towards a deeper understanding of respiratory dysfunction in SMA, leading to their abrupt mortality. Under both normoxic and hypoxic conditions, DG9-PMO mice were similar to the Hets, with significantly better respiratory outcomes than NT PD7 mice. These findings provide evidence for the efficiency of DG9-PMO in rescuing early respiratory dysfunction in SMA mice.

At PD30, there was an improvement in the NMJ architecture with fewer collapsed structures and increased innervation patterns in the DG9-PMO mice. The unconjugated-PMO mice, on the other hand, had a higher number of denervated endplates and collapsed NMJs, suggesting an overt SMA phenotype. NMJ and motor neuron development go hand-in-hand, even though the link is not strongly established. Defects in NMJs are an early pathology in motor neuron diseases and require SMN for proper innervation [81,89,90]. Our treatment improved NMJ innervation, which correlates inversely with muscle atrophy [94], thereby leading to better performance in the functional tests.

Both peripheral and CNS tissues demonstrated a significantly higher uptake of PMO when conjugated to the DG9 peptide compared to unconjugated-PMO alone, which serves as evidence for the peptide to ensure widespread distribution of the AOs even to the brain and spinal cord.

Since our mouse model has a median survival of only 8 d, the precise efficiency of treatment at a late-onset symptomatic phase cannot be completely elucidated. To demonstrate the ability of DG9 to penetrate the fully developed BBB in mice, we used a milder model (Smn^{-/-}; SMN2^{Tg/Tg}) with a normal lifespan. We observed robust uptake of PMO and localization of the DG9 peptide in the CNS tissues following a single subcutaneous injection at PD5. However, from a therapeutic standpoint, it is necessary to corroborate our findings in detail in other mild SMA models with multiple copies of SMN2 that could serve as models to treat patients with type III-IV late-onset SMA or in type I-II at a symptomatic clinical stage.

Toxicity associated with cationic peptide conjugated PMOs is typically seen within 24 hours of administration. No apparent physiological differences in the neonates receiving subcutaneous injections of the AOs. They were active, similar to their healthy counterparts. We also quantified the number of CD68⁺ cells in quadriceps muscle at PD7, that are indicative of macrophages activated during an immune response in the body upon administration of AOs. SMA mice suffer from immune dysregulation and have an elevated number of immune cells. While DG9-PMO and NT mice had similar numbers of CD68⁺ cells, we believe that although DG9-PMO treatment might evoke an immune response, the simultaneous improvement in muscle pathology and amelioration of the atrophic muscles reduced the total number of circulating macrophages. No apparent toxicity in the liver or kidney as per the serum and histology analyses at PD30 was observed.

### Material and Methods

### Synthesis of DG9-PMO

DG9 (sequence N-YArVRRrGPRGYArVRRrGPRr-C; uppercase: L-amino acids, lowercase: D-amino acids) was synthesized and covalently conjugated to the 3′ end of the PMO. The PMO targeting ISS-N1 intron 7 (5′-TCACTTTCATAATGCTGG-3′) was purchased from Gene Tools LLC. The 2′MOE was purchased from Eurogentec North America, USA.

### Animal Models

All animal experiments were conducted in University of Alberta and approved by the Animal Care and Use Committee, University of Alberta Research Ethics Office. SMA transgenic mice (JAX stock #005058 FVB.Cg-Tg(SMN2)2HungSmn1^{tm1Hung}/J (Smn^{-/-}; SMN2^{Tg/Tg}), also known as the Taiwanese mice) were purchased from Jackson Laboratory (Bar Harbor, ME, USA). Heterozygous mice for Smn1 (Smn1^{+/-}; SMN2^{-/-}) were crossed with mice homozygous for Smn1 Smn^{-/-}; SMN2^{Tg/Tg} to obtain the SMA mice (Smn^{-/-}; SMN2^{Tg/-}) or the heterozygous healthy control (Smn1^{+/-}; SMN2^{Tg/-}). The SMA mice display a severe overt phenotype similar to SMA type I patients. The mice were genotyped using a PCR assay on genomic DNA isolated from tail biopsies using the Phire Tissue Direct PCR kit (ThermoFisher) as per the manufacturer's instructions. The DNA was amplified using the primers described in Table 5 using the condition Mouse Smn: 98° C./5 min→98° C./5 s_58° C./10_72° C./10 s×35 cycles=72° C./1 min.

### Treatment

Injections were carried out using a 30-gauge Hamilton syringe. SMA neonates were subcutaneously injected with 40 or 80 mg/kg of AOs at P0, while NT and heterozygous control mice were injected with saline (n=10-25) per group. Tissues including the quadricep muscle, liver, kidney, spleen, diaphragm, intercostal muscle, heart, brain and spinal cord were collected and snap frozen in dry-ice cooled isopentane, and then subsequently stored at −80° C.

### Real-Time Quantitative PCR (RT-qPCR)

Frozen tissues harvested at P7 were sectioned using a cryostat (Leica C M 1950, Leica) into 20 μm sections. RNA was extracted from these sections using TRizol 10 Reagent (Invitrogen). cDNA was synthesized from 50 ng/μl RNA using superscript IV Reverse Transcriptase (ThermoFisher) and oligo(dT) primers (ThermoFisher) following the manufacturer's instructions. qPCR reaction was performed using the SsoAdvanced Universal SYBR Green Supermix (BioRad), and QuantStudio3 real-time PCR system (Applied Biosystems). The relative gene expression for full-lengthSMN2 over the deletion SMN2 transcripts without exon 7 was normalized to the NT control samples and analyzed using the ΔΔCt method.

### Western Blotting

Total protein was extracted from frozen tissues harvested at P7 or P30 by using RIPA buffer (Sigma) with cOmplete, Mini, EDTA-free protease inhibitor cocktail (Sigma). The protein concentration was quantified using the Pierce BCA Protein Assay Kit (ThermoFisher). For SDS-PAGE, 5-10 μg of protein per well was run per well in NuPAGE Novex 4-12% Bis-Tris Midi protein gels (Life Technologies) at 150 V for 60 minutes, followed by semi-dry transfer at 20 V for 30 minutes. The polyvinylidene fluoride (PVDF) membrane was blocked overnight with 5% skim milk and 0.05% Tween20 in PBS (PBST). The membrane was incubated with a mouse purified anti-SMN antibody (BD Biosciences) (1:10,000) for one hour at room temperature (RT) under agitation. The membrane was washed three times with PBST (10 minutes each wash) and incubated with HRP conjugated goat anti-mouse (IgG H+L) at RT for 1 hour under agitation. The bands were detected using the Amersham ECL Select Western Blotting Detection Kit (GE Healthcare) and visualized by ChemiDoc Touch Imaging system (BioRad). For β-tubulin, the membrane was incubated with the stripping buffer (15 g glycine, 1 g SDS, 10 nil Tween20 at pH 2.2) for 10 minutes at RT, followed by two washes in PBS (10 minutes each) and two washes in tris-buffered saline and 0.05% Tween20 (TBST) (5 minutes each). Similar to the primary antibody protocol, the membrane was subsequently blocked overnight and incubated with β-tubulin rabbit antibody (Abcam ab6046, 1:5000) at room temperature for 1 hour under agitation the next day. The secondary antibody used was HRP conjugated goat anti-rabbit (IgG H+L) for Tubulin (BioRad), 1:10,000). The bands were visualized as mentioned previously.

### Enzyme-linked Immunosorbent Assay (ELISA)

ELISA was performed as described previously [83,96]. In brief, protein was extracted from frozen tissue sections (˜20 μm) using RIPA buffer (Sigma) with cOmplete, Mini, EDTA-free protease inhibitor cocktail (Sigma). The probes (Integrated DNA Technologies) were designed complementary to the PMO sequence with phosphorothioated backbones at the 5′ and 3′ ends. The 5′ and 3′ ends were labelled with digoxigenin and biotin, respectively. The tissue lysates (0.02 mg/ml protein concentration) were pre-treated with 2.5 mg/mL trypsin containing 10 mM CaCl₂ at 37° C. overnight to digest the DG9 peptide. The probes were added to the samples and allowed to hybridize at 37° C. for 30 minutes. Following the probe-PMO hybridization, the hybridized samples were added to Pierce NeutrAvidin Coated 96-Well Plates, Black (Thermo Fisher Scientific) to allow avidin-biotin interaction between the plate and the probes. The unhybridized probes were digested using micrococcal nuclease enzyme at 0.1 gel unit/pi (New England Biolabs). This was followed by the addition of anti-digoxigenin antibody conjugated with alkaline phosphatase (1:5000, Roche Applied Sciences). Attophos AP Fluorescent Substrate (Promega) was added to the PMO/DG9-PMO probes and fluorescence was detected at 444 nm excitation and 555 nm emission by using a rnonochromator SpectraMax M3 plate reader (Molecular Devices).

### Histology and Immunohistochemistry

### H and E Staining

7 μm cryosections of the quadricep muscle, diaphragm and intercostal muscle were stained with Meyer's H&E reagents (Electron Microscopy Reagents) [97]. For the centrally nucleated fibers and myofiber size, 500-800 were randomly selected per muscle per treatment. The cross-sectional area and minimal Feter's diameter were quantified using Image J. All H and E analyses were performed in a blinded fashion.

### NMJ Staining

NMJ staining was carried out as previously described [98]. Briefly, the quadricep and the intercostal muscle were fixed in 4% paraformaldehyde (PFA) for 2 hours. The muscles were washed quicky with PBS and blocked with 5% goat serum and 2% Triton-X in PBS for 1 hour at RT. This was followed by an overnight incubation at 4 with the primary mouse monoclonal antibodies anti-neurofilament 2H3 (1:100) and anti-synaptophysin (1:500) (DSHB, Iowa). Subsequently, the muscles were incubated in the dark with Alexafluor 488-goat anti-mouse IgG1 (1:1000) (Life Technologies) and Alexafluor 594-bungarotoxin (1:5000) (ThermoFisher Scientific). At least 300 NMJs were z-stacked and visualized for the analysis of denervation and cross-sectional area using a confocal microscope (Zeiss LSM 710) and Zeiss Zen software. The synaptic area was quantified using image J. All analyses were performed in a blinded fashion.

### Macrophage Detection

7 μm cryosections of the quadricep muscle were fixed with 4% PFA. The sections were incubated with rat anti-mouse CD68 antibody (Bio-Rad, MCA1957T). The number of CD68⁺ cells were counted and averaged from at least 5 sections per sample at random intervals at 20× magnification.

### Functional Tests

### Righting Reflex Test

This spontaneous ability of the mice to right themselves up was tested from P2 until the weaning stage P20. The neonates were placed on their backs and the time taken to reposition and place all four paws on the ground was noted. The recording time was a maximum of 60 s. Each neonate underwent three trials, with a resting period of at least 10 minutes between trials. The data is expressed as the mean time to complete the righting reflex test ±SEM.

### Hindlimb Suspension Assay (Tube Test)

This test was performed on neonatal mice from P2-P12 as described in Treat NMD protocol SMA_M.2.2.001. Briefly neonatal mice were suspended on their hindlimbs from a tube. They were scored based on the position of the hindlimbs and their latency to fall was recorded with a 30 second cut-off. Each neonatal pup underwent three trials, with a 15-minute break between each trial. The average score was noted down by the observer who was blinded to this test.

### Forelimb Grip Strength

This assay was conducted as described in Treat NMD protocol SMA_M.2.1.002. In short, the mouse is placed on the wire mesh of an automated grip strength meter (Columbus Instruments) such that only the front paws are allowed to grip the metal grid. The mouse is steadily pulled with the help of its tail, such that it lets go of the grid completely. P30 and P60 mice were used. Each mouse underwent three trials.

### Rotarod Test

The rotarod test (AccuScan Instruments) was performed on mice between P30-P40, using an acceleration profile of 300s as previously described [99].

### Toxicology

Blood was collected from the mice around P30-P40 (n=4-10 per group) during the dissection procedure. The blood was left at RT for 30 minutes and centrifuged at 2000 rcf. The resulting supernatant (serum) was transferred to a new 1.5 ml tube and stored at −20° C. The serum samples were analyzed by Idexxx Bioanalytics. The evaluation was performed on a standard set of toxicity markers: glucose, total bilirubin, blood urea nitrogen (BUN), creatine kinase (CK), creatinine, alkaline phosphatase (ALP), alanine transaminase (ALT), aspartate aminotransferase (AST), gamma-glutamyl transferase (GGT), globulin, albumin, and total protein.

### Whole-Body Plethysmographic Recordings

Measurements were performed in whole body, cylindrical transparent plexiglass plethysmographs that had one inflow and two outflow ports for the continuous delivery of fresh room air and removal of expired carbon dioxide [100,101]. The plethysmograph of volumes were 10 ml (inner diameter: 1.9 cm, length: 3.5 cm for P7 mice with body weight less than 2.5 g), 30 ml (inner diameter: 2.6 cm, length: 5.6 cm for P7 mice with body weight more than 2.5 g) and 80 ml (inner diameter: 3.8 cm, length: 7 cm for P30) for measures of respiratory parameters with a flow rate of 15, 45, 120 ml/min, respectively. The gas was mixed with gas mixer (GSM-3, CWE InC, USA), delivered from compressed pure oxygen and pure nitrogen cannisters, being monitored using 0-200 ml/min gas regulators (Porter Instrument Company, USA).

Hypoxic challenge (11% of oxygen for 5 min) was performed with continuous monitoring of plethysmographic recordings without physical handling of the animal by switching inflow gas from normoxia (21% of oxygen, balanced by nitrogen) to hypoxia (11% of oxygen, balanced by nitrogen). It took about 1 min to finish gas exchange, confirmed with gas analyzer (Model: ML206, ADlnstruments). For P7, the plethysmograph was contained within an infant incubator (Isolette, model C-86: Air-Shields/Drager Medical, USA) to maintain the ambient temperature at the approximate nest temperature of 32° C. For P30, the plethysmograph was recorded at room temperature of around 22° C. Pressure changes were detected with a pressure transducer (model DP 103; Validyne, USA), signal conditioner (CD-15; Validyne), recorded with data acquisition software (Axoscope) via analog-digital board (Digidata 1322A). Signals were high pass filtered (0.01 kHz), with a sampling rate at 1 kHz. Respiratory frequency and tidal volume (V_{T}) was measured with blood pressure settings using Labchart 8 (AD Instruments Inc., USA). Threshold levels for bursts were set, and bursts were then automatically detected so that frequency (calculated from cycle duration) and tidal volume (calculated from maximal pressure minus minimal pressure).

It should be noted that our plethysmograph is effective for studying respiratory frequency (f_{R}) and detection of apneas. An apnea is defined as the absence of airflow (pressure changes) for a period equivalent or greater than two complete respiratory cycles. Our whole-body plethysmographic system provided semiquantitative measurements of tidal volume (VT, mL/g) and minute ventilation, from which we report changes relative to the wildtype normoxia (Ren et al., 2009, 2015). The coefficient of variation of frequency, a measure of relative variability, is the ratio of the standard deviation to the mean (average). The smaller the ratio, the more regular the breathing. The experiments were conducted between 10 am and 5 pm. Animals were sent back to the animal facility after experiments.

Respiratory parameters were calculated over an average of 1 min of continuous plethysmography recordings. The respiratory parameters V_{T} and V_{E} were reported relative to the mean of heterozygotes in normoxia (100%). The nature of the hypothesis testing is two-tailed. We first ran the normality test (Shapiro-Wilk) and equal variance test (Brown-Forsythe).

For those data (FIG. 6B-E: f_{R}, V_{T}, V_{E}, and CV) that passed both tests, parametric statistics were used with two-way repeated measures analysis of variation (ANOVA), followed by Holm-Sidak method (two factors: different treatments and different conditions). p<0.05 is taken as a statistically significant difference: n refers to the number of animals, with animal as the unit of analysis for statistical tests. For those data (total apnea duration, FIG. 6F) that failed either the normality test or equal variance test, nonparametric statistics were applied. Comparison of the difference in normoxia, or hypoxia was conducted with kruskal-Wallis one-way ANOVA on ranks, followed by Dunn's method. The difference between hypoxia and normoxia was conducted with a signed rank test. To examine if severity of respiratory phenotype (decrease of respiratory frequency or V_{E}) was correlated with decrease of body weight, we used the Pearson product moment correlation t test (FIG. 6G). For whole-body plethysmography recordings, data are expressed as mean±SD, or first interquartile 25%, median 50%, and third interquartile 75% (Sigmaplot 11 Systat Software Inc., USA).

### Statistical Analyses

All statistical analyses for all data (except respiratory analysis) were performed with GraphPad Prism 9 software. One-way ANOVA with Tukey's test for mUltiple comparisons, or long-rank Mantel-Cox test for survival analysis were used as needed. No statistical power calculation was conducted before the study. The sample size was based on our previous experience with these experimental protocols.

TABLE 5
List of primers used in this study

| Target | Sequence (5′-3′) |
|---|---|
| Smn-S1 | ATAACACCACCACTCTTACTC |
| Smn-S2 | GTAGCCGTGATGCCATTGTCA |
| Smn-H1 | AGCCTGAAGAACGAGATCAGC |
| FL-SMN2 | F: GCTATCATACTGGCTATTATATGGGTTTT |
| | R: CTCTATGCCAGCATTTCTCCTTAAT |
| ΔSMN2 | F: TCTGGACCACCAATAATTCCCC |
| | R: ATGCCAGCATTTCCATATAATAGCC |
| mGAPDH | F: GAGAAACCTGCCAAGTA |
| | R: CAGTGTAGCCCAAGATG |

### REFERENCES

1. Hoffman E P, Brown R H, Kunkel L M (1987) Dystrophin: the protein product of the Duchenne muscular dystrophy locus. Cell 51(6):919-928.
2. Petrof B J, Shrager J B, Stedman H H, Kelly A M, Sweeney H L (1993) Dystrophin protects the sarcolemma from stresses developed during muscle contraction. Proc Natl Acad Sci USA 90(8):3710-3714.
3. Manzur A, Kinali M, Muntoni F (2008) Update on the management of Duchenne muscular dystrophy. Arch Dis Child 93(11):986-990.
4. Crisafulli S, et al. (2020) Global epidemiology of Duchenne muscular dystrophy: an updated systematic review and meta-analysis. Orphanet J Rare Dis 15(1):141.
5. Bladen C L, et al. (2015) The TREAT-NMD DMD Global Database: Analysis of More than 7,000 Duchenne Muscular Dystrophy Mutations. Hum Mutat 36(4):395-402.
6. Monaco A P, Bertelson C J, Liechti-Gallati S, Moser H, Kunkel L M (1988) An explanation for the phenotypic differences between patients bearing partial deletions of the DMD locus. Genomics 2(1):90-95.
7. Lim KRQKRQ, Maruyama R, Yokota T (2017) Eteplirsen in the treatment of Duchenne muscular dystrophy. Drug Des Devel Ther 11:533-545.
8. Anwar S, Yokota T (2020) Golodirsen for Duchenne muscular dystrophy, Drugs of Today 56(8):491-504.
9. Roshmi, R R, Yokota T (2019) Viltolarsen for the treatment of Duchenne muscular dystrophy. Drugs of Today 55(10):627.
10. Shirley M (2021) Casimersen: First Approval. Drugs. doi:10.1007/s40265-021-01512-2.
11. Aartsma-Rus A, et al. (2009) Theoretic applicability of antisense-mediated exon skipping for Duchenne muscular dystrophy mutations. Hum Mutat 30(3):293-299.
12. Béroud C, et al. (2007) Multiexon skipping leading to an artificial DMD protein lacking amino acids from exons 45 through 55 could rescue up to 63% of patients with Duchenne muscular dystrophy. Hum Mutat 28(2):196-202.
13. Echigoya Y, et al. (2019) Exons 45-55 Skipping Using Mutation-Tailored Cocktails of Antisense Morpholinos in the DMD Gene. Mol Ther 27(11):2005-2017.
14. Lim K R Q, Nguyen Q, Yokota T (2020) Genotype-Phenotype Correlations in Duchenne and Becker Muscular Dystrophy Patients from the Canadian Neuromuscular Disease Registry. J Pers Med 10(4):241.
15. Nakamura A, et al. (2017) Comparison of the phenotypes of patients harboring in-frame deletions starting at exon 45 in the Duchenne muscular dystrophy gene indicates potential for the development of exon skipping therapy. J Hum Genet 62(4):459-463.
16. Aoki Y, et al. (2012) Bodywide skipping of exons 45-55 in dystrophic mdx52 mice by systemic antisense delivery. Proc Natl Acad Sci USA 109(34):13763-13768.
17. Echigoya Y, Mouly V, Garcia L, Yokota T, Duddy W (2015) In Silico Screening Based on Predictive Algorithms as a Design Tool for Exon Skipping Oligonucleotides in Duchenne Muscular Dystrophy. PLoS One 10(3):e0120058.
18. Lim K R Q, et al. (2019) Efficacy of Multi-exon Skipping Treatment in Duchenne Muscular Dystrophy Dog Model Neonates. Mol Ther 27(1):76-86.
19. Lehto T, et al. (2014) Cellular trafficking determines the exon skipping activity of Pip6a-PMO in mdx skeletal and cardiac muscle cells. Nucleic Acids Res 42(5):3207-3217.
20. Tsoumpra M K, et al. (2019) Peptide-conjugate antisense based splice-correction for Duchenne muscular dystrophy and other neuromuscular diseases. EBioMedicine 45:630-645.
21. Kim J, Clark K, Barton C, Tanguay R, Moulton H (2018) A Novel Zebrafish Model for Assessing In Vivo Delivery of Morpholino Oligomers, pp 293-306.
22. Veltrop M, et al. (2018) A dystrophic Duchenne mouse model for testing human antisense oligonucleotides, PLoS One 13(2):e0193289.
23. Yavas A, et al. (2020) Detailed genetic and functional analysis of the hDMDdel52/mdx mouse model. PLoS One 15(12):e0244215.
24. Sicinski P, et al. (1989) The molecular basis of muscular dystrophy in the mdx mouse: a point mutation. Science 244(4912):1578-1580.
25. Suzuki H, et al. (2016) Endogenous Multiple Exon Skipping and Back-Splicing at the DMD Mutation Hotspot. Int J Mol Sci 17(10):1722.
26. Echigoya Y, et al. (2017) Quantitative Antisense Screening and Optimization for Exon 51 Skipping in Duchenne Muscular Dystrophy. Mol Ther. doi:10.1016/j.ymthe.2017.07.014.
27. Choi J-M, et al. (2006) Intranasal delivery of the cytoplasmic domain of CTLA-4 using a novel protein transduction domain prevents allergic inflammation. Nat Med 12(5):574-579.
28. Lehto T, Ezzat K, Wood M J A, El Andaloussi S (2016) Peptides for nucleic acid delivery. Adv Drug Deliv Rev 106(Pt A):172-182.
29. Moulton H M, Moulton J D (2010) Morpholinos and their peptide conjugates: Therapeutic promise and challenge for Duchenne muscular dystrophy. Biochim Biophys Acta-Biomnenbr 1798(12):2296-2303,
30. Wu R P, et al. (2007) Cell-penetrating peptides as transporters for morpholino oligomers: effects of amino acid composition on intracellular delivery and cytotoxicity. Nucleic Acids Res 35(15):5182-5191.
31. Aoki Y, et al. (2010) In-frame dystrophin following exon 51-skipping improves muscle pathology and function in the exon 52-deficient mdx mouse. Mol Ther 18(11):1995-2005.
32. Aupy P, et al. (2020) Long-Term Efficacy of AAV9-U7snRNA-Mediated Exon 51 Skipping in mdx52 Mice. Mol Ther—Methods Clin Dev 17:1037-1047.
33. McCourt J L, et al. (2015) In vitro stability of therapeutically relevant, internally truncated dystrophins. Skelet Muscle 5(1):13.
34. Spitali P, et al. (2013) DMD transcript imbalance determines dystrophin levels. FASEB J 27(12):4909-4916.
35. Anthony K, et al. (2014) Biochemical Characterization of Patients With In-Frame or Out-of-Frame DM0 Deletions Pertinent to Exon 44 or 45 Skipping. JAMA Neurol71(1):32.
36. van Putten M, et al. (2012) The Effects of Low Levels of Dystrophin on Mouse Muscle Function and Pathology. PLoS One 7(2):e31937.
37. Kudoh H, et al. (2005) A new model mouse for Duchenne muscular dystrophy produced by 2.4 Mb deletion of dystrophin gene using Cre-loxP recombination system. Biochem Biophys Res Commun 328(2):507-516.
38. Aartsma-Rus A, van Putten M (2014) Assessing Functional Performance in the Mdx Mouse Model. J Vis Exp (85):51303.
39. Lim K R Q, Yokota T (2018) Quantitative evaluation of exon skipping in immortalized muscle cells in vitro. Exon Skipping and Inclusion Therapies: Methods and Protocols, eds Yokota T, Maruyama R Springer, New York), pp 127-140.
40. Maruyama R, et al. (2017) Systemic Delivery of Morpholinos to Skip Multiple Exons in a Dog Model of Duchenne Muscular Dystrophy. Methods Mol Biol 1565:201-213.
41. Thanh L T, et al. (1995) Characterization of genetic deletions in becker muscular dystrophy using monoclonal antibodies against a deletion-prone region of dystrophin. Am J Med Genet 58(2):177-186.
42. Desgeorges T, et al. (2019) Open-OSAM, a new tool for semi-automated analysis of myofiber cross-sectional area in regenerating adult skeletal muscle. Skelet Muscle 9(1):2.
43. Landini G, Martinelli G, Piccinini F (2020) Colour deconvolution: stain unmixing in histological imaging. Bioinformatics:btaa847.
44. Ruifrok A C, Johnston D A (2001) Quantification of histochemical staining by color deconvolution. Anal Quant Cytol Histol 23(4):291-299.
45. Lunn M R, Wang C H (2008) Spinal muscular atrophy. Lancet 371 (9630):2120-2133. doi:10.1016/S0140-6736(08)60921-6
46. D'Amico A, Mercuri E, Tiziano F D et al. (2011) Spinal muscular atrophy. Orphanet J Rare Dis 6:71. doi:10.1186/1750-1172-6-71
47. Lefebvre S, Burglen L, Reboullet S et al. (1995) Identification and characterization of a spinal muscular atrophy-determining gene. Cell 80 (1):155-165. doi:10.1016/0092-8674(95)90460-3
48. Monani U R, Lorson C L, Parsons D W et al. (1999) A single nucleotide difference that alters splicing patterns distinguishes the SMA gene SMN1 from the copy gene SMN2. Hum Mol Genet 8 (7):1177-1183. doi:10.1093/hmg/8.7.1177
49. Schrank B, Gotz R, Gunnersen J M et al. (1997) Inactivation of the survival motor neuron gene, a candidate gene for human spinal muscular atrophy, leads to massive cell death in early mouse embryos. Proc Natl Acad Sci USA 94 (18):9920-9925. doi:10.1073/pnas.94.18.9920
50. Schmutz J, Martin J, Terry A et al. (2004) The DNA sequence and comparative analysis of human chromosome Nature 431 (7006):268-274. doi:10.1038/nature02919
51. Vitte J, Fassier C, Tiziano F D et al, (2007) Refined characterization of the expression and stability of the SMN gene products. Am J Pathol 171 (4):1269-1280. doi:10.2353/ajpath.2007.070399
52. Singh N K S N, Androphy E J, Singh R N (2006) Splicing of a critical exon of human Survival Motor Neuron is regulated by a unique silencer element located in the last intron. Mol Cell Biol 26 (4):13. doi: 10.1128/MCB.26.4.1333-1346.2006
53. Sumner C J, Crawford T O (2018) Two breakthrough gene-targeted treatments for spinal muscular atrophy: challenges remain. J Clin Invest 128 (8):3219-3227. doi:10.1172/JC1121658
54. Li Q (2020) Nusinersen as a Therapeutic Agent for Spinal Muscular Atrophy. Yonsei Med J 61 (4):273-283, doi:10.3349/ymj.2020.61.4.273
55. Lorson C L, Hahnen E, Androphy E J et al. (1999) A single nucleotide in the SMN gene regulates splicing and is responsible for spinal muscular atrophy. Proc Natl Acad Sci USA 96 (11):6307-6311, doi:10.1073/pnas.96.11.6307
56. Campbell L, Potter A, Ignatius J et al. (1997) Genomic variation and gene conversion in spinal muscular atrophy: implications for disease process and clinical phenotype. Am J Hum Genet 61 (1):40-50. doi:10.1086/513886
57. Hua Y, Vickers T A, Okunola H L et al. (2008) Antisense masking of an hnRNP A I/A2 intronic splicing silencer corrects SMN2 splicing in transgenic mice. Am J Hum Genet 82 (4):834-848. doi:10.1016/j.ajhg.2008.01.014
58. Hamilton G, Gillingwater T H (2013) Spinal muscular atrophy: going beyond the motor neuron. Trends Mol Med 19 (1):40-50. doi:10.1016/j.molmed.2012.11.002
59. Nash L A, Burns J K, Chardon J W et al. (2016) Spinal Muscular Atrophy: More than a Disease of Motor Neurons?Curr Mol Med 16 (9):779-792. doi:10.2174/1566524016666161128113338
60. Deguise M O, De Repentigny Y, McFall E et al. (2017) Immune dysregulation may contribute to disease pathogenesis in spinal muscular atrophy mice. Hum Mol Genet 26 (4):801-819. doi:10.1093/hmg/ddw434
61. Rudnik-Schoneborn S, Heller R, Berg C et al. (2008) Congenital heart disease is a feature of severe infantile spinal muscular atrophy. J Med Genet 45 (10):635-638. doi:10.1136/jmg.2008.057950
62. Bevan A K, Hutchinson K R, Foust K D et al. (2010) Early heart failure in the SMNDelta7 model of spinal muscular atrophy and correction by postnatal scAAV9-SMN delivery. Hum Mol Genet 19 (20):3895-3905. doi:10.1093/hmg/ddq300
63. Heier C R, Satta R, Lutz C et al. (2010) Arrhythmia and cardiac defects are a feature of spinal muscular atrophy model mice. Hum Mol Genet 19 (20):3906-3918. doi:10.1093/hmg/ddq330
64. Kolb S J, Kissel J T (2011) Spinal muscular atrophy: a timely review. Arch Neurol 68 (8):979-984. doi:10.1001/archneurol.2011.74
65. Kim J K, Jha N N, Feng Z et al. (2020) Muscle-specific SMN reduction reveals motor neuron-independent disease in spinal muscular atrophy models. J Clin Invest 130(3):1271-1287. doi:10.1172/JC1131989
66. Lowery S, Oliver A (2008) Incidence of postdural puncture headache and backache following diagnostic/therapeutic lumbar puncture using a 22G cutting spinal needle, and after introduction of a 25G pencil point spinal needle. Paediatr Anaesth 18 (3):230-234. doi:10.1111/j.1460-9592.2008.02414.x
67. Hache M, Swoboda K J, Sethna N et al. (2016) Intrathecal Injections in Children With Spinal Muscular Atrophy: Nusinersen Clinical Trial Experience. J Child Neurol 31 (7):899-906. doi:10.1177/0883073815627882
68. Hoy S M (2019) Onasemnogene Abeparvovec: First Global Approval. Drugs 79 (11):1255-1262. doi:10.1007/s40265-019-01162-5
69. Van Alstyne M, Tattoli I, Delestree N et al. (2021) Gain of toxic function by long-term AAV9-mediated SMN overexpression in the sensorimotor circuit. Nat Neurosci. doi:101038/s41593-021-00827-3
70. Singh R N, Ottesen E W, Singh N N (2020) The First Orally Deliverable Small Molecule for the Treatment of Spinal Muscular Atrophy. Neurosci Insights 15:2633105520973985. doi:10.1177/2633105520973985
71. Dhillon S (2020) Risdiplam: First Approval. Drugs 80 (17):1853-1858, doi:10.1007/s40265-020-01410-z
72. Ratni H, Ebeling M, Baird J et al. (2018) Discovery of Risdiplam, a Selective Survival of Motor Neuron-2 (SMN2) Gene Splicing Modifier for the Treatment of Spinal Muscular Atrophy (SMA). J Med Chem 61 (15):6501-6517. doi:10.1021/acs.jmedchem.8b00741
73. Sheng L, Rigo F, Bennett C F et al. (2020) Comparison of the efficacy of MOE and PMO modifications of systemic antisense oligonucleotides in a severe SMA mouse model. Nucleic Acids Res 48 (6):2853-2865. doi:10,1093/nar/gkaal26
74. Hammond S M, Hazell C, Shabanpoor F et al. (2016) Systemic peptide-mediated oligonucleotide therapy improves long-term survival in spinal muscular atrophy. Proc Natl Acad Sci USA 113 (39):10962-10967. doi:10.1073/pnas.1605731113
75. Shabanpoor F, Hammond S M, Abendroth F et al. (2017) Identification of a Peptide for Systemic Brain Delivery of a Morpholino Oligonucleotide in Mouse Models of Spinal Muscular Atrophy. Nucleic Acid Ther 27 (3):130-143. doi:10.1089/nat.2016.0652
76. Wesolowski D, Tae H S, Gandotra N et al. (2011) Basic peptide-morpholino oligomer conjugate that is very effective in killing bacteria by gene-specific and nonspecific modes. Proc Natl Acad Sci USA 108 (40):16582-16587. doi:10.1073/pnas.1112561108
77. Choi J M, Ahn M H, Chae W J et al. (2006) Intranasal delivery of the cytoplasmic domain of CTLA-4 using a novel protein transduction domain prevents allergic inflammation. Nat Med 12 (5):574-579. doi:10.1038/nm1385
78. Hsieh-Li H M, Chang J G, Jong Y J et al. (2000) A mouse model for spinal muscular atrophy.

Nat Genet 24 (1):66-70. doi:10.1038/71709
79. Gogliotti R G, Hammond S M, Lutz C et al. (2010) Molecular and phenotypic reassessment of an infrequently used mouse model for spinal muscular atrophy. Biochem Biophys Res Commun 391 (1):517-522. doi:10.1016/j.bbrc.2009.11.090
80. Nizzardo M, Simone C, Salani S et al. (2014) Effect of combined systemic and local morpholino treatment on the spinal muscular atrophy Delta7 mouse model phenotype. Clin Ther 36 (3):340-356 e345. doi:10.1016/j.clinthera.2014.02.004
81. Crawford T O, Pardo C A (1996) The neurobiology of childhood spinal muscular atrophy.

Neurobiol Dis 3 (2):97-110. doi:10.1006/nbdi,1996.0010
82. Sumner C J (2006) Therapeutics development for spinal muscular atrophy. NeuroRx 3 (2):235-245. doi:10.1016/j.nurx.2006.01.010
83. Burki U, Keane J, Blain A et al. (2015) Development and Application of an Ultrasensitive Hybridization-Based ELISA Method for the Determination of Peptide-Conjugated Phosphorodiamidate Morpholino Oligonucleotides. Nucleic Acid Ther 25 (5):275-284. doi:10.1089/nat.2014.0528
84. Miyatake S, Mizobe Y, Tsoumpra M K et al. (2019) Scavenger Receptor Class A1 Mediates Uptake of Morpholino Antisense Oligonucleotide into Dystrophic Skeletal Muscle. Mol Ther Nucleic Acids 14:520-535. doi:10.1016/j.omtn.2019.01.008
85. Gait M J, Arzumanov A A, McClorey G et al. (2019) Cell-Penetrating Peptide Conjugates of Steric Blocking Oligonucleotides as Therapeutics for Neuromuscular Diseases from a Historical Perspective to Current Prospects of Treatment. Nucleic Acid Ther 29 (1):1-12. doi:10.1089/nat.2018.0747
86. Kariya S, Park G H, Maeno-Hikichi Y et al. (2008) Reduced SMN protein impairs maturation of the neuromuscular junctions in mouse models of spinal muscular atrophy. Hum Mol Genet 17 (16):2552-2569. doi:10.1093/hmg/ddn156
87. Rochette C F, Gilbert N, Simard L R (2001) SMN gene duplication and the emergence of the SMN2 gene occurred in distinct hominids: SMN2 is unique to Homo sapiens. Hum Genet 108 (3):255-266
88. Zou L L, Ma J L, Wang T et al. (2013) Cell-penetrating Peptide-mediated therapeutic molecule delivery into the central nervous system. Curr Neuropharmacol 11 (2):197-208. doi:10.2174/1570159X11311020006
89. Tsoumpra M K, Fukumoto S, Matsumoto T et al. (2019) Peptide-conjugate antisense based splice-correction for Duchenne muscular dystrophy and other neuromuscular diseases. EBioMedicine 45:630-645. doi:10.1016/j.ebiom.2019.06.036
90. Meloni B P, Mastaglia F L, Knuckey N W (2020) Cationic Arginine-Rich Peptides (CARPs): A Novel Class of Neuroprotective Agents With a Multimodal Mechanism of Action. Front Neurol 11:108. doi:10.3389/fneur.2020.00108
91. Hua Y, Sahashi K, Rigo F et al. (2011) Peripheral SMN restoration is essential for long-term rescue of a severe spinal muscular atrophy mouse model. Nature 478 (7367):123-126. doi:10.1038/nature10485
92. Michaud M, Arnoux T, Bielli S et al. (2010) Neuromuscular defects and breathing disorders in a new mouse model of spinal muscular atrophy. Neurobiol Dis 38 (1):125-135. doi:10.1016/j.nbd.2010.01.006
93. Hensel N, Kubinski S, Claus P (2020) The Need for SMN-Independent Treatments of Spinal Muscular Atrophy (SMA) to Complement SMN-Enhancing Drugs. Front Neurol 11:45. doi:10.3389/fneur.2020.00045
94. Boido M, Vercelli A (2016) Neuromuscular Junctions as Key Contributors and Therapeutic Targets in Spinal Muscular Atrophy. Front Neuroanat 10:6. doi:10.3389/fnana.2016.00006
95. Zhang Z, Pinto A M, Wan L et al. (2013) Dysregulation of synaptogenesis genes antecedes motor neuron pathology in spinal muscular atrophy. Proc Natl Acad Sci USA 110 (48):19348-19353. doi:10.1073/pnas.1319280110
96. Echigoya Y, Nakamura A, Nagata T et al. (2017) Effects of systemic multiexon skipping with peptide-conjugated morpholinos in the heart of a dog model of Duchenne muscular dystrophy, Proc Natl Acad Sci USA 114 (16):4213-4218. doi:10.1073/pnas.1613203114
97. Echigoya Y, Aoki Y, Miskew B et al. (2015) Long-term efficacy of systemic multiexon skipping targeting dystrophin exons 45-55 with a cocktail of vivo-morpholinos in mdx52 mice. Mol Ther Nucleic Acids 4:e225. doi:10.1038/mtna.2014.76
98. Passini M A, Bu J, Roskelley E M et al. (2010)CNS-targeted gene therapy improves survival and motor function in a mouse model of spinal muscular atrophy. J Clin Invest 120 (4):1253-1264. doi:10.1172/JC141615
99. Donadon I, Bussani E, Riccardi F et al. (2019) Rescue of spinal muscular atrophy mouse models with AAV9-Exon-specific U1 snRNA. Nucleic Acids Res 47 (14):7618-7632, doi:10.1093/nar/gkz469
100. Ren J, Ding X, Funk G D et al. (2009) Ampakine CX717 protects against fentanyl-induced respiratory depression and lethal apnea in rats. Anesthesiology 110 (6):1364-1370. doi:10.1097/ALN,Ob013e31819faa2a
101. Ren J, Ding X, Greer J J (2015) 5-HT1A receptor agonist Befiradol reduces fentanyl-induced respiratory depression, analgesia, and sedation in rats. Anesthesiology 122 (2):424-434. doi:10.1097/ALN.0000000000000490

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. (canceled)

2. (canceled)

3. A conjugate comprising an antisense oligonucleotide capable of inducing exon skipping in human dystrophin covalently attached to a cell penetrating peptide (CPP) comprising the amino acid sequence:
YArVRRrGPRGYArVRRrGPRr;
uppercase: L-amino acids, lowercase: D-amino acids.

4. The conjugate of claim 3, wherein the antisense oligonucleotide binds to a target in exon 44, exon 45, exon 46, exon 47, exon 48, exon 49, exon 50, exon 51, exon 52, exon 53, exon 54 and/or exon 55 of human dystrophin pre-mRNA.

5. The conjugate of claim 3, wherein such antisense oligonucleotide comprises or consists of any one of the following oligonucleotides:
5′-AAAACGCCGCCATTTCTCAACAGATCTGTC-3′,
5′-GACAACAGTTTGCCGCTGCCCAATGCCATC-3′,
5′-AGTTGCTGCTCTTTTCCAGGTTCAAGTGGG-3′,
5′-GTTTGAGAATTCCCTGGCGCAGGGGCAACT-3′,
5′-CAATTTCTCCTTGTTTCTCAGGTAAAGCTC-3′,
5′-CAGATGATTTAACTGCTCTTCAAGGTCTTC-3′,
5′-ATCTCTTCCACATCCGGTTGTTTAGCTTGA-3′,
5′-GTAAACGGTTTACCGCCTTCCACTCAGAGC-3′,
5′-GTGTCACCAGAGTAACAGTCTGAGTAGGAG-3′,
5′-GGTAATGAGTTCTTCCAACTGGGGACGCCT-3′,
5′-CCTCCGGTTCTGAAGGTGTTCTTGTACTTC-3′,
5′-GAGAAGTTTCAGGGCCAAGTCATTTGCCAC-3′,
and
5′-TCTTCCAAAGCAGCCTCTCGCTCACTCACC-3′.

6. A composition comprising a first conjugate comprising a first antisense oligonucleotide capable of inducing exon skipping in human dystrophin covalently attached to a cell penetrating peptide (CPP) comprising the amino acid sequence:
YArVRRrGPRGYArVRRrGPRr;
uppercase: L-amino acids, lowercase: D-amino acids and a second conjugate comprising a second antisense oligonucleotide capable of inducing exon skipping in human dystrophin covalently attached to the cell penetrating peptide (CPP); and
at least one other conjugate comprising another antisense oligonucleotide capable of inducing exon skipping in human dystrophin covalently attached to the cell penetratin peptide (CPP).

7. (canceled)

8. The composition of claim 6, comprising a peptide-conjugated antisense oligonucleotide targeting exon 45, a peptide-conjugated antisense oligonucleotide targeting exon 46, a peptide-conjugated antisense oligonucleotide targeting exon 47, a peptide-conjugated antisense oligonucleotide targeting exon 48, a peptide-conjugated antisense oligonucleotide targeting exon 49, a peptide-conjugated antisense oligonucleotide targeting exon 50, a peptide-conjugated antisense oligonucleotide targeting exon 51, a peptide-conjugated antisense oligonucleotide targeting exon 52, a peptide-conjugated antisense oligonucleotide targeting exon 53, a peptide-conjugated antisense oligonucleotide targeting exon 54 and a peptide-conjugated antisense oligonucleotide targeting exon 55.

9. The composition of claim 6, comprising a peptide-conjugated antisense oligonucleotide targeting exon 45, a peptide-conjugated antisense oligonucleotide targeting exon 47, a peptide-conjugated antisense oligonucleotide targeting exon 49, a peptide-conjugated antisense oligonucleotide targeting exon 51, a peptide-conjugated antisense oligonucleotide targeting exon 53 and a peptide-conjugated antisense oligonucleotide targeting exon 55.

10. The composition of claim 6, comprising a peptide-conjugated antisense oligonucleotide targeting exon 45, a peptide-conjugated antisense oligonucleotide targeting exon 47 and a peptide-conjugated antisense oligonucleotide targeting exon 53.

11. The composition of claim 6, comprising:
the peptide-conjugated antisense oligonucleotide having the sequence
5′-GACAACAGTTTGCCGCTGCCCAATGCCATC-3′,
the peptide-conjugated antisense oligonucleotide having the sequence
5′-AGTTGCTGCTCTTTTCCAGGTTCAAGTGGG-3′,
the peptide-conjugated antisense oligonucleotide having the sequence
5′-GTTTGAGAATTCCCTGGCGCAGGGGCAACT-3′,
the peptide-conjugated antisense oligonucleotide having the sequence
5′-CAATTTCTCCTTGTTTCTCAGGTAAAGCTC-3′,
the peptide-conjugated antisense oligonucleotide having the sequence
5′-CAGATGATTTAACTGCTCTTCAAGGTCTTC-3′,
the peptide-conjugated antisense oligonucleotide having the sequence
5′-ATCTCTTCCACATCCGGTTGTTTAGCTTGA-3′,
the peptide-conjugated antisense oligonucleotide having the sequence
5′-GTAAACGGTTTACCGCCTTCCACTCAGAGC-3′,
the peptide-conjugated antisense oligonucleotide having the sequence
5′-GTGTCACCAGAGTAACAGTCTGAGTAGGAG-3′,
the peptide-conjugated antisense oligonucleotide having the sequence
5′-GGTAATGAGTTCTTCCAACTGGGGACGCCT-3′,
the peptide-conjugated antisense oligonucleotide having the sequence
5′-CCTCCGGTTCTGAAGGTGTTCTTGTACTTC-3′,
the peptide-conjugated antisense oligonucleotide having the sequence
5′-GAGAAGTTTCAGGGCCAAGTCATTTGCCAC-3′, and
the peptide-conjugated antisense oligonucleotide having the sequence
5′-TCTTCCAAAGCAGCCTCTCGCTCACTCACC-3′; wherein one or more thymines in the sequences are optionally replaced with uracil.

12. The composition of claim 6, comprising:
the peptide-conjugated antisense oligonucleotide having the sequence
5′-GACAACAGTTTGCCGCTGCCCAATGCCATC-3′,
the peptide-conjugated antisense oligonucleotide having the sequence
5′-GTTTGAGAATTCCCTGGCGCAGGGGCAACT-3′,
the peptide-conjugated antisense oligonucleotide having the sequence
5′-ATCTCTTCCACATCCGGTTGTTTAGCTTGA-3′,
the peptide-conjugated antisense oligonucleotide having the sequence
5′-GTGTCACCAGAGTAACAGTCTGAGTAGGAG-3′,
the peptide-conjugated antisense oligonucleotide having the sequence
5′-CCTCCGGTTCTGAAGGTGTTCTTGTACTTC-3′, and
the peptide-conjugated antisense oligonucleotide having the sequence
5′-TCTTCCAAAGCAGCCTCTCGCTCACTCACC-3′; wherein one or more thymines in the sequences are optionally replaced with uracil.

13. The composition of claim 6, comprising:
the peptide-conjugated antisense oligonucleotide having the sequence
5′-GACAACAGTTTGCCGCTGCCCAATGCCATC-3′,
the peptide-conjugated antisense oligonucleotide having the sequence
5′-GTTTGAGAATTCCCTGGCGCAGGGGCAACT-3′,
the peptide-conjugated antisense oligonucleotide having the sequence
5′-ATCTCTTCCACATCCGGTTGTTTAGCTTGA-3′,
the peptide-conjugated antisense oligonucleotide having the sequence
5′-CCTCCGGTTCTGAAGGTGTTCTTGTACTTC-3′, and
the peptide-conjugated antisense oligonucleotide having the sequence
5′-TCTTCCAAAGCAGCCTCTCGCTCACTCACC-3′; wherein one or more thymines in the sequences are optionally replaced with uracil.

14. The composition of claim 6, comprising:
the peptide-conjugated antisense oligonucleotide having the sequence
5′-GACAACAGTTTGCCGCTGCCCAATGCCATC-3′,
the peptide-conjugated antisense oligonucleotide having the sequence
5′-ATCTCTTCCACATCCGGTTGTTTAGCTTGA-3′,
the peptide-conjugated antisense oligonucleotide having the sequence
5′-GTAAACGGTTTACCGCCTTCCACTCAGAGC-3′,
the peptide-conjugated antisense oligonucleotide having the sequence
5′-GGTAATGAGTTCTTCCAACTGGGGACGCCT-3′,
the peptide-conjugated antisense oligonucleotide having the sequence
5′-CCTCCGGTTCTGAAGGTGTTCTTGTACTTC-3′, and
the peptide-conjugated antisense oligonucleotide having the sequence
5′-TCTTCCAAAGCAGCCTCTCGCTCACTCACC-3′; wherein one or more thymines in the sequences are optionally replaced with uracil.

15. The composition of claim 6, comprising:
the peptide-conjugated antisense oligonucleotide having the sequence
5′-GACAACAGTTTGCCGCTGCCCAATGCCATC-3′,
the peptide-conjugated antisense oligonucleotide having the sequence
5′-GTAAACGGTTTACCGCCTTCCACTCAGAGC-3′, and
the peptide-conjugated antisense oligonucleotide having the sequence
5′-TCTTCCAAAGCAGCCTCTCGCTCACTCACC-3′; wherein one or more thymines in the sequences are optionally replaced with uracil.

16. (canceled)

17. A method of treating a subject having DMD, comprising: administering a therapeutically effective amount the peptide conjugate of claim 1.

18. The method of claim 17, wherein the subject is a human.

19. A conjugate comprising an antisense oligonucleotide capable of inducing exon inclusion in human SMN2 gene covalently attached to a cell penetrating peptide (CPP) comprising the amino acid sequence:
YArVRRrGPRGYArVRRrGPRr;
uppercase: L-amino acids, lowercase: D-amino acids.

20. The conjugate of claim 19, wherein the antisense binds to intronic splicing silencer N1 of SMN2 pre-mRNA.

21. The conjugate of claim 19, wherein the antisense oligonucleotide comprises or consists of the sequence 5′-TCACTTTCATAATGCTGG-3′ and wherein the thymines are optionally replaced with uracil.

22. The conjugate of claim 19, wherein the antisense oligonucleotide is a phosphorodiamidate morpholino oligomer.

23. A method of treating spinal muscular atrophy (SMA) in a subject, comprising: administering a therapeutically effective amount of the conjugate of claim 19.
